SEQUENCE LISTING (12 sequences)

SEQ ID NO: 1 - DNA, 20 bp, Artificial Sequence
  Synthetic construct
ggttatgctttacatccaga

SEQ ID NO: 2 - DNA, 34 bp, Artificial Sequence
  Synthetic construct
gacatggtatgctttacatcagatggaaaactcc

SEQ ID NO: 3 - DNA, 35 bp, Artificial Sequence
  Synthetic construct
gacatggtatgctttacatccagatggaaaactcc

SEQ ID NO: 4 - DNA, 24 bp, Artificial Sequence
  Synthetic construct
gtttgacatggttatgcttactcc

SEQ ID NO: 5 - DNA, 20 bp, Artificial Sequence
  Synthetic construct
gacatggttatgcttactcc

SEQ ID NO: 6 - DNA, 36 bp, Artificial Sequence
  Synthetic construct
gacatggttatgctttacatccagatggaaaactcc

SEQ ID NO: 7 - DNA, 48 bp, Artificial Sequence
  Synthetic construct
caaaagatcctgcccactggaggatgtaaagcataaccatgtcaaact

SEQ ID NO: 8 - DNA, 49 bp, Artificial Sequence
  Synthetic construct
caaaagatcctgccccactggaggatgtaaagcataaccatgtcaaact

SEQ ID NO: 9 - DNA, 60 bp, Artificial Sequence
  Synthetic construct
caaaagatcctgccccactggagttttccatctggatgtaaagcataaccatgtcaaact

SEQ ID NO: 10 - DNA, 37 bp, Artificial Sequence
  Synthetic construct
caaaagatccggatgtaaagcataaccatgtcaaact

SEQ ID NO: 11 - DNA, 37 bp, Artificial Sequence; identical to SEQ ID NO: 10
  Synthetic construct

SEQ ID NO: 12 - DNA, 61 bp, Artificial Sequence
  Synthetic construct
caaaagatcctgccccactggagttttccatctggatgtaaagcataaccatgtcaaaact

(19) United States
(12) Patent Application Publication
(10) Pub. No.: US 2024/0209314 A1
(43) Pub. Date: Jun. 27, 2024

(54) T CELLS COMPRISING AN UNREARRANGED T CELL RECEPTOR (TCR) GENE LOCUS AND METHODS OF USE THEREOF

(71) Applicant: Sunnybrook Research Institute, Toronto (CA)

(72) Inventor: Juan Carlos Zuniga-Pflucker, Toronto (CA)

(21) Appl. No.: 18/287,970

(22) PCT Filed: Apr. 22, 2022
(86) PCT No.: PCT/CA2022/050622

§ 371 Date: Oct. 23, 2023

Related U.S. Application Data
(60) Provisional application No. 63/178,990, filed on Apr. 23, 2021.

(51) Int. Cl. C12N 5/0783; A61K 39/00; C07K 14/725

(52) U.S. Cl. CPC C12N 5/0636; A61K 39/4611; A61K 39/4632; C07K 14/7051; C12N 2501/125; C12N 2501/2307; C12N 2501/26; C12N 2506/45; C12N 2510/00

(57) ABSTRACT

A method of generating stem cells that are unable to undergo T cell receptor (TCR) or B cell receptor (BCR) gene rearrangements is provided. In particular, methods, compositions and kits for use in generating cells of the T cell lineage or B cell lineage comprising an unrearranged TCR gene locus or BCR gene locus, respectively, are 5 provided. In one embodiment, the cells are further engineered to express a TCR, bCR or CAR conferring specificity to an antigen of interest. Cells, compositions, kits and uses thereof are also provided.

### CROSS-REFERENCE TO RELATED APPLICATION

This application claims the benefit of priority to U.S. Provisional Application No. 63/178,990 filed Apr. 23, 2021, the contents of which are incorporated herein by reference in their entirety.

### FIELD

The application relates to stem cells that are unable to undergo T cell receptor (TCR) or B cell receptor (BCR) gene rearrangements. In particular, the application relates to methods, compositions and kits for use in generating cells of the T cell lineage or B cell lineage comprising an unrearranged TCR gene locus or BCR gene locus, respectively.

### BACKGROUND

The ability of T cells to specifically recognize antigens is accomplished through the expression of specific T cell receptors encoded by the uniquely rearranged genomic loci of the TCRα and β chains. T cell development is predicated on the successful rearrangement of the T cell receptor (TCR) gene loci, which encode for antigen-specific receptors.

The V(D)J Recombination Activating Genes (RAG) 1 and 2 are two essential DNA processing enzymes required for the rearrangement of the B cell receptor and T cell receptor gene loci (Schatz at al., 1989; Oettinger et al, 1990). RAG1/2 initiate V(D)J recombination by forming a complex that first recognizes and binds to recombination signal sequences (RSS) found adjacent to each V, D, and J gene segment. Upon formation of a synapse with another RSS, the RAG complex induces a double-strand DNA break, which is repaired by non-homologous end-joining process (Jones and Gellert, 2004; Smith et al, 2019). This ultimately results in the imperfect joining of different V, D, and J gene segments to potentially generate millions of different antigen receptors from a few hundred V(D)J segments.

### SUMMARY

The inventors differentiated human pluripotent stem cells with a CRISPR/Cas9-directed deletion of the RAG2 gene (RAG2-KO) and showed that human RAG2-deficient developing T cells progress up to the CD4+CD8+ double positive stage. The inventors also showed that expression of a rearranged TCRβ chain promotes cell survival and/or proliferation of developing human T-cells at the double positive stage.

Accordingly, the disclosure provides a method of generating stem or progenitor cells unable to undergo T cell receptor (TCR) gene rearrangements (TCR), the method comprising:
(a) culturing a sample comprising stem cells or progenitor cells,
wherein expression of at least one gene or protein required for V(D)J recombination in the stem cells or progenitor cells is reduced or eliminated compared to wildtype stem cells or progenitor cells.

In one embodiment, the method further comprises (b) isolating cells of the T cell lineage.

In another embodiment, the at least one gene or protein required for V(D)J recombination is RAG1 and/or RAG2.

In another embodiment, the at least one gene or protein required for V(D)J recombination is selected from the group consisting of Artemis, DNA-dependent protein kinase (DNA-PK), X-ray repair cross-complementing protein 4 (XRCC4), DNA ligase IV, non-homologous end-joining factor 1 (NHEJ1), Paralog of XRCC4 and XLF (PAXX), DNA polymerase λ and DNA polymerase μ.

In another embodiment, the stem cells are pluripotent stem cells. Optionally, the pluripotent stem cells are embryonic stem cells or induced pluripotent stem cells (iPSCs).

In another embodiment, the stem cells or progenitor cells are human cells.

In another embodiment, the cells of the T cell lineage are progenitor T (proT) cells, optionally CD45+CD34+CD7+ progenitor T (proT) cells.

In another embodiment, the cells of the T cell lineage are CD4+CD8+ double positive cells or CD4+CD8+CD3+ double positive cells.

In another embodiment, the cells of the T cell lineage are CD8+CD3+ single positive cells or CD4+CD3+ single positive cells.

In another embodiment, the method further comprises engineering the stem cells or progenitor cells or the cells of the T cell lineage to comprise at least one of a nucleic acid encoding a T cell receptor (TCR), a TCRβ chain and a chimeric antigen receptor (CAR).

In another embodiment, the stem cells or progenitor cells or the cells of the T cell lineage express the at least one of a T cell receptor (TCR), a TCRβ chain and a chimeric antigen receptor (CAR).

In another embodiment, the method further comprises engineering the stem cells or progenitor cells or the cells of the T cell lineage to comprise a nucleic acid encoding a TCRβ chain. In a further embodiment, the stem cells or progenitor cells or the cells of the T cell lineage that comprise a nucleic acid encoding a TCRβ chain do not comprise a nucleic acid that encodes a TCRβ chain or a chimeric antigen receptor (CAR).

In another embodiment, the stem cells or progenitor cells or the cells of the T cell lineage express a TCRβ chain.

In another embodiment, the stem cells or progenitor cells or the cells of the T cell lineage comprise a nucleic acid encoding a TCRβ chain and a nucleic acid encoding a CAR.

In another embodiment, the TCR or CAR confers specificity to an antigen, optionally a tumor-associated antigen, viral antigen or self antigen.

The disclosure also provides a cell of the T cell lineage, wherein the cell is generated by a method described herein.

In one embodiment, the cell of the T cell lineage is a CD4+CD8+ double positive cell or a CD4+CD8+CD3+ double positive cell.

In another embodiment, the cell is a CD45+CD34+CD7+ progenitor T cell, CD8+CD3+ single positive cell or CD4+CD3+ single positive cell.

The disclosure also provides a stem or progenitor cell, wherein expression of at least one gene or protein required for V(D)J recombination in the stem cell or progenitor cell is reduced or eliminated compared to a wild-type stem cell or progenitor cell.

In one embodiment, the at least one gene or protein required for V(D)J recombination is RAG1 and/or RAG2.

In another embodiment, the at least one gene or protein required for V(D)J recombination is selected from the group consisting of Artemis, DNA-dependent protein kinase (DNA-PK), X-ray repair cross-complementing protein 4 (XRCC4), DNA ligase IV, non-homologous end-joining factor 1 (NHEJ1), Paralog of XRCC4 and XLF (PAXX), DNA polymerase λ and DNA polymerase μ.

In another embodiment, the stem or progenitor cell further comprises at least one of a nucleic acid encoding a T cell receptor (TCR), a TCRβ chain and a chimeric antigen receptor (CAR).

In another embodiment, the stem or progenitor cell further comprises a nucleic acid encoding a TCRβ chain. In a further embodiment, the stem or progenitor cell that comprises a nucleic acid encoding a TCRβ chain does not comprise a nucleic acid that encodes a TCRβ chain or a chimeric antigen receptor (CAR).

In another embodiment, the stem cell is a pluripotent stem cell, optionally an embryonic stem cell or induced pluripotent stem cell (iPSC).

In another embodiment, the stem cell or progenitor cell is a human cell.

The disclosure also provides a use of a stem or progenitor cell as described herein for generating cells of the T cell lineage.

The disclosure also provides a kit comprising (i) a stem or progenitor cell as described herein and (ii) instructions for use of the stem or progenitor cell as described herein for generating cells of the T cell lineage.

The disclosure further provides a method of treating a disease or condition in a subject comprising:
(i) culturing a sample comprising stem cells or progenitor cells, wherein expression of at least one gene or protein required for V(D)J recombination in the stem cells or progenitor cells is reduced or eliminated compared to wildtype stem cells or progenitor cells, and
(ii) administering an effective amount of the cells or progenitor cells to a subject in need thereof,
wherein the stem cells or progenitor cells are engineered to comprise at least one of a nucleic acid encoding a T cell receptor (TCR) and a chimeric antigen receptor (CAR) that confers specificity to an antigen.

The disclosure also provides a method of treating a disease or condition in a subject comprising:
(i) culturing a sample comprising stem cells or progenitor cells, and isolating cells of the T cell lineage wherein expression of at least one gene or protein required for V(D)J recombination in the stem cells or progenitor cells is reduced or eliminated compared to wildtype stem cells or progenitor cells, and
(ii) administering an effective amount of the cells of the T cell lineage to a subject in need thereof,
wherein the stem cells or progenitor cells or the cells of the T cell lineage are engineered to comprise at least one of a nucleic acid encoding a T cell receptor (TCR) and a chimeric antigen receptor (CAR) that confers specificity to an antigen.

In one embodiment, the at least one gene or protein required for V(D)J recombination is RAG1 and/or RAG2.

In another embodiment, the at least one gene or protein required for V(D)J recombination is selected from the group consisting of Artemis, DNA-dependent protein kinase (DNA-PK), X-ray repair cross-complementing protein 4 (XRCC4), DNA ligase IV, non-homologous end-joining factor 1 (NHEJ1), Paralog of XRCC4 and XLF (PAXX), DNA polymerase λ and DNA polymerase μ.

In another embodiment, the disease is cancer and the antigen is a tumor-associated antigen.

The disclosure also provides a method of generating stem or progenitor cells unable to undergo T cell receptor (BCR) gene rearrangements, the method comprising:
(a) culturing a sample comprising stem cells or progenitor cells,
wherein expression of at least one gene or protein required for V(D)J recombination in the stem cells or progenitor cells is reduced or eliminated compared to wildtype stem cells or progenitor cells.

In one embodiment, the method further comprises (b) isolating cells of the B cell lineage.

In another embodiment, the at least one gene or protein required for V(D)J recombination is RAG1 and/or RAG2.

In another embodiment, the at least one gene or protein required for V(D)J recombination is selected from the group consisting of Artemis, DNA-dependent protein kinase (DNA-PK), X-ray repair cross-complementing protein 4 (XRCC4), DNA ligase IV, non-homologous end-joining factor 1 (NHEJ1), Paralog of XRCC4 and XLF (PAXX), DNA polymerase λ and DNA polymerase μ.

In another embodiment, the stem cells are pluripotent stem cells, optionally embryonic stem cells or induced pluripotent stem cells (iPSCs).

In another embodiment, the stem cells or progenitor cells are human cells.

In another embodiment, the cells of the B cell lineage are CD20+ or CD19+ cells.

In another embodiment, the cells of the B cell lineage are Tumor-Infiltrating B Cells (TIBs).

In another embodiment, the method further comprises engineering the stem cells or progenitor cells or the cells of the B cell lineage to comprise at least one of a nucleic acid encoding a B cell receptor (BCR), a BCRβ chain and a chimeric antigen receptor (CAR).

In another embodiment, the stem cells or progenitor cells or the cells of the B cell lineage express the B cell receptor (BCR), the BCRβ chain or the chimeric antigen receptor (CAR).

In another embodiment, the BCR or CAR confers specificity to an antigen, optionally a tumor-associated antigen, viral antigen or self antigen.

The disclosure also provides a cell of the B cell lineage, wherein the cell is generated by a method as described herein.

In one embodiment, the cell of the B cell lineage is a CD20+ or CD19+ cell.

In another embodiment, the cell of the B cell lineage is a Tumor-Infiltrating B Cell (TIB).

The disclosure also provides a use of a stem or progenitor cell as described herein for generating cells of the B cell lineage.

The disclosure also provides a kit comprising (i) a stem or progenitor cell as described herein and (ii) instructions for use of the stem or progenitor cell as described herein for generating cells of the B cell lineage.

The disclosure also provides a method of treating a disease or condition in a subject comprising:
(i) culturing a sample comprising stem cells or progenitor cells wherein expression of at least one gene or protein required for V(D)J recombination in the stem cells or progenitor cells is reduced or eliminated compared to wildtype stem cells or progenitor cells, and
(ii) administering an effective amount of the stem cells or progenitor cells to a subject in need thereof, wherein the stem cells or progenitor cells are engineered to comprise a nucleic acid encoding a B cell receptor (BCR) that confers specificity to an antigen.

The disclosure also provides a method of treating a disease or condition in a subject comprising:
(i) culturing a sample comprising stem cells or progenitor cells, and isolating cells of the B cell lineage wherein expression of at least one gene or protein required for V(D)J recombination in the stem cells or progenitor cells is reduced or eliminated compared to wildtype stem cells or progenitor cells, and
(ii) administering an effective amount of the cells of the B cell lineage to a subject in need thereof, wherein the stem cells or progenitor cells or the cells of the B cell lineage are engineered to comprise a nucleic acid encoding a B cell receptor (BCR) that confers specificity to an antigen.

In one embodiment, the at least one gene or protein required for V(D)J recombination is RAG1 and/or RAG2.

In another embodiment, the at least one gene or protein required for V(D)J recombination is selected from the group consisting of Artemis, DNA-dependent protein kinase (DNA-PK), X-ray repair cross-complementing protein 4 (XRCC4), DNA ligase IV, non-homologous end-joining factor 1 (NHEJ1), Paralog of XRCC4 and XLF (PAXX), DNA polymerase λ and DNA polymerase μ.

In another embodiment, the disease is cancer and the antigen is a tumor-associated antigen.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### BRIEF DESCRIPTION OF THE DRAWINGS

Embodiments of the disclosure will now be described in relation to the drawings in which:

FIG. 1 (A-E) shows generation and characterization of RAG2-KO hESC lines. FIG. 1A shows Sanger Sequencing of genomic DNA of CRISPR/Cas9-targeted RAG2 of clones 1 and 4. The altered allele is depicted in chromatograph and sequence above it, while alignment of the altered and WT alleles are shown below. FIG. 1B shows an immunoblot for RAG2 protein expression of in vitro-derived T-lineage cells from RAG2-KO clones (1 and 4) and Control WT hESCs, or Control PBMCs obtained from a T-ALL patient. GAPDH serves as a loading control. FIG. 1C shows immunofluorescence of RAG2-KO hESC clones for pluripotency markers. The scale bar corresponds to 100 μm.

FIG. 1D shows immunohistochemistry of RAG2-KO hESC clones against ectoderm (neural tissue), mesoderm (cartilage), and endoderm (glandular tissue) lineages of a teratoma formation assay. FIG. 1E shows CD34+ hemogenic endothelial cell yield and enrichment after 8 days of embryoid body differentiation from Control WT, RAG2-KO-1, and RAG2-KO-4 hESCs. (n=3 of three independent experiments).

FIG. 2 (A-B) shows T cell development from Control WT and RAG2-KO hESC lines. FIG. 2A shows a representative flow cytometry analysis of Control WT and RAG2-KO (clones 1 and 4) hPSC-derived T-lineage cells from d8 EBs+10-34d OP9-DL4-7FS co-cultures, as indicated. Cells were pre-gated for DAPI CD45+. (n=5 of five independent experiments). FIG. 2B shows a cell numbers of Control WT and RAG2-KO (clones 1 and 4) hPSC-derived T-lineage cells per 10,000 input of CD34+ cells after indicated number of days of culture on OP9-DL4-7FS. (n=3 of three independent experiments).

FIG. 3 (A-B) shows forced expression of a rearranged TCRβ chain in RAG2-KO CD4+CD8+DP cells results in cell expansion. FIG. 3A shows a schematic of the experimental approach. FIG. 3B shows cell counts of RAG2-KO DPs (clones 1 and 4) retrovirally-transduced with empty vector (dTomato), TCR alpha chain (TRA 1383i), or TCR beta chain (TRB 1383i) per 10,000 input of CD34+ cells. Transduced T-lineage cells (dTomato+) were first sorted for CD7+CD5+CD4+CD8+ double positive cells and then re-cultured on OP9-DL4-7FS for 10 days. (n=3 of three independent experiments).

FIG. 4 (A-B) shows TCR gene expression and CD4+CD8+ signature gene expression. FIG. 5A shows a heatmap analysis of specific TCR genes expressed in Control WT DPs but absent in RAG2-KO1/4 DPs and TCRβ-transduced RAG2-KO1/4 DPs. FIG. 5B shows expression of CD4⁺CD8⁺signature genes (as determined by thymus DP signature genes shown in Table 3) in RAG2-KO, TCRβ-transduced RAG2-KO, and umbilical cord blood (UCB) derived DPs. (n=2 for Control WT, RAG2-KO-1, RAG2-KO-4, and UCB, and n=1 for RAG2-KO-1 TCRβ-transduced and RAG2-KO-4 TCRβ-transduced of one independent experiment).

FIG. 5 (A-B) shows transcriptomic analysis of Control WT, RAG2-KO, and RAG2-KO TCRβ-transduced CD4⁺CD8⁺ DP cells. RNA-seq analysis of Control WT, RAG2-KO1/4, and TCRβ+ RAG2-KO1/4 CD4⁺CD8⁺DPs. Shown are genes that are differentially highly expressed in Control WT DPs compared to RAG2-KO1/4 DPs (FIG. 5A) and differentially highly expressed in RAG2-KO1/4 DPs compared to Control WT DPs (FIG. 5B). (n=2 for Control WT, RAG2-KO-1, and RAG2-KO-4, and n=1 for RAG2-KO-1 TCRβ-transduced and RAG2-KO-4 TCRβ-transduced of one independent experiment).

FIG. 6 (A-B) shows an analysis of proliferation and differentiation genes and biological pathways. FIG. 6A is a heatmap analysis of differentially expressed genes in DPs from RAG2-KO1/4 (KO) and TCRβ-transduced RAG2-KO1/4 (KOTCRb) cells. FIG. 6B shows names of gene ontology biological pathways that involve genes that are differentially up-regulated in Control WT DPs compared to RAG2-KO DPs. Highlighted are biological pathways that are relevant to cell survival and/or proliferation. Within the group of biological pathways that are involved in specific aspects of leukocyte regulation, the specific genes involved are also indicated.

FIG. 7 (A-B) shows forced expression of TCRαβ and TCRγδ chains in RAG2-KO CD4+CD8+ DPs. FIG. 7A shows TCRαβ- and TCRγδ-transduced DPs displayed higher cell numbers after 4 and 10 days of culture compared to dTomato-transduced DPs. From left to right are dTomato, TCRαβ- and TCRγδ. FIG. 7B is a flow cytometric analysis of TCR-transduced RAG2-KO DP cells showing expression of the corresponding αβ and γδ TCRs, respectively, on the cell surface of T-lineage cells.

### DETAILED DESCRIPTION

As described above, the inventors differentiated human pluripotent stem cells with a CRISPR/Cas9-directed deletion of the RAG2 gene (RAG2-KO) and showed that human RAG2-deficient developing T cells progress up to the CD4+CD8+ stage. The inventors also showed that RAG2-KO CD4+CD8+ double positive cells can be engineered to express TCRαβ and TCRγδ chains and that expression of a rearranged TCRβ chain promotes cell survival and/or proliferation of developing human T-cells at the double positive stage.

### I. Method for Generating Cells

Accordingly, the disclosure provides a method of generating stem or progenitor cells unable to undergo T cell receptor (TCR) gene rearrangements, the method comprising:
(a) culturing a sample comprising stem cells or progenitor cells,
   wherein expression of at least one gene or protein required for V(D)J recombination in the stem cells or progenitor cells is reduced or eliminated compared to wildtype stem cells or progenitor cells. In one embodiment, the method further comprises (b) isolating cells of the T cell lineage.

The disclosure also provides a method of generating a cell of the T cell lineage comprising an unrearranged T cell receptor (TCR) gene locus, the method comprising: (a) culturing a sample comprising stem cells or progenitor cells, wherein the expression of at least one gene or protein required for V(D)J recombination in the stem cells or progenitor cells is reduced or eliminated compared to wildtype stem or progenitor cells.

The term “cells of the T cell lineage” refers to cells that show at least one phenotypic characteristic of a T cell or a precursor or progenitor thereof that distinguishes the cells from other lymphoid cells, and cells of the erythroid or myeloid lineages. Such phenotypic characteristics can include expression of one or more proteins specific for T lineage commitment on cells or a precursor or progenitor thereof, or a physiological, morphological, functional, or immunological feature specific for a T cell.

In one embodiment, the cells of the T cell lineage are human cells.

As used therein, the term “a cell” or “the cell” includes a plurality of cells.

As used therein, the term “isolated” means that a cell has been separated or purified from cellular or biological material found with the cells in their native environment. It thus distinguishes the cells from how they exist in nature.

The disclosure also provides a method of generating stem or progenitor cells unable to undergo B cell receptor (BCR) gene rearrangements, the method comprising:
(a) culturing a sample comprising stem cells or progenitor cells,
wherein expression of at least one gene or protein required for V(D)J recombination in the stem cells or progenitor cells is reduced or eliminated compared to wildtype stem cells or progenitor cells. In one embodiment, the method further comprises (b) isolating cells of the B cell lineage.

The disclosure further provides a method of generating a cell of the B cell lineage comprising an unrearranged B cell receptor (BCR) gene locus, the method comprising: (a) culturing a sample comprising stem cells or progenitor cells, and (b) isolating cells of the B cell lineage, wherein the expression of at least one gene or protein required for V(D)J recombination in the stem cells or progenitor cells is reduced or eliminated compared to wildtype stem or progenitor cells.

The term “cells of the B cell lineage” refers to cells that show at least one phenotypic characteristic of a B cell or a precursor or progenitor thereof that distinguishes the cells from other lymphoid cells, and cells of the erythroid or myeloid lineages. Such phenotypic characteristics can include expression of one or more proteins specific for B-lineage on cells or a precursor or progenitor thereof, or a physiological, morphological, functional, or immunological feature specific for a B cell. In another embodiment, the cells of the B cell lineage are CD20+ or CD19+ cells. In another embodiment, the cells of the B cell lineage are Tumor-Infiltrating B Cells (TIBs).

In one embodiment, the cells of the B cell lineage are human cells.

Cells of the T cell lineage may be (a) progenitor or precursor cells committed to the T cell lineage (“progenitor T cells” or “proT cells”, as described herein); (b) CD7+ immature T cells; (c) cells that have undergone CD4 or CD8 lineage commitment (e.g. CD4+CD8^{lo}CR^{int} cells); (d) characterized by TCR gene rearrangement; (e) precursor thymocytes that are CD4+CD8+ double positive (DP); (f) CD4-CD8+ or CD4+CD8− and optionally TCR^{hi}; (g) CD3+CD90+; (h) single positive (SP) cells that are CD4-CD8+ or CD4+CD8− and TCR; (i) TCR-αβ+ and/or TCR-γδ⁺; (j) characterized by expression of any of multiple VB chains (e.g. VB-3, -6, and 17a); or (k) mature and functional or activated T cells which may be characterized as TCR/CD3^{hi}, CD4-CD8+ or CD4+CD8−

In one embodiment, a cell of the T cell lineage is a “progenitor T cell” or “proT cell”. The term “progenitor T cell” or “proT cell” as used herein means a T cell that is capable of maturing into a mature T cell or lymphocyte. In one embodiment, the pro T cell is a CD45+CD34+CD7+ proT cell.

In another embodiment, the progenitor T cell is a human progenitor T cell. Phenotypes of human progenitor T cells include CD34+CD7+ and CD7+CD5+CD1a−.

The present inventors showed that human RAG2-deficient developing T cells progress up to the CD4+CD8+ double positive stage. Accordingly, in another embodiment, a cell of the T cell lineage is a CD4 and CD8 double positive (DP) cell characterized by a CD4+CD8+ or CD4+CD8+CD3+ phenotype.

In another embodiment, a cell of the T cell lineage is a CD4 or CD8 single positive (SP) cell characterized by a CD4-CD8+, CD4+CD8- or CD4-CD8+CD3+, CD4+CD8-CD3+ phenotype.

As used herein an “unrearranged T-cell receptor (TCR) gene locus” refers to a TCR gene locus which has not undergone rearrangement and remains in the germline configuration. Similarly, the term “unrearranged B-cell receptor (BCR) gene locus” refers to a BCR gene locus which has not undergone rearrangement and remains in the germline configuration.

The TCR gene locus can comprise an alpha (a) chain and a beta (B) chain (encoded for example by genes TRA and TRB, respectively), or a gamma and delta (γ/δ) chains (encoded for example by genes TRG and TRD, respectively). As will be understood by a person of skill in the art, during development of a T cell, rearrangement of segments of the genes encoding the TCR gene locus occurs to encode for antigen-specific receptors.

As used herein, the term “gene or protein required for V(D)J recombination” refers to a gene of protein that is required for correct rearrangement of the B- or T-cell receptor (BCR or TCR) gene loci. In one embodiment, the gene or protein required for V(D)J recombination is a lymphocyte specific gene such as RAG1 or RAG2 or TdT.

In another embodiment, the gene or protein required for V(D)J recombination is a member of the non-homologous end joining (NHEJ) pathway for DNA repair.

In another embodiment, the gene or protein required for V(D)J recombination is Artemis, DNA-dependent protein kinase (DNA-PK), X-ray repair cross-complementing protein 4 (XRCC4), DNA ligase IV, non-homologous end-joining factor 1 (NHEJ1; also known as Cernunnos or XRCC4-like factor (XLF)), Paralog of XRCC4 and XLF (PAXX), or the DNA polymerases λ and μ.

As noted above, in one embodiment, the gene or protein required for V(D)J recombination is RAG1 or RAG2. The V(D)J Recombination Activating Genes (RAG) 1 and 2 encode two essential DNA processing enzymes required for the rearrangement of the B- and T-cell receptor (BCR or TCR) gene loci (Schatz at al, 1989; Oettinger et al, 1990). As described above, the inventors differentiated human pluripotent stem cells with a CRISPR/Cas9-directed deletion of the RAG2 gene (RAG2-KO) and showed that human RAG2-deficient developing T-cells progress up to the CD4+CD8+ stage.

Accordingly, in the methods disclosed herein, a cell of the T cell lineage or B cell lineage may be generated by culturing a sample comprising stem cells or progenitor cells, wherein the expression and/or function of RAG1 and/or RAG2 in the stem cells or progenitor cells is reduced or eliminated compared to wildtype stem cells or progenitor cells.

As used herein, RAG1 refers to V(D)J Recombination Activating Protein (RAG) 1, which is encoded by the RAG1 gene. The term “RAG1” includes RAG1 from any species or source. The term also includes sequences that have been modified from any of the known published sequences of RAG1 proteins and genes. RAG1 or the RAG1 gene may have any of the known published sequences for RAG1 which can be obtained from public sources such as GenBank. In one embodiment, RAG1 is human RAG1. Examples of human amino acid sequences for RAG1 include GenBank accession no. AAQ13571.1. Examples of human nucleic acid sequences for RAG1 include include GenBank no. NM_001377277.1 (Gene ID: 5896). The aforementioned sequences are incorporated herein by reference.

RAG2 refers to V(D)J Recombination Activating Protein (RAG) 2, which is encoded by the RAG2 gene. The term “RAG2” includes RAG2 from any species or source. The term also includes sequences that have been modified from any of the known published sequences of RAG2 proteins and RAG2 genes. RAG2 or the RAG2 gene may have any of the known published sequences for RAG2 which can be obtained from public sources such as GenBank. In one embodiment, RAG2 is human RAG2. Examples of the human amino acid sequences for RAG2 include GenBank accession no. AAH22397.1. Examples of human nucleic acid sequences for RAG2 include GenBank accession no. NM_000536.4 (Gene ID: 5897). The aforementioned sequences are incorporated herein by reference.

As used herein, the term “RAG1 expression” includes both RAG1 protein expression and RAG1 gene expression. Likewise, term “RAG2 expression” includes both RAG2 protein expression and RAG2 gene expression.

As used herein, the term “RAG1 function” and “RAG2 function” refers to the biological activity of the RAG1 and RAG2, respectively. For example, cells lacking the biological activity of RAG1 and/or RAG2 enzymes are unable undergo rearrangement of the B cell receptor and T cell receptor (BCR or TCR) gene loci.

Endogenous expression and/or function of a gene or protein required for V(D)J recombination (for example endogenous expression and/or function of RAG1 and/or RAG2) can be reduced or eliminated using any of a number of methods known in the art.

Reduced or eliminated expression and/or function of a gene or protein required for V(D)J recombination may be accomplished by mutation of deletion of the corresponding gene sequence. Reduced or eliminated expression and/or function of RAG1 or RAG2 may be accomplished for example by mutation or deletion of RAG1 or RAG2 gene sequences. For example, RAG1 or RAG2 gene sequences may be mutated or deleted from the genome of pluripotent stem cells using, for example, gene editing methods. Thus, for example, approaches employing RNA/DNA guided endonucleases (e.g., Clustered Regularly Interspersed Short Palindromic Repeats (CRISPR)/Cas9, Cpf1, and Argonaute), Transcription Activator-Like Effector (TALE)-nucleases, zinc finger nucleases (ZFN), or meganucleases can be adapted for use in embodiments of the present disclosure. In various examples, insertions or deletions are made by gene editing to cause a frame shift mutation, leading to gene knock out (i.e., lack of expression of a functional gene product).

Specific examples of protocols used in the present invention in the context of RAG2 include transfection of an expression vector containing cassettes for GFP, Cas9 endonuclease, a CRISPR chimeric cDNA and a gRNA moiety that targets RAG2.

In another embodiment, a RAG1 and/or RAG2 inhibitor is used to reduce endogenous RAG1 and/or RAG2 expression and/or function. The term “inhibitor” refers to an agent that reduces, decreases or otherwise blocks expression or activity of its target and includes any substance that is capable of inhibiting the expression or activity of the target and includes, without limitation, small molecules, antisense oligonucleotide molecules (antisense nucleic acid molecules), siRNAs or shRNAs, aptamers, proteins, antibodies (and fragments thereof), gene editing agents and other substances directed at the target expression or activity.

Endogenous RAG1 and/or RAG2 expression and/or function can be reduced transiently or permanently (for example, by introducing gene mutations or deletions into the germline).

As used herein, the term “wildtype” refers to a cell which has normal (non-modified), endogenous expression levels of a gene or protein required for V(D)J recombination. In one embodiment, the term “wildtype” refers to a cell which has normal (non-modified), endogenous expression levels of RAG1 and/or RAG2 genes or proteins. The wildtype cell is optionally a stem or progenitor cell.

In one embodiment of the present disclosure, endogenous RAG1 expression and/or function is reduced by at least 5%, 10%, 25%, 50%, 75% or 100% compared to a wild-type cell. In another embodiment, the cell has no detectable endogenous RAG1 expression and/or function.

In another embodiment, endogenous RAG2 expression and/or function is reduced by at least 5%, 10%, 25%, 50%, 75% or 100% compared to a wild-type cell. In another embodiment, the cell has no detectable endogenous RAG2 expression and/or function.

In one embodiment, the stem or progenitor cell is a pluripotent stem cell. As used herein, the term “pluripotent stem cell” refers to any stem cell having the potential to differentiate into all cell types of a human or animal body, not including extra-embryonic tissues. These stem cells include both embryonic stem cells (ESCs) and induced pluripotent cells (iPSCs). Hence, the cells suitable for the method of the present invention include stem cells selected from iPSCs and ESCs. In one embodiment, the pluripotent stem cells are human pluripotent stem cells (hPSCs) and they include human iPSCs (hiPSCs) and human ESCs (hESCs).

The term “embryonic stem cell” or “ESC” as used herein refers to undifferentiated embryonic stem cells that have the ability to integrate into and become part of the germ line of a developing embryo.

The term “induced pluripotent stem cell” or “iPSC” as used herein refers to cells derived from somatic cells, such as skin or blood cells that have been reprogrammed back into an embryonic-like pluripotent state. In one embodiment, iPSCs are derived from T cells with a known or unknown TCR specificity (for example, T cells bearing TCRs with specificity against cancer).

In one embodiment, the stem or progenitor cell is a hematopoietic stem or progenitor cell (HSPC). In another embodiment, the stem or progenitor cell is a CD34⁺ hematopoietic precursor cell, optionally a CD34⁺ hemogenic endothelial precursor cell that has been differentiated from an ESC or iPSC, or a CD34⁺ pre-hematopoietic cell differentiated from an ESC or pluripotent stem cell (PSC). Various differentiation protocols for obtaining CD34⁺ cells are known in the art. For therapeutic applications, the stem cells or progenitor cells used to generate the cells of the T cell lineage may be obtained from the patient to be treated.

Stem or progenitor cells may be obtained from any suitable source, including, without limitation, umbilical cord blood, embryos, embryonic tissue, fetal tissue, bone marrow and blood.

Typically, a sample containing stem or progenitor cells is first depleted of non-stem cells or mature cells. Negative and positive selection methods known in the art may be used for enrichment of the stem or progenitor cells. For example, cells can be sorted based on cell surface antigens using a fluorescence activated cell sorter, or magnetic beads which bind cells with certain cell surface antigens. Negative selection columns can be used to remove cells expressing lineage specific surface antigens.

In an embodiment, a sample containing stem or progenitor cells is separated into lineage-negative (Lin) and lineage position (Lint) fractions. The Lin fraction can be sorted for CD34⁺ cells.

The progenitor cells or stem cells may be cultured under suitable conditions to generate cells of the T cell lineage or B cell lineage. Methods of culturing progenitor cells or stem cells to generate cells of the T cell lineage or B cell lineage are known in the art.

For example, as described herein, human pluripotent stem cells may be induced to differentiate as embryoid bodies, then CD34+ cells may be isolated by magnetic-assisted cell-sorting and placed in co-culture with OP9-DL4 cells to induce their differentiation towards the T cell. Such a protocol is described in Kennedy et al. 2012.

In one embodiment, the cells are cultured in the presence of one or more Notch ligands conjugated to a suspension support for a sufficient time to form cells of the T cell lineage.

The progenitor cells or stem cells may be cultured on plates or in suspension in a bioreactor, optionally a closed or a closed, automated bioreactor. Various bioreactors are known in the art and can include batch, fed batch or continuous bioreactors. An example of a continuous bioreactor is a continuous stirred-tank reactor model.

Various concentrations of progenitor cells or stem cells in the culture are contemplated. For example, the concentration of progenitor cells or stem cells in the culture may be anywhere from 1 to millions of cells per ml of media.

One or more positive cytokines that promote commitment and differentiation of cells of the T cell lineage or B cell lineage may also be added to the culture. The cytokines may be human in origin, or may be derived from other species. The concentration of a cytokine in a culture is typically about 1-10 ng/ml. The following are representative examples of cytokines that may be employed in the present application to promote commitment and differentiation of cells of the T cell lineage: all members of the Flt-3-ligand, and interleukin-7 (IL-7) and Stem Cell Factor. In one embodiment, the cytokines used herein are Flt-3-ligand and IL-7 and Stem Cell Factor. The cytokines may be used in combination with equal molar or greater amounts of a glycosaminoglycan such as heparin sulfate. The cytokines are commercially available or can be produced by recombinant DNA techniques and purified to various degrees. Some of the cytokines may be purified from culture media of cell lines by standard biochemical techniques.

One or more additional molecules, optionally conjugated to a suspension support, may also be added to the culture. In one embodiment, the additional molecule is a molecule that promotes T cell development (for example, promotes commitment and differentiation of cells of T cell lineage), also referred to herein as a “T cell co-stimulatory molecule”. In one example, the inventors have shown that microbead-conjugated DL4 and VCAM1 cultured with HSPCs accelerated differentiation to the T cell lineage. Thus, in one embodiment, the T cell co-stimulatory molecule is VCAM1. As used herein, the term “VCAM1” refers to Vascular cell adhesion protein 1 also known as vascular cell adhesion molecule 1 (VCAM1) or cluster of differentiation 106 (CD106), a protein that in humans is encoded by the VCAM1 gene. The term “VCAM1” also includes a mutant or variant of a VCAM1. In another embodiment, the T cell co-stimulatory molecule is a cytokine or chemokine (Stem Cell Factor, IL-7, CCL25, or CXCR4), Major Histocompatibility Complex (MHC) class I or class II, or co-stimulatory (CD80, CD86) molecule. Optionally, the T cell co-stimulatory molecule comprises at least one protein tag. Various protein tags are known in the art and can be used for a number of purposes. In one embodiment, the T cell co-stimulatory molecule comprises an Fc tag (also known as an Fc-fusion protein).

The progenitor cells and stem cells may be cultured in culture media comprising conditioned media, non-conditioned media, or embryonic stem cell media. Examples of suitable conditioned media include IMDM, DMEM, or «MEM, conditioned with embryonic fibroblast cells (e.g. human embryonic fibroblast cells or mouse embryonic fibroblast cells), or equivalent media. Examples of suitable non-conditioned media include Iscove's Modified Dulbecco's Medium (IMDM), DMEM, or «MEM, or equivalent media. The culture media may comprise serum (e.g. bovine serum, fetal bovine serum, calf bovine serum, horse serum, human serum, or an artificial serum substitute) or it may be serum free.

In one embodiment, the culture conditions entail culturing the progenitor cells or stem cells for a sufficient period of time so that cells in the preparation form proT cells. In another embodiment, the culture conditions entail culturing the progenitor cells or stem cells for a sufficient period of time so that cells in the preparation form mature T cells, for example mature SP T cells. It will be appreciated that the cells may be maintained for the appropriate amount of time required to achieve the desired cellular composition. Optionally, the progenitor cells or stem cells are cultured for at least 6, 8, 10, 12, 14, 21, 28, 35 or 42 days.

In one embodiment, the method further comprises engineering the cells to comprise a nucleic acid encoding a T cell receptor (TCR) or a chimeric antigen receptor (CAR). In another embodiment, the method further comprises engineering the cells to comprise a nucleic acid encoding a T cell receptor beta chain (TCRβ).

The cells may be engineered to comprise a nucleic acid encoding a TCR, CAR and/or TCRβ at any time during the differentiation process. For example, in one embodiment, the stem or progenitor cells comprise a nucleic acid encoding a TCR, CAR and/or TCRβ. In another embodiment, the cells of the T cell lineage comprise a nucleic acid encoding a TCR, CAR and/or TCRβ. In further embodiments, the stem or progenitor cells and/or the cells of the T cell lineage express a TCR, CAR and/or TCRβ.

In one embodiment, the stem or progenitor cells or the cells of the T cell lineage comprise a nucleic acid encoding a TCRβ alone. In other words, the stem or progenitor cells or the cells of the T cell lineage comprise a nucleic acid encoding a TCRβ and do not comprise a nucleic acid encoding a TCR or CAR.

In another embodiment, the stem or progenitor cells or the cells of the T cell lineage comprise a nucleic acid encoding a TCRβ and a nucleic acid encoding a CAR. In such an embodiment, the TCRβ directs differentiation while the CAR can provide therapeutic efficacy.

In another embodiment, the method further comprises engineering the cells of the B-cell lineage to express a B cell receptor (BCR), B cell receptor beta chain (BCRβ) and/or CAR. The cells may be engineered to comprise a nucleic acid encoding a BCR, BCRβ and/or CAR at any time during the differentiation process. For example, in one embodiment, the stem or progenitor cells comprise a nucleic acid encoding a BCR, BCRβ and/or CAR. In another embodiment, the cells of the B cell lineage comprise a nucleic acid encoding a BCR, BCRβ and/or CAR. In further embodiments, the stem or progenitor cells and/or the cells of the B cell lineage express a BCR, BCRβ and/or CAR.

The cells may be engineered by any method known in the art to comprise a nucleic acid encoding a TCR, TCRβ, CAR, BCR or BCRβ. For example, the cells may be transformed with viral or non-viral vectors carrying a TCR, TCRβ, CAR, BCR or BCRβExamples of viral vectors include, but are not limited to, retroviruses (including lentivirus), adenoviruses and adeno-associated viruses. Examples of non-viral vectors include, but are not limited to, nude DNA, minicircle DNA vectors, liposomes, polymerizers and molecular conjugates.

The TCR, BCR or CAR optionally confers specificity to an antigen such as a tumour associated antigen, a viral antigen or a self-antigen.

A “tumor associated antigen” is an antigen produced by tumor cells. Examples of tumor associated antigens include, but are not limited to, alphafetoprotein (AFP), arcinoembryonic antigen (CEA), CA-125, MUC-1, Epithelial tumor antigen (ETA), tyrosinase, melanoma-associated antigen (MAGE), WT1 and NYESO1.

A “viral antigen” is an antigen encoded by a viral genome. Examples of viral antigens include, but are not limited to EBV, CMV, HIV and SARS viral antigens.

A “self-antigen” is an antigen that is produced by the subject. A self-antigen can be a tumor associated antigens. A T cell expressing a TCR or CAR conferring specificity to a self-antigen could be used for example to make regulatory T cells that block self-reactive T cells.

### II. Pluripotent Stem Cells

As described herein, the present inventors generated RAG2-deficient (RAG2-KO) human pluripotent stem cell (hPSC) lines.

Accordingly, the disclosure also provides an isolated stem or progenitor cell, wherein expression of at least one gene or protein required for V(D)J recombination, optionally RAG1 and/or RAG2, in the stem cell or progenitor cell is reduced or eliminated compared to a wildtype stem cell or progenitor cell. Optionally, the stem cell or progenitor cell is human cell.

In one embodiment, the stem cell is a pluripotent stem cell.

In another embodiment, the pluripotent stem cell is an embryonic stem cell or induced pluripotent stem cell (iPSC).

Reduced or eliminated expression and/or function of RAG1 or RAG2 may be accomplished by any one of a number of methods known in the art.

In one embodiment, the stem cell or progenitor cell comprises a mutated or deleted RAG1 and/or RAG2 gene sequence. For example, insertions or deletions in the RAG1 and/or RAG2 gene sequences may cause a frame shift mutation, leading to gene knock out (i.e., lack of expression of a functional gene product).

In one embodiment of the present disclosure, endogenous RAG1 expression and/or function is reduced in the stem cell or progenitor cell by at least 5%, 10%, 25%, 50%, 75% or 100% compared to a wild-type cell. In another embodiment, the cell has no detectable endogenous RAG1 expression and/or function.

In another embodiment, endogenous RAG2 expression and/or function is reduced in the stem cell or progenitor cell by at least 5%, 10%, 25%, 50%, 75% or 100% compared to a wild-type cell. In another embodiment, the cell has no detectable endogenous RAG2 expression and/or function.

In addition, in one embodiment, the stem cell or progenitor cell further comprises a nucleic acid encoding a TCR, CAR, TCRβ, BCR or BCRβ. In another embodiment, stem cell or progenitor cell expresses a TCR, CAR, TCRβ, BCR or BCRβ

In a further embodiment, the stem or progenitor cell comprises a nucleic acid encoding a TCRβ and does not comprise a nucleic acid encoding a TCR or CAR.

### IV. Cells of the T Cell or B Cell Lineage

The disclosure further provides cells of the T cell lineage or B cell lineage generated by the methods, systems and kits described herein, or mitotic or differentiated cells that are progeny of the cells.

In one embodiment, the disclosure provides a “progenitor T cell” or “proT cell” generated by the methods described herein. In another embodiment, the progenitor T cell is a human progenitor T cell, for example a human progenitor T cell characterized by CD34+CD7+, CD7+CD5+CD1a- or CD45+CD34+CD7+.

The disclosure also provides a double positive (DP) T-cell characterized by CD4+CD8+ or CD4+CD8+CD3+. The disclosure further provides a cell of the T-cell lineage that is a single positive (SP) cell characterized by CD4-CD8+, CD4+CD8- or CD8+CD3+, CD4+CD3+.

In one embodiment, the cells of the T cell lineage further comprise a nucleic acid encoding a TCR, CAR or TCRβ. In another embodiment, the cells of the T cell lineage express a TCR, CAR or TCRβ.

In a further embodiment, the cells of the T cell lineage comprise a nucleic acid encoding a TCRβ and does not comprise a nucleic acid encoding a TCR or CAR.

In another embodiment, a cell of the T cell lineage generated by the methods described herein (for example, a progenitor T cell or a mature T cell) is engineered with a T cell receptor (TCR) or a chimeric antigen receptor (CAR) that confers specificity to an antigen.

The antigen is optionally a tumor associated antigen (TAA), viral antigen or self-antigen. T-cells engineered to express a TCR or a CAR to confer specificity to a TAA can be useful for treating conditions such as cancer.

Likewise, in another embodiment, the cells of the B cell lineage further comprise a nucleic acid encoding a CAR, TCRβ, BCR or BCRβ. In another embodiment, the cells of the B cell lineage express a CAR, BCR or BCRβ

In another embodiment, a cell of the B cell lineage generated by the methods described herein is engineered with a B cell receptor (BCR) or a chimeric antigen receptor (CAR) that confers specificity to an antigen.

The antigen is optionally a tumor associated antigen (TAA), viral antigen or self-antigen. T-cells engineered to express a BCR or a CAR to confer specificity to a TAA can be useful for treating conditions such as cancer.

In another aspect, the present disclosure provides a pharmaceutical composition comprising isolated stem or progenitor cells or cells of the T cell lineage or

B cell lineage generated by the methods described herein and a pharmaceutically acceptable diluent or carrier.

Suitable diluents and carriers are described, for example, in Remington's Pharmaceutical Sciences. On this basis, the compositions include, albeit not exclusively, solutions of the proT cells in association with one or more pharmaceutically acceptable vehicles or diluents, and contained in buffered solutions with a suitable pH and iso-osmotic with physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween™), alcohols, polyols, glycerin and vegetable oils, for example. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to the patient.

Pharmaceutical compositions also include cyropreservative solutions. In one embodiment, cells of the T cell lineage generated by the methods described herein are cryopreserved in appropriate media, for example pharmaceutically acceptable or GMP-grade media and optionally formulated for administration to a subject in need thereof.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The compositions can be administered for example, by parenteral, intravenous, subcutaneous, intramuscular, intracranial, intraorbital, ophthalmic, intraventricular, intracapsular, intraspinal, intracisternal, intraperitoneal, intranasal, aerosol or oral administration. For parenteral administration, solutions of the pro-T cells described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations.

Preferably the cells of the T cell lineage or B cell lineage are present in an amount effective for treating a disease state in a subject need thereof. In one embodiment the cell of the T cell lineage is present in an amount effective to enhance hematopoietic progenitor cell engraftment in a subject in need thereof. Optionally, the composition further comprises cells of the T cell lineage, or tissue for transplantation. In one embodiment the tissue comprises a thymus. In another embodiment the tissue comprises an organ.

### III. Kits

Stem or progenitor cell as described herein may be prepared and packaged in kits for use in generating cells of the T cell lineage or cells of the B cell lineage.

Accordingly, also provided herein is a kit for producing cells of the T cell lineage or cells of the B cell lineage comprising a stem or progenitor cell, wherein the expression of RAG1 and/or RAG2 in the stem cell or progenitor cell is reduced or eliminated compared to wildtype stem cell or progenitor cells.

In one embodiment, the kit further comprises culture media for culturing a sample comprising stem cells or progenitor cells for producing cells of the T cell lineage or cells of the B cell lineage. Examples of culture media include conditioned media, non-conditioned media, or embryonic stem cell media. The culture media may comprise serum (e.g. bovine serum, fetal bovine serum, calf bovine serum, horse serum, human serum, or an artificial serum substitute) or it may be serum free.

In another embodiment, the kit further comprises one or more additional molecules. In one embodiment, the additional molecule is a molecule that promotes T cell development (for example, promotes commitment and differentiation of cells of T cell lineage), also referred to herein as a “T cell co-stimulatory molecule”. In another embodiment, the T cell co-stimulatory molecule is VCAM1.

The media optionally includes one or more cytokines that promote commitment and differentiation of cells of the T cell lineage or cells of the B cell lineage. The cytokines may be human in origin, or may be derived from other species. The concentration of a cytokine in a culture is typically about 1-10 ng/ml. The following are representative examples of cytokines that may be employed in the present application: all members of the Flt-3-ligand, and interleukin-7 (IL-7) and Stem Cell Factor. In one embodiment, the cytokines used herein are Flt-3-ligand and IL-7 and Stem Cell Factor. The cytokines may be used in combination with equal molar or greater amounts of a glycosaminoglycan such as heparin sulfate. The cytokines are commercially available or can be produced by recombinant DNA techniques and purified to various degrees. Some of the cytokines may be purified from culture media of cell lines by standard biochemical techniques.

In one embodiment, the kit comprises one or more containers for the within-described reagents.

Printed instructions providing guidance in the use of the reagent(s) may also be included in the kit, in various embodiments. The term “instructions” or “instructions for use” typically includes a tangible expression describing the cells, culturing time periods, temperature, media conditions, and the like. For example, in one embodiment, the instructions describe a method comprising culturing a sample comprising stem cells or progenitor cells.

### V. Therapeutic Applications

T cells and B cells engineered to recognize specific antigens have wide ranging therapeutic applications.

Accordingly, the disclosure provides method of treating a disease or condition in a subject comprising:
(i) culturing a sample comprising stem cells or progenitor cells, wherein expression of at least one gene or protein required for V(D)J recombination in the stem cells or progenitor cells is reduced or eliminated compared to wildtype stem cells or progenitor cells, and
(ii) administering an effective amount of the cells or progenitor cells to a subject in need thereof,
wherein the stem cells or progenitor cells are engineered to comprise at least one of a nucleotide sequence encoding a T cell receptor (TCR), a chimeric antigen receptor (CAR) or a B cell receptor (BCR) that confers specificity to an antigen.

The disclosure also provides a method of treating a disease or condition in a subject comprising:
(i) culturing a sample comprising stem cells or progenitor cells, wherein the expression of RAG1 and/or RAG2 in the stem cells or progenitor cells is reduced or eliminated compared to wildtype stem cells or progenitor cells, and
(ii) administering an effective amount of the cells of the T cell lineage to a subject in need thereof,
wherein the cells of the T cell lineage are engineered with a T cell receptor (TCR) or a chimeric antigen receptor (CAR) to confer specificity to an antigen.

The disclosure also provides a use of cells of the T cell lineage, generated by the methods described herein for treating a disease or condition in a subject, wherein the cells of the T cell lineage are engineered with a T cell receptor (TCR) or a chimeric antigen receptor (CAR) that confer specificity to an antigen. The disclosure further provides a use of cells of the T cell lineage, generated by the methods described herein for preparation of a medicament for treating a disease or condition in a subject, wherein the cells of the T cell lineage are engineered with a T cell receptor (TCR) or a chimeric antigen receptor (CAR) that confers specificity to an antigen.

The disclosure also provides cells of the T cell lineage, generated by the methods described herein for use in treating a disease or condition in a subject, wherein the cells of the T cell lineage are engineered with a T cell receptor (TCR) or a chimeric antigen receptor (CAR) that confers specificity to an antigen.

The disclosure also provides a method of treating a disease or condition in a subject comprising:
(i) culturing a sample comprising stem cells or progenitor cells, and isolating cells of the B cell lineage, wherein expression of at least one gene or protein required for V(D)J recombination in the stem cells or progenitor cells is reduced or eliminated compared to wildtype stem cells or progenitor cells, and
   (ii) administering an effective amount of the cells of the B cell lineage to a subject in need thereof,
   wherein the cells of the B cell lineage are engineered with at least one B cell receptor (TCR) to confer specificity to an antigen.

The disclosure also provides a use of cells of the B cell lineage, generated by the methods described herein for treating a disease or condition in a subject, wherein the cells of the B cell lineage are engineered with a B cell receptor (BCR) or CAR that confers specificity to an antigen. The disclosure further provides a use of cells of the B cell lineage, generated by the methods described herein for preparation of a medicament for treating a disease or condition in a subject, wherein the cells of the B cell lineage are engineered with a B cell receptor (BCR) or CAR that confers specificity to an antigen.

The disclosure also provides cells of the B cell lineage, generated by the methods described herein for use in treating a disease or condition in a subject, wherein the cells of the B cell lineage are engineered with a B cell receptor (BCR) or CAR that confers specificity to an antigen.

The antigen is optionally a tumor associated antigen (TAA), a viral antigen or a self-antigen. In one embodiment, the antigen is a TAA and the disease is cancer.

As used herein, the phrase “effective amount” or “therapeutically effective amount” means an amount effective, at dosages and for periods of time necessary to achieve the desired result. Effective amounts may vary according to factors such as the disease state, age, sex, weight of the subject. The amount of a given cell preparation that will correspond to such an amount will vary depending upon various factors. Such as the pharmaceutical formulation, the route of administration, the type of disease or disorder, the identity of the subject or host being treated, and the like, but can nevertheless be routinely determined by one skilled in the art. An “effective amount” will preferably be an amount effective for the cell of the T cell lineage to engraft the subject being treated.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also includes prophylactic treatment.

The term “subject” as used herein means any member of the animal kingdom and is preferably a human.

The following non-limiting examples are illustrative of the present application:

### EXAMPLES

### Example 1

### Materials and Methods:

hESC Maintenance. Human ESCs (H1; WiCell Research Institute, Madison, WI) were maintained and expanded on plates coated with Matrigel (Corning, NY, USA) in TeSR-E8 medium (STEMCELL Technologies, Vancouver, Canada). Cells were passaged by non-enzymatic dissociation using 0.5 mM EDTA.

Generation of RAG2-KO hESCs. The pD1321-GFP expression vector, containing cassettes for GFP, Cas9 endonuclease, a CRISPR chimeric cDNA, and the gRNA moiety designed to target RAG2 [GGTTATGCTTTACATCCAGA (SEQ ID NO: 1)], was custom synthesized (DNA2.0). After transfection with Lipofectamine 3000 (Life Technologies, Carlsbad, CA), GFP+ hESCs were sorted using flow cytometry. Individual clones were picked, expanded, and aliquots were collected for purification of genomic DNA using the Genomic DNA kit (Invitrogen). Mutations (indels) were validated by sequencing products of PCR amplification of the regions flanking the targeting sites. The RAG2 KO clone 1 exhibited a 1 bp deletion in one allele and 16 bp deletion in the other allele, while clone 4 exhibited a 11 bp deletion in one allele and 23 bp deletion in the other allele.

Western Blot Analysis. Briefly, cellular lysates were prepared by incubating the cells in lysis buffer (50 mM Tris-HCl, pH7.5, 150 mM NaCl, 0.5% NP-40, 2 mM EDTA) containing protease inhibitor cocktail (Roche) for 20 min at 4ºC, followed by centrifugation at 14000×g for 15 min at 4° C. Proteins were separated by SDS-PAGE, transferred onto PVDF membrane (Millipore, Louis, MO) and probed with anti-RAG2 antibody (Abcam—Ab95955; 1:1000 dilution) overnight at 4° C. followed by incubation with secondary antibody. The anti-RAG2 antibody used was a rabbit polyclonal made against a recombinant fragment corresponding to amino acids 271-519 of human RAG2 (Abcam), which are well beyond the gRNA targeting site. Immunoreactive bands were visualized using western blotting Luminol reagent (Thermo). PBMCs from a T-ALL patient were used as positive control (Bories et al, 1991).

Immunostaining. Cells were fixed in 4% paraformaldehyde, and permeabilization and blocking were performed in 5% NGS (Abcam, Cambridge, USA) and 1% Triton X-100 (Sigma-Aldrich, Louis, MO) in PBS for 30 min. Cells were stained and analyzed as previously described (Li et al, 2017).

Teratoma Formation. All animal studies were approved by Ethics Committee of Experimental Research of Peking University and were in accordance with the International Animal Care and Use Committee Guidelines. 6-8 week-old non-obese diabetic (NOD)/SCID mice were injected subcutaneously with 1×10⁷ hESCs resuspended in DMED-F12 with 50% Matrigel to allow teratoma formation for 8 weeks. Histological analysis was performed as previously described (Li et al, 2017).

hPSC Differentiation and CD34+ Isolation. hPSCs, H1 embryonic stem cells (ESC) were differentiated as previously described (Kennedy et al, 2012). After 8 days of differentiation, embryoid bodies were harvested and dissociated into single-cells using Collagenase type IV and trypsin-EDTA as previously described (Kennedy et al, 2012), and positively selected using a MACS column (Miltenyi Biotec) with anti-CD34 PE-conjugated antibody (BD Biosciences) and anti-PE microbeads. The cell yield and purity of the positive selection was assessed pre- and post-MACS by flow cytometry.

OP9-DL4-7FS Co-Culture and Differentiation. OP9-DL4 cells expressing hIL-7, hFLT3-L, and hSCF (7FS) were generated and grown in a-MEM containing 10-20% FBS (Gibco), 1% Pen-Strep (Life Technologies), and phospho-ascorbic acid (Sigma-Aldrich) at 37° ° C., 5% CO₂. For co-cultures, OP9-DL4-7FS cells were plated in 6-well plates the previous day. MACS-purified CD34+ cells were counted and seeded at a density of 5-20×10⁴ cells per 6-well plate, and differentiated by co-culture with OP9-DL4-7FS cells in OP9 medium at 37° C., 5% CO₂, and cell were passaged and co-cultured with fresh OP9-DL4-7FS cells every ˜5 days.

Retroviral Transduction. PG13 cell lines stably expressing empty vector dTomato, TCRα-dTomato, or TCRβ-dTomato were grown towards 70% confluency, at which point the media was switched to a-MEM with 15% FBS and 1% Pen-Strep to condition the supernatant for 48 h before transducing OP9-DL4-7FS/T-cell cultures. On D8+24 of OP9-DL4-7FS/T-cell cultures, cells were transduced with corresponding PG13 supernatants once a day with 1 μl/ml polybrene and centrifuged at 2000×g for 90 min at RT for 4 d (2-3×10⁶ cells per 2 ml of supe). Cells were rested for 3 d and then sorted as dTomato+CD7+CD5+CD4+CD8+ DPs. Sorted cells were placed in OP9-DL4-7FS co-cultures for up to 10 days.

Flow Cytometry. Cells were stained for 30 min on ice with the following mouse anti-human antibodies: CD3-BrilliantViolet421, CD4-AlexaFluor700, CD8B-PE, CD31-FITC, CD34-PE, CD45RA-PE/CF594, TCRgd-FITC (BD Biosciences), CD5-PE/Cy7, CD7-AlexaFluor700, CD45-APC/eFluor780, TCRab-APC (eBiosciences), CD8a-PE/Dazzle, CD38-BrilliantViolet421 (BioLegend). Cells were resuspended in flow cytometry buffer containing DAPI, data was collected using LSR Fortessa flow cytometer (BD Biosciences) and analyzed using FlowJo version 9.7.6. For intracellular staining, cells were fixed and permeabilized using Fixation/Permeabilization kit with GolgiPlug™ (BD Biosciences) as per manufacturer's instructions.

RNA-Seq Analysis of Control and RAG2-KO in vitro Derived CD4+CD8+ cells. Cells were collected at co-culture day 24-28 and stained with fluorochrome-labelled antibodies to CD45, CD7 (eBioscience), CD5, CD4, CD8 (BD Biosciences), DAPI, and sorted into CD4+CD8+ populations using FACSVantage Diva or FACSAria cell sorters (BD Biosciences). Total RNA was extracted from the sorted cell populations using TRIzol. Purified RNA was subjected to RNA sequencing using Illumina Novaseq 6000. Library preparation was done using Illumina TruSeq Strandard Total RNA Sample Preparation kit. Sequencing was done using 100-cycle paired read protocol and multiplexing to obtain ˜40 million reads/sample. Samples were aligned to GRCh38 using HISAT2. Read counts were calculated using HTSeq. Differential gene expression analysis was done using edgeR package in the R platform.

Statistical Analysis. The data and error bars are presented as mean±standard error of mean. To determine statistical significance, a one-way ANOVA (comparing three means) was performed using Prism version 6. Statistical significance was determined as *P<0.05 and ** P<0.01.

Data Availability. Raw and processed RNA-Seq data are available from the Gene Expression Omnibus under accession number GSE164276 (https://www.ncbi.nlm.nih.gov/geo/query/acc.cgi?acc=GSE164276).

### Results and Discussion

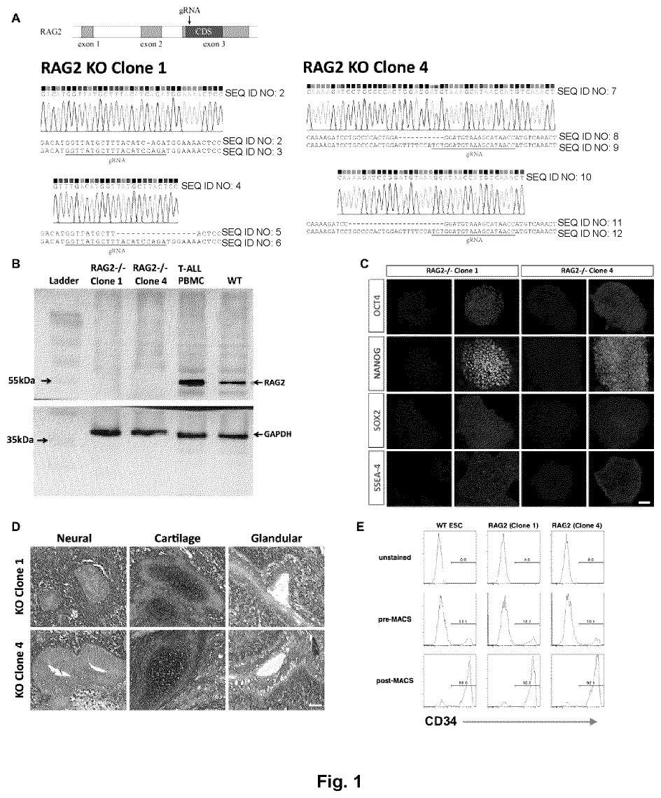

Generation and characterization of RAG2-KO hPSC lines. To evaluate the role of RAG2 in human T-cell development, CRISPR-Cas9 gene editing was used to target exon 3 of the RAG2 gene (FIG. 1A). hPSCs were transfected with a plasmid encoding the RAG2-targeting guide RNA, the Cas9 enzyme, and green fluorescent protein (GFP). Transfected GFP+ hPSCs were single-cell sorted and cultured. After expanding individual clones, two clones were identified that contained unique insertion-deletions with bi-allelic mutations (KO-1 and KO-4) (FIG. 1A). To evaluate the impact of the RAG2 mutations on protein expression, Western blot analysis was performed, which confirmed the absence of detectable RAG2 protein in both KO-1 and -4 derived T-lineage cells (FIG. 1B).

To assess whether RAG2-KO hPSCs maintained pluripotency, the expression of key markers and teratoma formation was evaluated. Immunofluorescence staining showed that RAG2-KO hPSCs expressed OCT4, NANOG, SOX2, and SSEA-4 (FIG. 1C). To functionally test pluripotency, RAG2-KO hPSCs were injected into immunodeficient mice, and histological analysis revealed RAG2-KO teratoma formation with all three germ layers (FIG. 1D), indicating that RAG2-KO hPSCs retained key features of pluripotency. To determine the capacity of RAG2-KO hESCs to generate hematopoietic progenitors, CD34 expression was analyzed after 8 days of embryoid-body differentiation cultures (Kennedy et al, 2012). Control WT, RAG2-KO-1 and -4 hPSCs gave rise to similar frequencies of hemogenic endothelial CD34+ cells, which could be further enriched by magnetic-assisted cell sorting (MACS) (FIG. 1E).

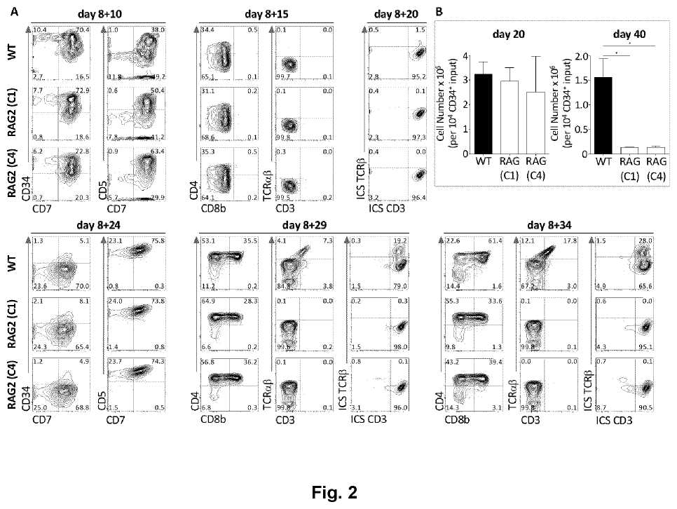

T-cell development from RAG2-KO hPSCs. CD34+ hemogenic endothelial cells were MACS-enriched and cultured with OP9-DL4 cells, expressing human IL-7, FLT3-ligand and stem cell factor (7FS), to induce T-cell differentiation. After 10 days of culture, Control WT, RAG2-KO-1 and -4 cells proceeded along the T-cell lineage, as marked by expression of both CD7 and CD5 (FIG. 2A). All three groups reached the CD4+ intermediate single positive (ISP) stage by day 15 and displayed intracellular CD3 expression by day 20 (FIG. 2A). After 24 days of culture, the majority of cells from Control WT, RAG2-KO-1 and -4 groups were CD7+CD5* (FIG. 2A). Of note, by 29-34 days of culture, Control WT, RAG2-KO-1 and -4 cells all reached the CD4+CD8+DP stage (FIG. 2A). However, as expected, only Control WT cells displayed intracellular TCRβ expression, and cell surface CD3/TCRβ expression (FIG. 2A). These results indicate that RAG2-deficient human T-cell progenitors can differentiate up to the CD4+CD8+DP stage.

To determine cell survival and expansion, total cellularity was quantified from Control WT, RAG2-KO-1 and -4 developing T-cells. After 20 days of culture, all three samples showed similar cell numbers (FIG. 2B). However, after 40 days of culture, Control WT T-lineage cells further increased their survival and expansion as opposed to RAG2-KO-1 and -4 T-lineage cells, with approximately 10-fold greater cellularity by this time-point (FIG. 2B).

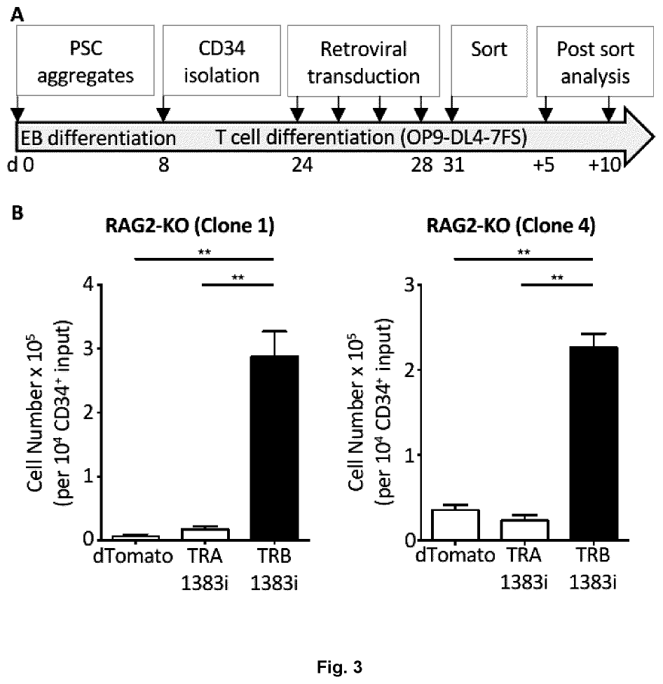

Forced expression of a TCRβ chain in RAG2-KO CD4+CD8+ DPs. RAG2-KO hPSC-derived CD34+ cells were cultured on OP9-DL4-7FS cells for 24 days, and DP cells were retrovirally-transduced with an empty vector (dTomato), a rearranged TCRox chain (TRA 1383i), or a rearranged TCRβ chain (TRB 1383i) (FIG. 3A).

Transduced RAG-KO cells were sorted for CD7+CD5+CD4+CD8+DP cells, and placed back on OP9-DL4-7FS cells for an additional 10 days to assess for cell survival and expansion (FIG. 3A). Both dTomato- and TCRα-transduced DPs showed similar cell numbers after 10 days of culture (FIG. 3B). However, TCRβ-transduced DPs displayed significantly higher cell numbers after 10 days of culture compared to dTomato- and TCRox-transduced DPs (FIG. 3B). Without being bound by theory, this suggests that, in contrast to a TCRα chain, expression of a rearranged TCRβ chain promotes cell survival and/or proliferation of developing human T-cells at the DP stage.

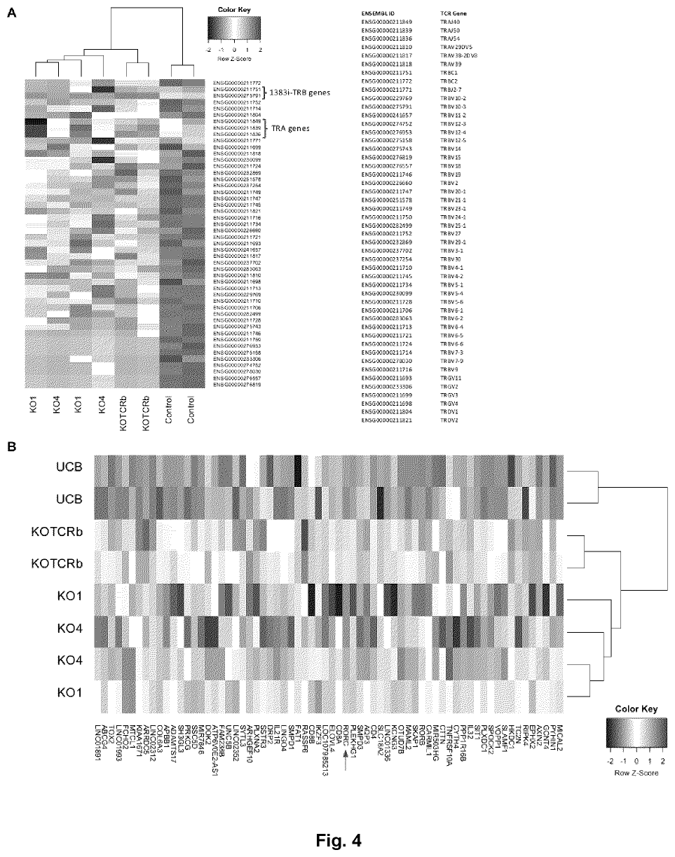

Transcriptomic analysis of Control WT, RAG2-KO, and RAG2-KO TCRβ-transduced CD4+CD8+DP cells. Control WT, RAG2-KO control-transduced, and RAG2-KO TCRβ-transduced DP cells were sorted for RNA sequencing (RNA-Seq) analysis. Control WT DP cells expressed a large set of TCRα, TCRβ, TCRγ, and TCRβ genes that were absent in RAG2-KO control-transduced and RAG-KO TCRβ-transduced DP cells, with the notable exception of some TCR genes, including the 1383i TCRβ used in the transduction and a few TCRα genes, likely the result of germline transcripts induced by the B-selection signals, as seen in mice (Villey et al, 1997) (Table 1, and FIG. 4A). Furthermore, the expression of genes from RAG2-KO control- and TCRβ-transduced DP cells were compared with that of umbilical cord-blood (UCB)-hematopoietic stem cell-derived DP cells for a set of DP-associated signature genes (Casero et al, 2015) (Table 1). This analysis revealed that TCRβ-transduced RAG2-KO DP cells gained the expression of a suite of genes that were present in the UCB-derived DP cells, including RORC, which is induced following β-selection in mice (He 2000) (FIG. 4B)

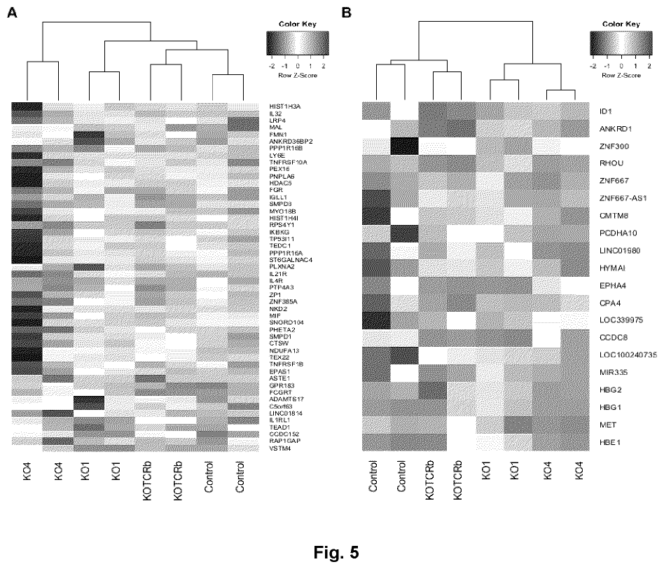

Differentially expressed gene analysis was performed to determine genes that were up-regulated in Control WT compared to RAG2-KO (KO-1 and KO-4) DP cells, and vice versa. Genes significantly up-regulated in Control WT compared to both RAG2-KO-1 and RAG2-KO-4 include CCDCl52, GPR183, IL32, and MAL (Table 1 and FIG. 5A). Genes significantly up-regulated (p<0.05) in Control WT compared to RAG2-KO-1 or -4 included ADAMTS17, IL1RL1, PLXNA2, and TEAD1, or CTSW, IKBKG, IL21R, and IL4R, respectively (Table 1 and FIG. 5A). Of note, many of these genes (such as TEAD1, IKBKG, and IL32) were also highly expressed in TCRβ-transduced RAG2-KO DP cells similar to Control WT DP cells (FIG. 5A). Interestingly, the expression of a subset of genes (such as MAL) were not rescued with forced expression of a TCRβ chain (FIG. 5A). Genes significantly up-regulated in both RAG2-KO-1 and RAG2-KO-4 compared to Control WT include HBG1, HBG2, LOC100240735, and LOC339975 (Table 1 and FIG. 5B). Genes significantly up-regulated in RAG2-KO-1 or -4 compared to Control WT include CCDC8, CPA4, EPHA4, and ID1, or ANKRD1, CMTM8, MET, and RHOU, respectively (Table 1 and FIG. 5B). Of note, many of these genes (such as ID1, RHOU, and HBG1) also showed low expression in TCRβ-transduced RAG2-KO DPs similar to Control WT DP cells (FIG. 5B). Interestingly, the expression of a subset of genes (such as LOC100240735) were not reduced with forced expression of a TCRβ chain (FIG. 5B). Phylogenetic tree analysis again showed that TCRβ-transduced RAG2-KO DP cells were more similar to Control WT DP cells than to control-transduced RAG2-KO DP cells (FIG. 5).

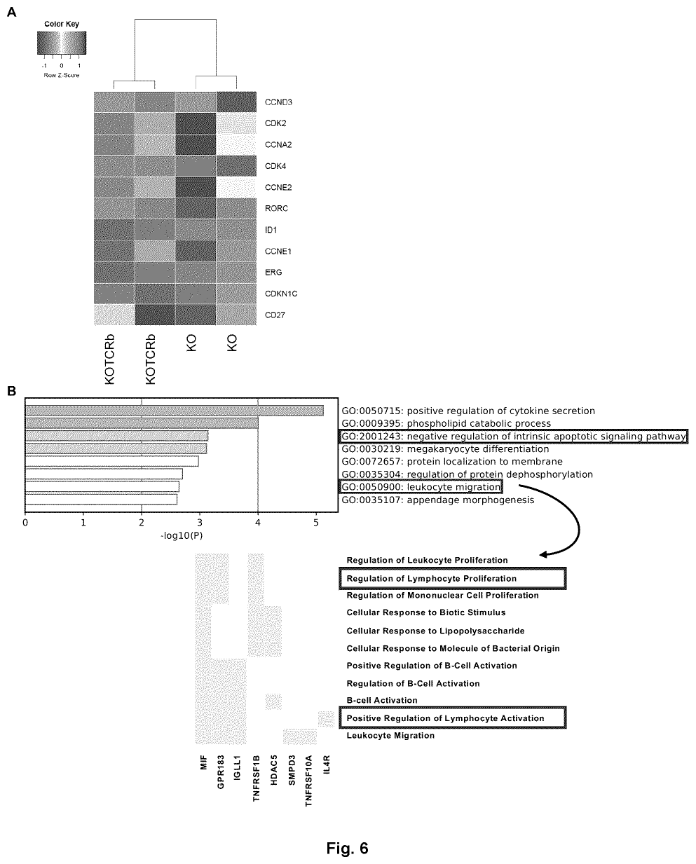

Analysis of cell cycle regulators, survival and differentiation genes, which are known to be involved in mouse B-selection (Lefebvre et al, 2005; Klein et al, 2019; Sicinska et al, 2003), revealed a set of genes, such as RORC, CD27, ERG and CCDN3, that are also regulated following TCRβ expression in RAG2-KO DPs (FIG. 6A). A gene ontology analysis to determine biological pathways that involve genes up-regulated in Control WT compared to RAG2-KO DP cells revealed a genetic program involved in “negative regulation of intrinsic apoptotic signaling pathway” (FIG. 6B). Furthermore, a gene set pathway pertaining to leukocyte regulation, included genes involved in “positive regulation of lymphocyte activation” and “regulation of lymphocyte proliferation” (FIG. 6B). Thus, these data provide additional evidence that RAG2-dependent TCRβ expression in developing human T-cells supports their survival and/or proliferation.

### SUMMARY

RAG2-KO hPSCs were generated to assess the role of TCRβ during human T-cell development. In contrast to the effects seen with Rag1- or Rag2-deficient mice, which show a complete lack of DP thymocytes, developing human T-cells were able to reach the CD4+CD8+DP stage in the absence of RAG2 expression. Lack of RAG1/2 in mice results in a definitive block at the CD44 CD25+DN3 stage, as it is well documented that RAG1/2-mediated TCRβ rearrangement controls the transition from the DN3 to the CD44 CD25 DN4 and DP stages (von Boehmer et al, 1999; Michie and Zuniga-Pflucker, 2002). However, the precise developmental block in the absence of RAG1/2 expression or TCRβ rearrangement during human T-cell development was largely unresolved (Rothenberg and Taghon, 2005; Carrasco et al, 2002).

It was previously suggested that the requirement for TCRβ-induced survival/proliferation, or -selection, occurs at the CD4+ ISP stage, in which 5% of cells express cell-surface TCRβ protein (Blom et al, 1999). In another study, it was suggested that B-selection instead happens later, at the CD4+CD8α+CD8β early double positive (EDP) stage in which 25% of cells express intracellular TCRβ protein (Carrasco et al, 1999). Thus, based on these two studies, it was proposed that B-selection begins at the CD4+ ISP stage and continues until the EDP stage. Here, robust development was detected past the ISP and EDP to the DP stage, as CD4, CD8α, and CD8β expression was observed in RAG2-KO developing T-cells. While the requirement for the TCRβ chain in the phenotypic differentiation to the DP stage differs between mice and humans, the TCRβ chain similarly promotes cellular expansion (survival/proliferation) in both mouse and human developing T-cells, as enforced TCRβ expression rescued cellular expansion. Without being bound by theory, the requirement for TCRβ, and thus the preTCR, during, rather than prior to, the DP stage during human T-cell development may reflect differential expression of cell cycle and survival genes. In mice, expression of a preTCR in DN3 cells leads to expression of Bmi-1, which represses the expression of the cell cycle inhibitor Cdkn2a. Repression of Cdkn2a is required for preTCR-induced cell proliferation and the DN3-DP transition (Miyazaki et al, 2008). In summary, this study reveals the unexpected timing required for TCRβ-mediated B-selection in developing human T-cells.

### Example 2

Forced expression of a TCRαβ and TCRγδ chains in RAG2-KO CD4+CD8+ DPs

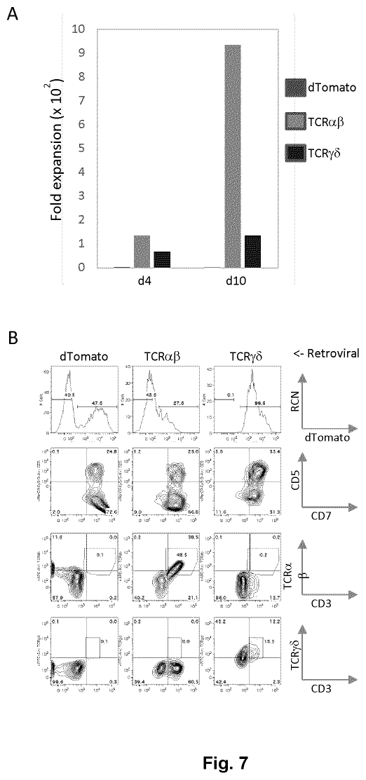

RAG2-KO hPSC-derived CD34+ cells were cultured on OP9-DL4-7FS cells for 21 days, and DP cells were retrovirally-transduced with an empty vector (dTomato), rearranged TCRαβ chains (1383i-TCR) (Roszkowski et al., 2003), or rearranged TCRγδ chains (3C2-TCR) (Benveniste et al., 2018) (FIG. 7). Transduced RAG-KO cells were sorted for CD7+CD5+CD4+CD8+DP cells, and placed back on OP9-DL4-7FS cells for an additional 4 and 10 days to assess for cell survival and expansion (FIG. 7A). TCRαβ- and TCRγδ-transduced DPs displayed higher cell numbers after 4 and 10 days of culture compared to dTomato-transduced DPs (FIG. 7A), with TCRαβ-transduced cells showing a much higher fold expansion than TCRγδ-transduced cells, and akin to what was seen with TCRβ-transduced DP cells (FIG. 3). Without being bound by theory, this suggests that similar to a TCRβ chain alone (preTCR, pTα/TCRβ), expression of a rearranged TCRαβ or TCRγδ chains promote cell survival and/or proliferation of developing human T-cells at the DP stage. Additionally, flow cytometric analysis of TCR-transduced RAG2-KO DP cells showed the expression of the corresponding αβ and γδ TCRs, respectively, on the cell surface of T-lineage cells (FIG. 7B). These results show that developing T cells obtained from RAG2-deficient PSCs differentiated in vitro can be transduced to effectively express a γδ or αβ TCR.

### Example 3

CRISPR-Cas9 gene editing is used to target each of the following genes: Artemis, DNA-dependent protein kinase (DNA-PK), X-ray repair cross-complementing protein 4 (XRCC4), DNA ligase IV, non-homologous end-joining factor 1 (NHEJ1; also known as Cernunnos or XRCC4-like factor (XLF)), Paralog of XRCC4 and XLF (PAXX), DNA polymerase λ and DNA polymerase μ.

hPSCs are transfected with a plasmid encoding the gene targeting guide RNA, the Cas9 enzyme, and green fluorescent protein (GFP). Transfected GFP+ hPSCs are single-cell sorted and cultured.

To assess whether various KO hPSCs maintained pluripotency, the expression of key markers and teratoma formation are evaluated. Immunofluorescence staining shows that the KO hPSCs express OCT4, NANOG, SOX2, and SSEA-4.

To functionally test pluripotency, the KO hPSCs are injected into immunodeficient mice, and histological analysis reveals KO teratoma formation with all three germ layers, indicating that KO hPSCs retain key features of pluripotency. To determine the capacity of KO hESCs to generate hematopoietic progenitors, CD34 expression is analyzed after 8 days of embryoid-body differentiation cultures (Kennedy et al, 2012). Control WT and KO hPSCs give rise to similar frequencies of hemogenic endothelial CD34+ cells.

T-cell development from KO hPSCs. CD34+ hemogenic endothelial cells are MACS-enriched and cultured with OP9-DL4 cells, expressing human IL-7, FLT3-ligand and stem cell factor (7FS), to induce T-cell differentiation. After 10 days of culture, Control WT and KO cells proceed along the T-cell lineage, as marked by expression of both CD7 and CD5. All three groups reach the CD4+ intermediate single positive (ISP) stage by day 15 and display intracellular CD3 expression by day 20. After 24 days of culture, the majority of cells from Control WT and KO groups are CD7+CD5+ By 29-34 days of culture, Control WT and KO cells all reach the CD4+CD8+DP stage.

To determine cell survival and expansion, total cellularity is quantified from Control WT, and KO developing T-cells. After 20 days of culture, all three samples show similar cell numbers.

Forced expression of a TCRβ chain in KO CD4+CD8+ DPs. KO hPSC-derived CD34+ cells are cultured on OP9-DL4-7FS cells for 24 days, and DP cells are retrovirally-transduced with an empty vector (dTomato), a rearranged TCRα chain (TRA 1383i), or a rearranged TCRβ chain (TRB 1383i). Transduced KO cells are sorted for CD7+CD5+CD4+CD8+DP cells, and placed back on OP9-DL4-7FS cells for an additional days to assess for cell survival and expansion. Both dTomato- and TCRα-transduced DPs show similar cell numbers after 10 days of culture. However, TCRβ-transduced DPs display significantly higher cell numbers after 10 days of culture compared to dTomato- and TCRα-transduced DPs.

### Example 4

### Generation of KO SP T Cells

Generation of KO SP T cells. KO hPSCs engineered with one or more of a rearranged TCRβ chain (TRB 1383i), a CAR or a TCR are differentiated into CD34+ cells and subsequently CD4+CD8+DP cells as above or in stromal cell-free conditions (see for example WO2019157597A1, the contents of which are incorporated by reference in their entirety). Generated CD4+CD8+DP cells are further differentiated into CD4-CD8+SP and/or CD8-CD4+SP T cells. KO DP cells engineered with TCRβ successfully progress to SP T cells in comparison with KO-only cells.

TABLE 1
List of genes significantly up-regulated (p < 0.05) in Control WT DPs compared
to RAG2-KO-1 DPs, or vice versa, as indicated: (NA: non-assigned TCR gene names).

| Up in Control | | | | | |
|---|---|---|---|---|---|
| ENSEMBL | SYMBOL | LogFC | Ave Exp | t Value | p Value |
| ENSG00000198865 | CCDC152 | 8.517702 | −1.2060943 | 8.196432 | 2.4619E−05 |
| ENSG00000211750 | <NA> | 8.318697 | −2.8399921 | 12.630925 | 1.0213E−06 |
| ENSG00000211746 | <NA> | 7.923854 | −2.6560982 | 8.493945 | 1.8718E−05 |
| ENSG00000276953 | <NA> | 7.718605 | −2.9993099 | 8.146927 | 2.5048E−05 |
| ENSG00000275158 | <NA> | 7.319261 | −3.0939512 | 12.344204 | 1.1756E−06 |
| ENSG00000274752 | <NA> | 6.821362 | −3.0215316 | 8.377361 | 1.9917E−05 |
| ENSG00000278030 | <NA> | 6.732332 | −3.0446835 | 7.923013 | 2.9525E−05 |
| ENSG00000211706 | <NA> | 6.633362 | −1.9248385 | 8.05775 | 2.611E−05 |
| ENSG00000282499 | <NA> | 6.417086 | −2.0223909 | 6.873888 | 7.8658E−05 |
| ENSG00000276557 | <NA> | 6.368357 | −3.3316934 | 10.397811 | 4.0823E−06 |
| ENSG00000211693 | <NA> | 6.307571 | −1.3708525 | 5.433344 | 0.00037427 |
| ENSG00000275791 | <NA> | 6.191937 | 1.60630085 | 9.452939 | 8.1556E−06 |
| ENSG00000275743 | <NA> | 6.189794 | −2.5862869 | 6.486807 | 0.00011582 |
| ENSG00000241657 | <NA> | 6.073368 | −2.4291257 | 9.598497 | 7.2677E−06 |
| ENSG00000211721 | <NA> | 6.02149 | −0.7628611 | 9.334584 | 8.888E−06 |
| ENSG00000211710 | <NA> | 5.894499 | −1.981085 | 7.189231 | 5.6671E−05 |
| ENSG00000211728 | <NA> | 5.821217 | −2.5371219 | 5.304768 | 0.00042805 |
| ENSG00000233306 | <NA> | 5.820969 | −3.0621388 | 4.320709 | 0.00149548 |
| ENSG00000211745 | <NA> | 5.516755 | −0.1808708 | 9.413935 | 8.2219E−06 |
| ENSG00000276819 | <NA> | 5.461533 | −3.5593405 | 8.314578 | 2.0077E−05 |
| ENSG00000230006 | ANKRD36BP2 | 5.414753 | 3.27069758 | 4.145606 | 0.00186578 |
| ENSG00000211821 | <NA> | 5.360107 | −0.3451044 | 5.975287 | 0.00019464 |
| ENSG00000283063 | <NA> | 5.088158 | −2.876393 | 5.022423 | 0.00058681 |
| ENSG00000211734 | <NA> | 5.056142 | −0.468777 | 7.965634 | 2.6874E−05 |
| ENSG00000237702 | <NA> | 5.02606 | −2.6835032 | 4.99639 | 0.0006046 |
| ENSG00000172005 | MAL | 4.956678 | 4.04116362 | 4.478536 | 0.00116925 |
| ENSG00000211747 | <NA> | 4.852242 | −0.0395991 | 9.746459 | 6.2572E−06 |
| ENSG00000211698 | <NA> | 4.674811 | −2.0689517 | 4.365069 | 0.00134415 |
| ENSG00000211817 | <NA> | 4.569333 | −2.7916217 | 4.999176 | 0.00059255 |
| ENSG00000226660 | <NA> | 4.568507 | −1.5216111 | 3.92235 | 0.00245463 |
| ENSG00000164241 | C5orf63 | 4.412744 | −1.2728461 | 2.98561 | 0.00972973 |
| ENSG00000140470 | ADAMTS17 | 4.114259 | −0.2748657 | 2.257093 | 0.03045552 |
| ENSG00000211810 | <NA> | 4.016163 | −2.6681461 | 3.632342 | 0.00362759 |
| ENSG00000211713 | <NA> | 3.765733 | −2.1670369 | 4.191198 | 0.00164333 |
| ENSG00000165633 | VSTM4 | 3.717044 | −2.2032977 | 2.036388 | 0.04241983 |
| ENSG00000211849 | <NA> | 3.620071 | −1.1571862 | 2.76704 | 0.01311932 |
| ENSG00000232869 | <NA> | 3.48762 | −0.5815655 | 4.649635 | 0.00089024 |
| ENSG00000210194 | <NA> | 3.460159 | −0.9030918 | 2.211914 | 0.03135952 |
| ENSG00000211716 | <NA> | 3.365599 | −0.2876694 | 4.592905 | 0.00095486 |
| ENSG00000237254 | <NA> | 3.290203 | −0.466529 | 4.784741 | 0.00074607 |
| ENSG00000230099 | <NA> | 3.288033 | −0.8427471 | 2.640981 | 0.01571927 |
| ENSG00000251578 | <NA> | 3.224662 | −1.0269264 | 3.405065 | 0.00490892 |
| ENSG00000211749 | <NA> | 3.209309 | −0.6280164 | 3.53171 | 0.00407815 |
| ENSG00000211714 | <NA> | 3.100552 | 0.63664893 | 3.52919 | 0.00408052 |
| ENSG00000115602 | IL1RL1 | 2.918121 | −0.6201869 | 3.231124 | 0.00629336 |
| ENSG00000211724 | <NA> | 2.917557 | −0.9573169 | 2.920958 | 0.01004788 |
| ENSG00000229769 | <NA> | 2.873665 | −2.2358285 | 3.649591 | 0.00341216 |
| ENSG00000076356 | PLXNA2 | 2.779821 | 1.2204853 | 2.462547 | 0.02032877 |
| ENSG00000248905 | FMN1 | 2.680991 | 3.55553195 | 3.328335 | 0.0054196 |
| ENSG00000211818 | <NA> | 2.678134 | −1.178139 | 2.833739 | 0.01140162 |
| ENSG00000211752 | <NA> | 2.621173 | 0.71807768 | 4.534972 | 0.00101587 |
| ENSG00000187079 | TEAD1 | 2.398258 | −0.6408802 | 1.898367 | 0.04842699 |
| ENSG00000169508 | GPR183 | 2.395666 | −0.0382727 | 1.901482 | 0.04818661 |
| ENSG00000211804 | <NA> | 2.276024 | 0.17880719 | 3.010357 | 0.00865148 |
| ENSG00000211836 | <NA> | 2.200166 | −1.0795964 | 1.965313 | 0.04330296 |
| ENSG00000211839 | <NA> | 2.166826 | −0.5365795 | 2.052539 | 0.03778579 |
| ENSG00000008517 | IL32 | 1.846931 | 4.18320491 | 2.442861 | 0.02053788 |
| ENSG00000211772 | <NA> | 1.539048 | 7.83477128 | 3.108563 | 0.00743986 |

TABLE 2
List of genes significantly up-regulated (p < 0.05) in Control WT DPs compared
to RAG2-KO-4 DPs, or vice versa, as indicated: (NA: non-assigned TCR gene names).

| Up in Control | | | | | |
|---|---|---|---|---|---|
| ENSEMBL | SYMBOL | LogFC | Ave Exp | t Value | p Value |
| ENSG00000211750 | <NA> | 8.162062 | −2.8399921 | 12.360598 | 1.1956E−06 |
| ENSG00000211746 | <NA> | 7.767219 | −2.6560982 | 8.30179 | 2.1936E−05 |
| ENSG00000276953 | <NA> | 7.56197 | −2.9993099 | 7.956993 | 2.9463E−05 |
| ENSG00000211734 | <NA> | 7.208251 | −0.468777 | 9.045181 | 1.162E−05 |
| ENSG00000275158 | <NA> | 7.162626 | −3.0939512 | 12.038229 | 1.4115E−06 |
| ENSG00000034533 | ASTE1 | 6.972768 | 0.67498444 | 2.250502 | 0.03632517 |
| ENSG00000211728 | <NA> | 6.825546 | −2.5371219 | 6.409828 | 0.00012845 |
| ENSG00000211710 | <NA> | 6.51719 | −1.981085 | 7.584541 | 3.9866E−05 |
| ENSG00000233306 | <NA> | 6.456816 | −3.0621388 | 4.890575 | 0.00072978 |
| ENSG00000276557 | <NA> | 6.211721 | −3.3316934 | 10.09443 | 5.0497E−06 |
| ENSG00000283063 | <NA> | 6.092487 | −2.876393 | 6.256271 | 0.00014724 |
| ENSG00000275743 | <NA> | 6.033159 | −2.5862869 | 6.291026 | 0.00014157 |
| ENSG00000274752 | <NA> | 5.872245 | −3.0215316 | 7.011514 | 6.7324E−05 |
| ENSG00000278030 | <NA> | 5.783216 | −3.0446835 | 6.61118 | 0.00010036 |
| ENSG00000211706 | <NA> | 5.711686 | −1.9248385 | 6.987882 | 6.8525E−05 |
| ENSG00000275791 | <NA> | 5.601616 | 1.60630085 | 8.856185 | 1.2829E−05 |
| ENSG00000282499 | <NA> | 5.589299 | −2.0223909 | 5.964671 | 0.00019857 |
| ENSG00000198865 | CCDC152 | 5.586937 | −1.2060943 | 6.027127 | 0.00018534 |
| ENSG00000226660 | <NA> | 5.579945 | −1.5216111 | 4.410879 | 0.00131195 |
| ENSG00000129824 | RPS4Y1 | 5.458735 | 5.87336909 | 2.187663 | 0.03724085 |
| ENSG00000211747 | <NA> | 5.332693 | −0.0395991 | 10.173377 | 4.6369E−06 |
| ENSG00000211693 | <NA> | 5.316543 | −1.3708525 | 4.887327 | 0.00070055 |
| ENSG00000276819 | <NA> | 5.304898 | −3.5593405 | 8.022671 | 2.5753E−05 |
| ENSG00000211810 | <NA> | 5.276514 | −2.6681461 | 5.123064 | 0.00052226 |
| ENSG00000211821 | <NA> | 5.247636 | −0.3451044 | 5.816677 | 0.00023135 |
| ENSG00000237702 | <NA> | 4.869425 | −2.6835032 | 4.802004 | 0.00076692 |
| ENSG00000211745 | <NA> | 4.844219 | −0.1808708 | 8.491467 | 1.6946E−05 |
| ENSG00000211721 | <NA> | 4.78909 | −0.7628611 | 8.034847 | 2.5073E−05 |
| ENSG00000211716 | <NA> | 4.72396 | −0.2876694 | 6.054901 | 0.00017449 |
| ENSG00000211698 | <NA> | 4.545294 | −2.0689517 | 4.243552 | 0.00157464 |
| ENSG00000229769 | <NA> | 4.491292 | −2.2358285 | 5.949991 | 0.00019436 |
| ENSG00000232869 | <NA> | 4.451973 | −0.5815655 | 5.683435 | 0.00026184 |
| ENSG00000211713 | <NA> | 4.38113 | −2.1670369 | 4.823196 | 0.00073347 |
| ENSG00000116016 | EPAS1 | 4.295392 | −0.8807061 | 4.713222 | 0.00084097 |
| ENSG00000251578 | <NA> | 4.178802 | −1.0269264 | 4.274236 | 0.00148993 |
| ENSG00000230099 | <NA> | 4.042578 | −0.8427471 | 3.100613 | 0.00800344 |
| ENSG00000076864 | RAP1GAP | 3.959056 | −1.7469106 | 2.664417 | 0.01569386 |
| ENSG00000172005 | MAL | 3.95788 | 4.04116362 | 3.625373 | 0.00365575 |
| ENSG00000237254 | <NA> | 3.856401 | −0.466529 | 5.495478 | 0.00031948 |
| ENSG00000028137 | TNFRSF1B | 3.79933 | −0.6752704 | 2.033321 | 0.04289521 |
| ENSG00000145506 | NKD2 | 3.757573 | 0.27551971 | 2.394666 | 0.02383015 |
| ENSG00000277048 | <NA> | 3.741285 | 1.44983714 | 2.310452 | 0.02725463 |
| ENSG00000211817 | <NA> | 3.647657 | −2.7916217 | 3.832094 | 0.0026902 |
| ENSG00000241657 | <NA> | 3.627882 | −2.4291257 | 5.573106 | 0.00029035 |
| ENSG00000211714 | <NA> | 3.616438 | 0.63664893 | 4.073116 | 0.00192154 |
| ENSG00000008517 | IL32 | 3.284801 | 4.18320491 | 5.796536 | 0.0002236 |
| ENSG00000211771 | <NA> | 3.268893 | −1.512696 | 2.044763 | 0.04050564 |
| ENSG00000211749 | <NA> | 3.198221 | 0.6280164 | 3.451613 | 0.0045814 |
| ENSG00000177096 | PHETA2 | 3.152482 | 0.13121737 | 3.825269 | 0.00267658 |
| ENSG00000226174 | TEX22 | 3.002381 | −0.5562848 | 2.246348 | 0.0288737 |
| ENSG00000169508 | GPR183 | 2.99354 | −0.0382727 | 2.515646 | 0.01887334 |
| ENSG00000274452 | <NA> | 2.97966 | −0.9626708 | 2.031624 | 0.04056901 |
| ENSG00000211724 | <NA> | 2.902091 | −0.9573169 | 2.850146 | 0.01119255 |
| ENSG00000199753 | SNORD104 | 2.896863 | −0.0738509 | 2.107037 | 0.0358039 |
| ENSG00000104870 | FCGRT | 2.881885 | −0.2470579 | 3.283793 | 0.00581431 |
| ENSG00000077238 | IL4R | 2.86209 | 0.37944373 | 2.434344 | 0.0213187 |
| ENSG00000211818 | <NA> | 2.861742 | −1.178139 | 3.042362 | 0.00834138 |
| ENSG00000211752 | <NA> | 2.859241 | 0.71807768 | 5.020002 | 0.00055247 |
| ENSG00000276180 | HIST1H4I | 2.848206 | 5.47377595 | 1.965865 | 0.04466518 |
| ENSG00000149506 | ZP1 | 2.788498 | 0.14468104 | 2.009078 | 0.04155506 |
| ENSG00000240972 | MIF | 2.76273 | 0.26136195 | 2.784547 | 0.01232517 |
| ENSG00000172543 | CTSW | 2.705115 | 0.14611252 | 3.315826 | 0.00552314 |
| ENSG00000136840 | ST6GALNAC4 | 2.702985 | 1.71210383 | 2.035465 | 0.03966518 |
| ENSG00000184489 | PTP4A3 | 2.649319 | 0.22426429 | 1.923271 | 0.04719826 |
| ENSG00000032444 | PNPLA6 | 2.618052 | 2.52046666 | 2.051902 | 0.03848134 |
| ENSG00000160932 | LY6E | 2.611928 | 3.19636005 | 2.56894 | 0.01712299 |
| ENSG00000166311 | SMPD1 | 2.521156 | 0.20609228 | 2.363001 | 0.02353312 |
| ENSG00000239791 | <NA> | 2.487449 | −0.4539044 | 2.619593 | 0.01577698 |
| ENSG00000185347 | TEDC1 | 2.478509 | 1.29168656 | 1.892602 | 0.0490595 |
| ENSG00000103522 | IL21R | 2.403267 | 0.84514527 | 3.501089 | 0.00419294 |
| ENSG00000160972 | PPP1R16A | 2.334936 | 1.11911499 | 2.063406 | 0.0373436 |
| ENSG00000128322 | IGLL1 | 2.316112 | 2.83224971 | 2.871366 | 0.01068525 |
| ENSG00000186010 | NDUFA13 | 2.300787 | −0.2558601 | 1.911583 | 0.04723936 |
| ENSG00000211699 | <NA> | 2.295547 | −0.6068566 | 2.04705 | 0.03825609 |
| ENSG00000161642 | ZNF385A | 2.292218 | −0.1031169 | 1.877838 | 0.04976267 |
| ENSG00000101445 | PPP1R16B | 2.283182 | 3.29912799 | 2.629054 | 0.01548246 |
| ENSG00000275714 | HIST1H3A | 2.250361 | 4.48007601 | 1.874151 | 0.04996283 |
| ENSG00000271869 | <NA> | 2.242191 | −0.527668 | 2.101436 | 0.03509275 |
| ENSG00000121680 | PEX16 | 2.241312 | 2.55954191 | 2.392867 | 0.02229991 |
| ENSG00000175274 | TP53I11 | 2.143382 | 1.31932639 | 2.058014 | 0.03744372 |
| ENSG00000279693 | <NA> | 2.131329 | 2.43865387 | 2.582929 | 0.01658628 |
| ENSG00000000938 | FGR | 2.105363 | 2.03569751 | 1.887195 | 0.04872219 |
| ENSG00000108840 | HDAC5 | 2.074299 | 2.24683163 | 1.890163 | 0.04845715 |
| ENSG00000134569 | LRP4 | 2.01582 | 3.88594501 | 1.921207 | 0.04612707 |
| ENSG00000133454 | MYO18B | 1.993414 | 5.99665506 | 1.992781 | 0.04128694 |
| ENSG00000236008 | LINC01814 | 1.986033 | −0.7381362 | 1.939974 | 0.04478419 |
| ENSG00000269335 | IKBKG | 1.940037 | 1.51124153 | 1.924645 | 0.04581363 |
| ENSG00000261971 | <NA> | 1.858333 | 0.8888184 | 2.276229 | 0.02658208 |
| ENSG00000104689 | TNFRSF10A | 1.858278 | 2.90340726 | 2.140771 | 0.03278424 |
| ENSG00000211772 | <NA> | 1.844483 | 7.83477128 | 4.823551 | 0.00070023 |
| ENSG00000103056 | SMPD3 | 1.832234 | 6.47443056 | 3.044848 | 0.00818832 |
| ENSG00000211804 | <NA> | 1.774673 | 0.17880719 | 1.895796 | 0.04777756 |
| ENSG00000211751 | <NA> | 1.600269 | 7.81676926 | 2.101263 | 0.03479669 |

TABLE 3
List of genes significantly up-regulated (p < 0.05) in thymus
DNs (CD34+ CD7+ CD1a+ CD4− CD8−) compared
to thymus DPs (CD4+ CD8+), or vice versa from Casero et
al., 2015 (18), as indicated: (NA: non-assigned TCR gene names).

| Up in DN | | | | | |
|---|---|---|---|---|---|
| ENSA167: D207 | SYMBOL | LogFC | AveExp | t Value | p Value |
| ENSG00000157554 | ERG | 9.18E+00 | 2.59E+00 | 7.91E+00 | 7.23E−05 |
| ENSG00000124780 | KCNK17 | 7.98E+00 | 1.20E+00 | 5.36E+00 | 7.17E−04 |
| ENSG00000004809 | SLC22A16 | 7.63E+00 | 2.19E−01 | 6.23E+00 | 2.93E−04 |
| ENSG00000136531 | SCN2A | 7.62E+00 | 2.19E−01 | 6.30E+00 | 2.73E−04 |
| ENSG00000169994 | MYO7B | 7.60E+00 | 2.65E+00 | 4.43E+00 | 2.02E−03 |
| ENSG00000181104 | F2R | 7.47E+00 | 1.30E+00 | 5.42E+00 | 6.58E−04 |
| ENSG00000228065 | <NA> | 7.47E+00 | 1.44E−01 | 6.14E+00 | 3.18E−04 |
| ENSG00000149131 | SERPING1 | 7.41E+00 | 1.05E−01 | 5.78E+00 | 4.53E−04 |
| ENSG00000137868 | STRA6 | 7.41E+00 | 9.05E−01 | 6.11E+00 | 3.27E−04 |
| ENSG00000000971 | CFH | 7.40E+00 | 1.51E+00 | 5.13E+00 | 8.94E−04 |
| ENSG00000132465 | JCHAIN | 7.30E+00 | 1.47E+00 | 4.21E+00 | 2.60E−03 |
| ENSG00000163625 | WDFY3 | 7.26E+00 | 8.12E−01 | 4.34E+00 | 2.23E−03 |
| ENSG00000224739 | <NA> | 7.20E+00 | −2.20E−02 | 4.16E+00 | 2.77E−03 |
| ENSG00000246985 | SOCS2-AS1 | 7.14E+00 | −2.84E−02 | 5.58E+00 | 5.50E−04 |
| ENSG00000115956 | PLEK | 7.04E+00 | −6.62E−02 | 5.57E+00 | 5.52E−04 |
| ENSG00000128322 | IGLL1 | 6.92E+00 | 6.57E−01 | 5.12E+00 | 8.85E−04 |
| ENSG00000116574 | RHOU | 6.85E+00 | 6.24E−01 | 5.55E+00 | 5.59E−04 |
| ENSG00000174059 | CD34 | 6.83E+00 | 2.34E+00 | 6.21E+00 | 2.91E−04 |
| ENSG00000024422 | EHD2 | 6.81E+00 | −1.94E−01 | 5.23E+00 | 7.86E−04 |
| ENSG00000154188 | ANGPT1 | 6.80E+00 | −2.05E−01 | 5.08E+00 | 9.24E−04 |
| ENSG00000251463 | <NA> | 6.79E+00 | −1.93E−01 | 5.44E+00 | 6.28E−04 |
| ENSG00000081189 | MEF2C | 6.78E+00 | 1.39E+00 | 5.49E+00 | 5.92E−04 |
| ENSG00000100320 | RBFOX2 | 6.61E+00 | 1.80E+00 | 3.95E+00 | 3.50E−03 |
| ENSG00000211688 | <NA> | 6.60E+00 | −2.81E−01 | 5.11E+00 | 8.84E−04 |
| ENSG00000113140 | SPARC | 6.57E+00 | −3.00E−01 | 5.15E+00 | 8.46E−04 |
| ENSG00000132464 | ENAM | 6.54E+00 | −3.30E−01 | 5.05E+00 | 9.44E−04 |
| ENSG00000183780 | SLC35F3 | 6.54E+00 | −3.30E−01 | 5.05E+00 | 9.44E−04 |
| ENSG00000211825 | <NA> | 6.53E+00 | −3.24E−01 | 5.18E+00 | 8.25E−04 |
| ENSG00000196460 | RFX8 | 6.50E+00 | 1.96E+00 | 3.80E+00 | 4.24E−03 |
| ENSG00000155254 | MARVELD1 | 6.43E+00 | 4.15E−01 | 5.12E+00 | 8.72E−04 |
| ENSG00000279117 | <NA> | 6.42E+00 | 1.91E+00 | 5.17E+00 | 8.29E−04 |
| ENSG00000100448 | CTSG | 6.34E+00 | −3.95E−01 | 4.01E+00 | 3.22E−03 |
| ENSG00000185215 | TNFAIP2 | 6.28E+00 | 9.81E−01 | 3.04E+00 | 1.19E−02 |
| ENSG00000137731 | FXYD2 | 6.22E+00 | 1.72E+00 | 4.62E+00 | 1.52E−03 |
| ENSG00000271270 | TMCC1-AS1 | 6.19E+00 | 2.78E−01 | 4.15E+00 | 2.70E−03 |
| ENSG00000211695 | <NA> | 6.19E+00 | 1.21E+00 | 3.87E+00 | 3.84E−03 |
| ENSG00000211689 | TARP | 6.13E+00 | 2.75E+00 | 6.04E+00 | 3.34E−04 |
| ENSG00000249100 | <NA> | 6.10E+00 | 2.54E−01 | 4.79E+00 | 1.24E−03 |
| ENSG00000150457 | LATS2 | 6.05E+00 | −5.41E−01 | 3.91E+00 | 3.60E−03 |
| ENSG00000231533 | <NA> | 6.04E+00 | −5.62E−01 | 4.52E+00 | 1.69E−03 |
| ENSG00000224373 | <NA> | 6.01E+00 | −5.90E−01 | 4.62E+00 | 1.51E−03 |
| ENSG00000171502 | COL24A1 | 6.00E+00 | 1.00E+00 | 4.50E+00 | 1.75E−03 |
| ENSG00000272711 | <NA> | 5.97E+00 | −6.09E−01 | 4.58E+00 | 1.59E−03 |
| ENSG00000141431 | ASXL3 | 5.92E+00 | −6.32E−01 | 4.53E+00 | 1.67E−03 |
| ENSG00000120833 | SOCS2 | 5.86E+00 | 2.62E+00 | 5.70E+00 | 4.60E−04 |
| ENSG00000164100 | NDST3 | 5.86E+00 | 4.64E+00 | 5.71E+00 | 4.56E−04 |
| ENSG00000113269 | RNF130 | 5.85E+00 | 1.26E+00 | 4.65E+00 | 1.45E−03 |
| ENSG00000019144 | PHLDB1 | 5.85E+00 | 7.25E−01 | 4.03E+00 | 3.06E−03 |
| ENSG00000223631 | <NA> | 5.82E+00 | −6.92E−01 | 4.28E+00 | 2.24E−03 |
| ENSG00000211692 | <NA> | 5.81E+00 | −7.00E−01 | 4.15E+00 | 2.64E−03 |
| ENSG00000110080 | ST3GAL4 | 5.78E+00 | 8.55E−02 | 4.41E+00 | 1.92E−03 |
| ENSG00000088826 | SMOX | 5.76E+00 | 4.41E−01 | 4.16E+00 | 2.60E−03 |
| ENSG00000053524 | MCF2L2 | 5.76E+00 | 1.46E+00 | 3.30E+00 | 8.09E−03 |
| ENSG00000124570 | SERPINB6 | 5.75E+00 | −7.13E−01 | 4.34E+00 | 2.08E−03 |
| ENSG00000145113 | MUC4 | 5.72E+00 | 8.65E−01 | 4.22E+00 | 2.41E−03 |
| ENSG00000269113 | TRABD2B | 5.72E+00 | −7.17E−01 | 4.08E+00 | 2.87E−03 |
| ENSG00000054793 | ATP9A | 5.70E+00 | 2.82E+00 | 3.79E+00 | 4.15E−03 |
| ENSG00000163106 | HPGDS | 5.68E+00 | 1.47E+00 | 4.28E+00 | 2.22E−03 |
| ENSG00000166265 | CYYR1 | 5.65E+00 | −7.74E−01 | 4.16E+00 | 2.58E−03 |
| ENSG00000260244 | <NA> | 5.64E+00 | 2.19E+00 | 3.37E+00 | 7.32E−03 |
| ENSG00000105655 | ISYNA1 | 5.64E+00 | 6.30E−01 | 4.11E+00 | 2.77E−03 |
| ENSG00000182749 | PAQR7 | 5.63E+00 | −7.72E−01 | 4.23E+00 | 2.37E−03 |
| ENSG00000176641 | RNF152 | 5.60E+00 | 2.00E−02 | 3.61E+00 | 5.27E−03 |
| ENSG00000131969 | ABHD12B | 5.60E+00 | −7.59E−01 | 3.29E+00 | 8.10E−03 |
| ENSG00000101194 | SLC17A9 | 5.59E+00 | −4.23E−03 | 4.27E+00 | 2.26E−03 |
| ENSG00000166482 | MFAP4 | 5.56E+00 | 4.34E+00 | 6.45E+00 | 2.20E−04 |
| ENSG00000115297 | TLX2 | 5.56E+00 | −8.08E−01 | 4.15E+00 | 2.61E−03 |
| ENSG00000148335 | NTMT1 | 5.53E+00 | −8.47E−01 | 3.82E+00 | 3.98E−03 |
| ENSG00000259042 | <NA> | 5.51E+00 | −8.55E−01 | 3.82E+00 | 3.95E−03 |
| ENSG00000248870 | <NA> | 5.50E+00 | 3.10E−01 | 3.92E+00 | 3.46E−03 |
| ENSG00000214711 | CAPN14 | 5.48E+00 | −6.95E−02 | 3.75E+00 | 4.35E−03 |
| ENSG00000090376 | IRAK3 | 5.47E+00 | 2.57E+00 | 5.63E+00 | 4.90E−04 |
| ENSG00000229092 | <NA> | 5.46E+00 | −8.67E−01 | 4.01E+00 | 3.10E−03 |
| ENSG00000248105 | <NA> | 5.46E+00 | −7.75E−02 | 3.77E+00 | 4.21E−03 |
| ENSG00000128040 | SPINK2 | 5.44E+00 | −7.88E−02 | 4.12E+00 | 2.71E−03 |
| ENSG00000010671 | BTK | 5.44E+00 | −8.11E−02 | 4.11E+00 | 2.73E−03 |
| ENSG00000211961 | <NA> | 5.43E+00 | −7.40E−02 | 3.93E+00 | 3.44E−03 |
| ENSG00000164627 | KIF6 | 5.39E+00 | 6.70E−01 | 3.65E+00 | 4.92E−03 |
| ENSG00000279404 | <NA> | 5.38E+00 | −9.07E−01 | 3.93E+00 | 3.41E−03 |
| ENSG00000253209 | <NA> | 5.38E+00 | −8.73E−01 | 3.39E+00 | 7.01E−03 |
| ENSG00000008441 | NFIX | 5.38E+00 | −1.42E−01 | 3.14E+00 | 9.84E−03 |
| ENSG00000170500 | LONRF2 | 5.37E+00 | 1.23E+00 | 2.92E+00 | 1.35E−02 |
| ENSG00000111321 | LTBR | 5.36E+00 | −9.01E−01 | 3.89E+00 | 3.61E−03 |
| ENSG00000123405 | NFE2 | 5.36E+00 | −8.83E−01 | 3.40E+00 | 6.86E−03 |
| ENSG00000174099 | MSRB3 | 5.34E+00 | 1.03E+00 | 4.20E+00 | 2.43E−03 |
| ENSG00000240411 | <NA> | 5.34E+00 | 2.29E−01 | 3.77E+00 | 4.19E−03 |
| ENSG00000124613 | ZNF391 | 5.32E+00 | −9.18E−01 | 3.78E+00 | 4.14E−03 |
| ENSG00000137486 | ARRB1 | 5.31E+00 | 2.45E+00 | 5.36E+00 | 6.41E−04 |
| ENSG00000129473 | BCL2L2 | 5.31E+00 | 1.35E+00 | 4.05E+00 | 2.91E−03 |
| ENSG00000122592 | HOXA7 | 5.30E+00 | −9.61E−01 | 3.58E+00 | 5.39E−03 |
| ENSG00000198300 | PEG3 | 5.30E+00 | −9.56E−01 | 3.75E+00 | 4.30E−03 |
| ENSG00000107104 | KANK1 | 5.30E+00 | 1.33E+00 | 3.31E+00 | 7.82E−03 |
| ENSG00000250241 | LOC101927359 | 5.29E+00 | 2.10E−01 | 3.46E+00 | 6.34E−03 |
| ENSG00000106004 | HOXA5 | 5.27E+00 | −1.00E+00 | 2.90E+00 | 1.38E−02 |
| ENSG00000198075 | SULT1C4 | 5.26E+00 | −9.81E−01 | 3.59E+00 | 5.33E−03 |
| ENSG00000248309 | MEF2C-AS1 | 5.25E+00 | −1.79E−01 | 3.86E+00 | 3.74E−03 |
| ENSG00000211694 | <NA> | 5.25E+00 | 1.60E+00 | 4.47E+00 | 1.75E−03 |
| ENSG00000283118 | <NA> | 5.25E+00 | −9.62E−01 | 3.83E+00 | 3.88E−03 |
| ENSG00000221351 | <NA> | 5.25E+00 | −9.69E−01 | 3.83E+00 | 3.86E−03 |
| ENSG00000123836 | PFKFB2 | 5.24E+00 | 4.68E+00 | 8.47E+00 | 4.08E−05 |
| ENSG00000073792 | IGF2BP2 | 5.20E+00 | 1.95E+00 | 4.78E+00 | 1.21E−03 |
| ENSG00000211821 | <NA> | 5.19E+00 | 5.57E−01 | 2.80E+00 | 1.61E−02 |
| ENSG00000174233 | ADCY6 | 5.18E+00 | −1.02E+00 | 3.38E+00 | 6.98E−03 |
| ENSG00000134873 | CLDN10 | 5.18E+00 | −9.68E−01 | 3.17E+00 | 9.38E−03 |
| ENSG00000175093 | SPSB4 | 5.18E+00 | −1.01E+00 | 3.74E+00 | 4.33E−03 |
| ENSG00000137177 | KIF13A | 5.15E+00 | −1.01E+00 | 3.72E+00 | 4.46E−03 |
| ENSG00000248803 | <NA> | 5.13E+00 | −1.04E+00 | 3.63E+00 | 4.96E−03 |
| ENSG00000161133 | USP41 | 5.12E+00 | −1.03E+00 | 3.69E+00 | 4.60E−03 |
| ENSG00000261251 | <NA> | 5.12E+00 | −1.03E+00 | 3.69E+00 | 4.60E−03 |
| ENSG00000276210 | LINC00226 | 5.10E+00 | −2.45E−01 | 3.77E+00 | 4.15E−03 |
| ENSG00000132746 | ALDH3B2 | 5.10E+00 | 1.41E+00 | 2.62E+00 | 2.08E−02 |
| ENSG00000211968 | <NA> | 5.09E+00 | −1.08E+00 | 3.00E+00 | 1.19E−02 |
| ENSG00000061337 | LZTS1 | 5.09E+00 | −2.38E−01 | 3.27E+00 | 8.07E−03 |
| ENSG00000262877 | <NA> | 5.08E+00 | −2.67E−01 | 3.67E+00 | 4.74E−03 |
| ENSG00000145362 | ANK2 | 5.08E+00 | −1.03E+00 | 3.35E+00 | 7.32E−03 |
| ENSG00000100321 | SYNGR1 | 5.07E+00 | 5.45E−01 | 3.45E+00 | 6.30E−03 |
| ENSG00000039560 | RAI14 | 5.06E+00 | −1.02E+00 | 2.69E+00 | 1.87E−02 |
| ENSG00000122025 | FLT3 | 5.04E+00 | 1.46E+00 | 3.90E+00 | 3.51E−03 |
| ENSG00000174348 | PODN | 5.03E+00 | −1.11E+00 | 3.17E+00 | 9.31E−03 |
| ENSG00000260188 | <NA> | 5.02E+00 | 7.05E−01 | 2.94E+00 | 1.30E−02 |
| ENSG00000172828 | CES3 | 5.01E+00 | −1.08E+00 | 3.57E+00 | 5.35E−03 |
| ENSG00000130649 | CYP2E1 | 4.99E+00 | 4.70E−01 | 3.28E+00 | 8.01E−03 |
| ENSG00000150681 | RGS18 | 4.98E+00 | 4.47E−02 | 3.01E+00 | 1.17E−02 |
| ENSG00000019485 | PRDM11 | 4.98E+00 | 7.71E−01 | 2.86E+00 | 1.46E−02 |
| ENSG00000276890 | <NA> | 4.96E+00 | −1.13E+00 | 3.30E+00 | 7.79E−03 |
| ENSG00000228203 | RNF144A-AS1 | 4.95E+00 | 2.49E−01 | 2.33E+00 | 3.21E−02 |
| ENSG00000104059 | FAM189A1 | 4.94E+00 | −1.09E+00 | 3.06E+00 | 1.09E−02 |
| ENSG00000127507 | ADGRE2 | 4.94E+00 | 4.09E+00 | 7.91E+00 | 6.16E−05 |
| ENSG00000119411 | BSPRY | 4.94E+00 | 2.85E−01 | 3.44E+00 | 6.40E−03 |
| ENSG00000104903 | LYL1 | 4.93E+00 | −1.12E+00 | 3.49E+00 | 5.92E−03 |
| ENSG00000266794 | <NA> | 4.93E+00 | −1.12E+00 | 3.47E+00 | 6.16E−03 |
| ENSG00000147459 | DOCK5 | 4.91E+00 | 4.63E+00 | 8.45E+00 | 4.08E−05 |
| ENSG00000062370 | ZNF112 | 4.91E+00 | −3.47E−01 | 3.57E+00 | 5.37E−03 |
| ENSG00000274482 | <NA> | 4.91E+00 | 3.08E+00 | 2.33E+00 | 3.18E−02 |
| ENSG00000248735 | <NA> | 4.90E+00 | 1.17E+00 | 3.19E+00 | 9.05E−03 |
| ENSG00000282639 | <NA> | 4.89E+00 | −3.50E−01 | 3.27E+00 | 8.03E−03 |
| ENSG00000168671 | UGT3A2 | 4.88E+00 | 3.52E+00 | 4.81E+00 | 1.15E−03 |
| ENSG00000114315 | HES1 | 4.88E+00 | 7.25E−01 | 2.73E+00 | 1.74E−02 |
| ENSG00000188280 | FAM230G | 4.87E+00 | −1.18E+00 | 3.19E+00 | 9.05E−03 |
| ENSG00000100365 | NCF4 | 4.85E+00 | 1.23E+00 | 2.48E+00 | 2.55E−02 |
| ENSG00000275063 | LOC102723407 | 4.85E+00 | 7.85E−01 | 3.77E+00 | 4.10E−03 |
| ENSG00000145936 | KCNMB1 | 4.85E+00 | −1.17E+00 | 3.41E+00 | 6.59E−03 |
| ENSG00000255642 | <NA> | 4.85E+00 | −1.17E+00 | 3.41E+00 | 6.59E−03 |
| ENSG00000119535 | CSF3R | 4.85E+00 | 4.36E−01 | 3.21E+00 | 8.73E−03 |
| ENSG00000273894 | <NA> | 4.84E+00 | −3.75E−01 | 3.24E+00 | 8.38E−03 |
| ENSG00000281392 | LINC00506 | 4.84E+00 | 1.87E+00 | 2.43E+00 | 2.74E−02 |
| ENSG00000146094 | DOK3 | 4.84E+00 | 7.87E−01 | 3.73E+00 | 4.33E−03 |
| ENSG00000123843 | C4BPB | 4.83E+00 | −1.22E+00 | 2.77E+00 | 1.65E−02 |
| ENSG00000174776 | WDR49 | 4.83E+00 | 1.03E+00 | 3.76E+00 | 4.15E−03 |
| ENSG00000087253 | LPCAT2 | 4.81E+00 | 1.09E+00 | 2.38E+00 | 2.94E−02 |
| ENSG00000233968 | LOC101928834 | 4.80E+00 | −4.64E−02 | 2.88E+00 | 1.39E−02 |
| ENSG00000164929 | BAALC | 4.78E+00 | −3.97E−01 | 3.38E+00 | 6.84E−03 |
| ENSG00000128309 | MPST | 4.78E+00 | 3.98E−01 | 3.12E+00 | 9.89E−03 |
| ENSG00000211962 | <NA> | 4.78E+00 | 9.21E−01 | 3.12E+00 | 9.84E−03 |
| ENSG00000165259 | HDX | 4.77E+00 | −1.22E+00 | 3.25E+00 | 8.26E−03 |
| ENSG00000153253 | SCN3A | 4.77E+00 | 5.02E+00 | 3.46E+00 | 6.14E−03 |
| ENSG00000214796 | <NA> | 4.77E+00 | −1.19E+00 | 3.19E+00 | 8.93E−03 |
| ENSG00000273415 | <NA> | 4.77E+00 | 9.90E−01 | 2.31E+00 | 3.27E−02 |
| ENSG00000196503 | ARL9 | 4.74E+00 | −1.21E+00 | 3.18E+00 | 9.01E−03 |
| ENSG00000104154 | SLC30A4 | 4.74E+00 | 1.74E−01 | 3.22E+00 | 8.52E−03 |
| ENSG00000238057 | ZEB2-AS1 | 4.72E+00 | −1.23E+00 | 3.28E+00 | 7.84E−03 |
| ENSG00000010438 | PRSS3 | 4.71E+00 | −1.22E+00 | 3.23E+00 | 8.43E−03 |
| ENSG00000170558 | CDH2 | 4.70E+00 | 2.23E+00 | 4.59E+00 | 1.47E−03 |
| ENSG00000177103 | DSCAML1 | 4.70E+00 | −4.90E−01 | 2.52E+00 | 2.36E−02 |
| ENSG00000163915 | IGF2BP2-AS1 | 4.69E+00 | −4.52E−01 | 3.35E+00 | 7.17E−03 |
| ENSG00000274307 | <NA> | 4.68E+00 | −1.23E+00 | 3.10E+00 | 1.02E−02 |
| ENSG00000172830 | SSH3 | 4.67E+00 | −1.25E+00 | 3.20E+00 | 8.72E−03 |
| ENSG00000102024 | PLS3 | 4.67E+00 | 2.17E+00 | 4.16E+00 | 2.49E−03 |
| ENSG00000238275 | <NA> | 4.66E+00 | −1.22E+00 | 2.76E+00 | 1.65E−02 |
| ENSG00000107968 | MAP3K8 | 4.65E+00 | −4.75E−01 | 3.30E+00 | 7.61E−03 |
| ENSG00000274840 | <NA> | 4.65E+00 | 3.38E−01 | 2.87E+00 | 1.40E−02 |
| ENSG00000205978 | NYNRIN | 4.65E+00 | 9.46E−01 | 3.67E+00 | 4.64E−03 |
| ENSG00000281327 | LINC01338 | 4.65E+00 | 6.11E−01 | 2.96E+00 | 1.24E−02 |
| ENSG00000211949 | <NA> | 4.64E+00 | −9.91E−02 | 3.15E+00 | 9.37E−03 |
| ENSG00000258909 | <NA> | 4.63E+00 | −1.27E+00 | 3.19E+00 | 8.89E−03 |
| ENSG00000206557 | TRIM71 | 4.63E+00 | −1.27E+00 | 3.17E+00 | 9.09E−03 |
| ENSG00000143167 | GPA33 | 4.63E+00 | 1.44E+00 | 2.20E+00 | 3.85E−02 |
| ENSG00000150760 | DOCK1 | 4.62E+00 | 2.63E+00 | 4.95E+00 | 9.75E−04 |
| ENSG00000128510 | CPA4 | 4.62E+00 | 1.27E+00 | 3.13E+00 | 9.60E−03 |
| ENSG00000184113 | CLDN5 | 4.61E+00 | −4.91E−01 | 3.24E+00 | 8.22E−03 |
| ENSG00000166342 | NETO1 | 4.60E+00 | 1.66E+00 | 3.87E+00 | 3.55E−03 |
| ENSG00000282320 | <NA> | 4.59E+00 | −5.11E−01 | 2.98E+00 | 1.20E−02 |
| ENSG00000211966 | <NA> | 4.58E+00 | −1.30E+00 | 3.14E+00 | 9.54E−03 |
| ENSG00000130202 | NECTIN2 | 4.58E+00 | −1.33E+00 | 2.92E+00 | 1.29E−02 |
| ENSG00000124813 | RUNX2 | 4.58E+00 | 5.02E+00 | 6.54E+00 | 1.96E−04 |
| ENSG00000272953 | <NA> | 4.58E+00 | −1.32E+00 | 3.02E+00 | 1.13E−02 |
| ENSG00000279652 | <NA> | 4.57E+00 | −4.93E−01 | 2.76E+00 | 1.63E−02 |
| ENSG00000048052 | HDAC9 | 4.56E+00 | 2.30E+00 | 2.79E+00 | 1.57E−02 |
| ENSG00000135525 | MAP7 | 4.56E+00 | 2.62E+00 | 3.68E+00 | 4.55E−03 |
| ENSG00000152580 | IGSF10 | 4.56E+00 | −1.32E+00 | 3.07E+00 | 1.05E−02 |
| ENSG00000148143 | ZNF462 | 4.56E+00 | 3.26E+00 | 5.55E+00 | 5.08E−04 |
| ENSG00000204764 | RANBP17 | 4.55E+00 | −1.29E+00 | 2.90E+00 | 1.34E−02 |
| ENSG00000171115 | GIMAP8 | 4.54E+00 | 9.66E−01 | 3.28E+00 | 7.83E−03 |
| ENSG00000169554 | ZEB2 | 4.54E+00 | 3.92E+00 | 2.91E+00 | 1.31E−02 |
| ENSG00000231793 | <NA> | 4.53E+00 | −1.36E+00 | 2.80E+00 | 1.55E−02 |
| ENSG00000258172 | <NA> | 4.53E+00 | −1.32E+00 | 3.08E+00 | 1.02E−02 |
| ENSG00000234456 | MAGI2-AS3 | 4.53E+00 | 5.86E−01 | 3.06E+00 | 1.06E−02 |
| ENSG00000234432 | LOC100129484 | 4.53E+00 | −5.49E−01 | 3.06E+00 | 1.06E−02 |
| ENSG00000211958 | <NA> | 4.51E+00 | 1.18E+00 | 2.66E+00 | 1.88E−02 |
| ENSG00000137726 | FXYD6 | 4.50E+00 | −1.32E+00 | 2.97E+00 | 1.21E−02 |
| ENSG00000105967 | TFEC | 4.50E+00 | −5.18E−01 | 2.48E+00 | 2.48E−02 |
| ENSG00000231718 | LOC400800 | 4.48E+00 | 3.99E−01 | 2.93E+00 | 1.27E−02 |
| ENSG00000254112 | <NA> | 4.48E+00 | −1.32E+00 | 2.79E+00 | 1.56E−02 |
| ENSG00000270885 | RASL10B | 4.48E+00 | −1.33E+00 | 2.89E+00 | 1.35E−02 |
| ENSG00000237667 | LINC01115 | 4.47E+00 | −1.38E+00 | 2.89E+00 | 1.36E−02 |
| ENSG00000265579 | <NA> | 4.46E+00 | −1.31E+00 | 2.52E+00 | 2.34E−02 |
| ENSG00000213997 | <NA> | 4.46E+00 | −1.35E+00 | 2.95E+00 | 1.24E−02 |
| ENSG00000251301 | LINC02384 | 4.45E+00 | 3.02E−02 | 2.94E+00 | 1.26E−02 |
| ENSG00000228014 | <NA> | 4.45E+00 | −5.79E−01 | 3.09E+00 | 1.01E−02 |
| ENSG00000139117 | CPNE8 | 4.43E+00 | −1.78E−01 | 2.41E+00 | 2.74E−02 |
| ENSG00000267199 | <NA> | 4.43E+00 | 2.05E−01 | 2.57E+00 | 2.16E−02 |
| ENSG00000196358 | NTNG2 | 4.43E+00 | 1.43E+00 | 2.09E+00 | 4.46E−02 |
| ENSG00000213973 | ZNF99 | 4.42E+00 | −1.37E+00 | 2.96E+00 | 1.22E−02 |
| ENSG00000227885 | <NA> | 4.39E+00 | −2.68E−01 | 2.42E+00 | 2.70E−02 |
| ENSG00000165568 | AKR1E2 | 4.39E+00 | −2.88E−04 | 2.88E+00 | 1.35E−02 |
| ENSG00000166166 | TRMT61A | 4.39E+00 | 3.02E−02 | 2.60E+00 | 2.07E−02 |
| ENSG00000125384 | PTGER2 | 4.38E+00 | −3.80E−03 | 2.58E+00 | 2.11E−02 |
| ENSG00000151692 | RNF144A | 4.37E+00 | 4.11E+00 | 4.66E+00 | 1.35E−03 |
| ENSG00000107833 | NPM3 | 4.37E+00 | −1.41E+00 | 2.91E+00 | 1.31E−02 |
| ENSG00000166866 | MYO1A | 4.36E+00 | −1.40E+00 | 2.88E+00 | 1.35E−02 |
| ENSG00000236244 | <NA> | 4.36E+00 | −1.40E+00 | 2.88E+00 | 1.35E−02 |
| ENSG00000225028 | <NA> | 4.36E+00 | −1.39E+00 | 2.77E+00 | 1.60E−02 |
| ENSG00000164066 | INTU | 4.36E+00 | 1.61E+00 | 3.39E+00 | 6.66E−03 |
| ENSG00000255354 | <NA> | 4.36E+00 | −1.42E+00 | 2.87E+00 | 1.37E−02 |
| ENSG00000271857 | <NA> | 4.36E+00 | −1.42E+00 | 2.87E+00 | 1.37E−02 |
| ENSG00000248393 | <NA> | 4.35E+00 | 1.48E+00 | 3.20E+00 | 8.66E−03 |
| ENSG00000162641 | AKNAD1 | 4.34E+00 | −1.44E+00 | 2.74E+00 | 1.66E−02 |
| ENSG00000241597 | <NA> | 4.34E+00 | −6.58E−01 | 2.48E+00 | 2.44E−02 |
| ENSG00000188916 | INSYN2 | 4.33E+00 | −1.44E+00 | 2.81E+00 | 1.50E−02 |
| ENSG00000135046 | ANXA1 | 4.33E+00 | 4.49E+00 | 3.99E+00 | 3.01E−03 |
| ENSG00000198720 | ANKRD13B | 4.32E+00 | −2.71E−01 | 2.63E+00 | 1.94E−02 |
| ENSG00000162755 | KLHDC9 | 4.32E+00 | −1.41E+00 | 2.75E+00 | 1.63E−02 |
| ENSG00000253709 | <NA> | 4.32E+00 | −1.41E+00 | 2.75E+00 | 1.63E−02 |
| ENSG00000272525 | <NA> | 4.32E+00 | −1.41E+00 | 2.75E+00 | 1.63E−02 |
| ENSG00000249978 | <NA> | 4.32E+00 | −6.86E−01 | 2.27E+00 | 3.35E−02 |
| ENSG00000237819 | LOC101927497 | 4.30E+00 | 3.61E+00 | 6.30E+00 | 2.40E−04 |
| ENSG00000235438 | LOC105370105 | 4.30E+00 | −1.46E+00 | 2.72E+00 | 1.71E−02 |
| ENSG00000248557 | <NA> | 4.30E+00 | −1.46E+00 | 2.72E+00 | 1.71E−02 |
| ENSG00000132359 | RAP1GAP2 | 4.30E+00 | 3.20E+00 | 4.05E+00 | 2.79E−03 |
| ENSG00000169413 | RNASE6 | 4.29E+00 | 2.48E+00 | 3.47E+00 | 5.94E−03 |
| ENSG00000180758 | GPR157 | 4.29E+00 | −6.55E−01 | 2.94E+00 | 1.24E−02 |
| ENSG00000108405 | P2RX1 | 4.28E+00 | 2.27E+00 | 3.92E+00 | 3.26E−03 |
| ENSG00000225302 | <NA> | 4.27E+00 | −6.70E−01 | 2.68E+00 | 1.80E−02 |
| ENSG00000116396 | KCNC4 | 4.27E+00 | 3.08E+00 | 2.94E+00 | 1.24E−02 |
| ENSG00000197816 | CCDC180 | 4.25E+00 | −1.48E+00 | 2.69E+00 | 1.77E−02 |
| ENSG00000198795 | ZNF521 | 4.25E+00 | −1.48E+00 | 2.69E+00 | 1.77E−02 |
| ENSG00000108452 | <NA> | 4.25E+00 | −1.46E+00 | 2.78E+00 | 1.55E−02 |
| ENSG00000251322 | SHANK3 | 4.25E+00 | −1.47E+00 | 2.78E+00 | 1.57E−02 |
| ENSG00000232166 | <NA> | 4.24E+00 | −1.46E+00 | 2.76E+00 | 1.61E−02 |
| ENSG00000143545 | RAB13 | 4.24E+00 | 8.16E−01 | 2.97E+00 | 1.18E−02 |
| ENSG00000145391 | SETD7 | 4.22E+00 | 3.17E+00 | 5.08E+00 | 8.26E−04 |
| ENSG00000163093 | BBS5 | 4.22E+00 | −1.46E+00 | 2.71E+00 | 1.74E−02 |
| ENSG00000164237 | CMBL | 4.20E+00 | −9.47E−02 | 2.73E+00 | 1.68E−02 |
| ENSG00000253449 | <NA> | 4.19E+00 | −1.47E+00 | 2.63E+00 | 1.95E−02 |
| ENSG00000071967 | CYBRD1 | 4.19E+00 | 2.70E+00 | 2.69E+00 | 1.77E−02 |
| ENSG00000196758 | LOC401040 | 4.18E+00 | −1.50E+00 | 2.72E+00 | 1.70E−02 |
| ENSG00000257060 | LOC105370982 | 4.18E+00 | 4.08E−01 | 2.68E+00 | 1.80E−02 |
| ENSG00000167705 | RILP | 4.18E+00 | −6.84E−01 | 2.61E+00 | 1.98E−02 |
| ENSG00000233829 | <NA> | 4.18E+00 | −3.44E−01 | 2.78E+00 | 1.56E−02 |
| ENSG00000139132 | FGD4 | 4.17E+00 | −1.49E+00 | 2.67E+00 | 1.82E−02 |
| ENSG00000225742 | LINC02036 | 4.17E+00 | −1.49E+00 | 2.67E+00 | 1.82E−02 |
| ENSG00000267369 | <NA> | 4.17E+00 | −1.49E+00 | 2.67E+00 | 1.82E−02 |
| ENSG00000215386 | MIR99AHG | 4.17E+00 | −7.37E−01 | 2.63E+00 | 1.94E−02 |
| ENSG00000136213 | CHST12 | 4.16E+00 | 3.55E+00 | 5.00E+00 | 9.02E−04 |
| ENSG00000075618 | FSCN1 | 4.15E+00 | 1.42E+00 | 3.13E+00 | 9.41E−03 |
| ENSG00000170456 | DENND5B | 4.12E+00 | 1.47E+00 | 3.01E+00 | 1.10E−02 |
| ENSG00000245317 | LOC100996419 | 4.11E+00 | −1.54E+00 | 2.64E+00 | 1.91E−02 |
| ENSG00000261997 | <NA> | 4.11E+00 | −1.54E+00 | 2.64E+00 | 1.91E−02 |
| ENSG00000271605 | MILR1 | 4.11E+00 | −1.54E+00 | 2.64E+00 | 1.91E−02 |
| ENSG00000186603 | HPDL | 4.11E+00 | −7.07E−01 | 2.34E+00 | 2.98E−02 |
| ENSG00000153721 | CNKSR3 | 4.11E+00 | −1.53E+00 | 2.63E+00 | 1.94E−02 |
| ENSG00000250155 | LOC107986412 | 4.11E+00 | −7.70E−01 | 2.37E+00 | 2.85E−02 |
| ENSG00000197993 | KEL | 4.11E+00 | −1.51E+00 | 2.51E+00 | 2.32E−02 |
| ENSG00000041353 | RAB27B | 4.10E+00 | 3.83E−01 | 2.86E+00 | 1.37E−02 |
| ENSG00000143776 | CDC42BPA | 4.10E+00 | 2.07E−01 | 2.58E+00 | 2.07E−02 |
| ENSG00000213062 | <NA> | 4.10E+00 | −7.31E−01 | 2.61E+00 | 2.00E−02 |
| ENSG00000145287 | PLAC8 | 4.09E+00 | 3.61E+00 | 3.91E+00 | 3.31E−03 |
| ENSG00000269961 | <NA> | 4.09E+00 | −1.56E+00 | 2.59E+00 | 2.05E−02 |
| ENSG00000145217 | SLC26A1 | 4.09E+00 | −1.53E+00 | 2.57E+00 | 2.10E−02 |
| ENSG00000225200 | <NA> | 4.09E+00 | −1.53E+00 | 2.57E+00 | 2.10E−02 |
| ENSG00000132744 | ACY3 | 4.09E+00 | −7.13E−01 | 2.12E+00 | 4.19E−02 |
| ENSG00000267127 | <NA> | 4.09E+00 | −7.65E−01 | 2.70E+00 | 1.73E−02 |
| ENSG00000128833 | MYO5C | 4.08E+00 | 2.71E+00 | 4.08E+00 | 2.66E−03 |
| ENSG00000211957 | <NA> | 4.07E+00 | 9.63E−01 | 2.38E+00 | 2.80E−02 |
| ENSG00000140022 | STON2 | 4.07E+00 | −1.24E−01 | 2.31E+00 | 3.09E−02 |
| ENSG00000235823 | OLMALINC | 4.07E+00 | 1.33E+00 | 2.31E+00 | 3.11E−02 |
| ENSG00000215146 | LOC441666 | 4.06E+00 | −7.62E−01 | 2.68E+00 | 1.79E−02 |
| ENSG00000256589 | <NA> | 4.06E+00 | −1.54E+00 | 2.49E+00 | 2.38E−02 |
| ENSG00000222724 | <NA> | 4.05E+00 | −3.96E−01 | 2.63E+00 | 1.92E−02 |
| ENSG00000102230 | PCYT1B | 4.05E+00 | 9.64E−01 | 2.42E+00 | 2.65E−02 |
| ENSG00000183605 | SFXN4 | 4.05E+00 | 8.92E−01 | 2.39E+00 | 2.74E−02 |
| ENSG00000105383 | CD33 | 4.05E+00 | −1.56E+00 | 2.57E+00 | 2.10E−02 |
| ENSG00000173705 | SUSD5 | 4.05E+00 | −1.56E+00 | 2.57E+00 | 2.10E−02 |
| ENSG00000128815 | WDFY4 | 4.04E+00 | 3.10E+00 | 3.68E+00 | 4.42E−03 |
| ENSG00000243175 | <NA> | 4.04E+00 | −7.92E−01 | 2.64E+00 | 1.91E−02 |
| ENSG00000162377 | COA7 | 4.03E+00 | 1.02E+00 | 2.34E+00 | 2.99E−02 |
| ENSG00000124743 | KLHL31 | 4.03E+00 | −1.58E+00 | 2.54E+00 | 2.20E−02 |
| ENSG00000167123 | CERCAM | 4.03E+00 | −1.58E+00 | 2.54E+00 | 2.20E−02 |
| ENSG00000070614 | NDST1 | 4.03E+00 | 2.09E+00 | 3.62E+00 | 4.80E−03 |
| ENSG00000250400 | <NA> | 4.02E+00 | 3.28E+00 | 4.05E+00 | 2.75E−03 |
| ENSG00000103202 | NME4 | 4.02E+00 | 1.91E+00 | 3.62E+00 | 4.80E−03 |
| ENSG00000156140 | ADAMTS3 | 4.02E+00 | −7.85E−01 | 2.67E+00 | 1.81E−02 |
| ENSG00000089041 | P2RX7 | 4.02E+00 | 1.01E−01 | 2.11E+00 | 4.22E−02 |
| ENSG00000154914 | USP43 | 4.02E+00 | −7.83E−01 | 2.65E+00 | 1.86E−02 |
| ENSG00000236296 | GUSBP5 | 4.01E+00 | −7.98E−01 | 2.65E+00 | 1.87E−02 |
| ENSG00000185010 | F8 | 4.01E+00 | −7.72E−01 | 2.52E+00 | 2.25E−02 |
| ENSG00000227747 | <NA> | 4.01E+00 | 1.65E+00 | 3.05E+00 | 1.05E−02 |
| ENSG00000234072 | LOC105374363 | 4.00E+00 | 1.67E+00 | 2.12E+00 | 4.15E−02 |
| ENSG00000116711 | PLA2G4A | 4.00E+00 | −1.57E+00 | 2.46E+00 | 2.47E−02 |
| ENSG00000232640 | <NA> | 4.00E+00 | −1.57E+00 | 2.46E+00 | 2.47E−02 |
| ENSG00000177640 | CASC2 | 3.98E+00 | −4.59E−01 | 2.44E+00 | 2.53E−02 |
| ENSG00000114019 | AMOTL2 | 3.97E+00 | −1.60E+00 | 2.48E+00 | 2.39E−02 |
| ENSG00000204682 | CASC10 | 3.96E+00 | −1.61E+00 | 2.48E+00 | 2.38E−02 |
| ENSG00000230881 | <NA> | 3.96E+00 | −1.61E+00 | 2.48E+00 | 2.38E−02 |
| ENSG00000091262 | ABCC6 | 3.96E+00 | −1.41E−02 | 2.43E+00 | 2.57E−02 |
| ENSG00000230091 | TMEM254-AS1 | 3.96E+00 | −8.05E−01 | 2.37E+00 | 2.81E−02 |
| ENSG00000167034 | NKX3-1 | 3.95E+00 | −8.39E−01 | 2.54E+00 | 2.19E−02 |
| ENSG00000267771 | <NA> | 3.95E+00 | −8.39E−01 | 2.54E+00 | 2.19E−02 |
| ENSG00000224400 | <NA> | 3.94E+00 | −1.60E+00 | 2.42E+00 | 2.59E−02 |
| ENSG00000072422 | RHOBTB1 | 3.93E+00 | 1.65E+00 | 3.15E+00 | 8.99E−03 |
| ENSG00000258476 | <NA> | 3.93E+00 | −8.27E−01 | 2.57E+00 | 2.10E−02 |
| ENSG00000229619 | MBNL1-AS1 | 3.92E+00 | 4.88E+00 | 4.85E+00 | 1.05E−03 |
| ENSG00000158089 | GALNT14 | 3.92E+00 | −2.02E−01 | 2.01E+00 | 4.89E−02 |
| ENSG00000132846 | ZBED3 | 3.92E+00 | 3.08E+00 | 4.99E+00 | 9.02E−04 |
| ENSG00000038945 | MSR1 | 3.91E+00 | −4.64E−01 | 2.24E+00 | 3.44E−02 |
| ENSG00000173401 | GLIPR1L1 | 3.91E+00 | −8.56E−01 | 2.51E+00 | 2.27E−02 |
| ENSG00000172986 | GXYLT2 | 3.90E+00 | 5.04E+00 | 7.83E+00 | 6.34E−05 |
| ENSG00000260418 | <NA> | 3.90E+00 | −1.64E+00 | 2.42E+00 | 2.61E−02 |
| ENSG00000229056 | HECW2-AS1 | 3.89E+00 | 7.71E−01 | 2.60E+00 | 1.98E−02 |
| ENSG00000248125 | <NA> | 3.89E+00 | −1.65E+00 | 2.42E+00 | 2.62E−02 |
| ENSG00000267454 | ZNF582-AS1 | 3.89E+00 | −8.86E−01 | 2.16E+00 | 3.89E−02 |
| ENSG00000263089 | <NA> | 3.88E+00 | −8.11E−02 | 2.29E+00 | 3.19E−02 |
| ENSG00000047597 | XK | 3.88E+00 | −1.64E+00 | 2.38E+00 | 2.76E−02 |
| ENSG00000198816 | ZNF358 | 3.88E+00 | −1.64E+00 | 2.38E+00 | 2.76E−02 |
| ENSG00000223764 | LINC02593 | 3.88E+00 | −1.64E+00 | 2.38E+00 | 2.76E−02 |
| ENSG00000253552 | HOXA-AS2 | 3.88E+00 | −1.64E+00 | 2.38E+00 | 2.76E−02 |
| ENSG00000159674 | SPON2 | 3.88E+00 | 1.99E+00 | 3.38E+00 | 6.55E−03 |
| ENSG00000136383 | ALPK3 | 3.87E+00 | 1.24E+00 | 3.15E+00 | 9.00E−03 |
| ENSG00000042493 | CAPG | 3.87E+00 | 1.98E+00 | 3.65E+00 | 4.55E−03 |
| ENSG00000162722 | TRIM58 | 3.87E+00 | −9.04E−01 | 2.19E+00 | 3.70E−02 |
| ENSG00000278473 | <NA> | 3.86E+00 | 1.01E+00 | 2.30E+00 | 3.09E−02 |
| ENSG00000229153 | EPHA1-AS1 | 3.86E+00 | 1.04E−01 | 2.29E+00 | 3.18E−02 |
| ENSG00000135436 | FAM186B | 3.85E+00 | 4.43E−01 | 2.23E+00 | 3.48E−02 |
| ENSG00000162687 | KCNT2 | 3.84E+00 | −8.77E−01 | 2.30E+00 | 3.10E−02 |
| ENSG00000229589 | ACVR2B-AS1 | 3.84E+00 | −8.83E−01 | 2.48E+00 | 2.37E−02 |
| ENSG00000280003 | <NA> | 3.84E+00 | −8.83E−01 | 2.48E+00 | 2.37E−02 |
| ENSG00000257298 | <NA> | 3.83E+00 | −2.80E−01 | 2.38E+00 | 2.77E−02 |
| ENSG00000198478 | SH3BGRL2 | 3.82E+00 | −9.08E−01 | 2.36E+00 | 2.83E−02 |
| ENSG00000253883 | <NA> | 3.82E+00 | 6.02E−01 | 2.03E+00 | 4.69E−02 |
| ENSG00000238149 | <NA> | 3.81E+00 | −9.39E−02 | 2.31E+00 | 3.05E−02 |
| ENSG00000029153 | ARNTL2 | 3.80E+00 | 1.46E+00 | 3.18E+00 | 8.59E−03 |
| ENSG00000240666 | <NA> | 3.80E+00 | −5.73E−01 | 2.00E+00 | 4.94E−02 |
| ENSG00000079482 | OPHN1 | 3.79E+00 | −5.61E−01 | 2.19E+00 | 3.68E−02 |
| ENSG00000198046 | ZNF667 | 3.79E+00 | 2.84E+00 | 4.45E+00 | 1.67E−03 |
| ENSG00000227191 | <NA> | 3.79E+00 | 3.84E+00 | 3.99E+00 | 2.94E−03 |
| ENSG00000108176 | DNAJC12 | 3.79E+00 | −3.15E−01 | 2.20E+00 | 3.60E−02 |
| ENSG00000019991 | HGF | 3.78E+00 | 1.54E+00 | 3.23E+00 | 7.97E−03 |
| ENSG00000278989 | <NA> | 3.78E+00 | −1.40E−01 | 2.27E+00 | 3.24E−02 |
| ENSG00000197472 | ZNF695 | 3.78E+00 | −1.32E−01 | 2.21E+00 | 3.55E−02 |
| ENSG00000227470 | <NA> | 3.76E+00 | 3.18E−02 | 2.27E+00 | 3.26E−02 |
| ENSG00000134780 | DAGLA | 3.75E+00 | −9.43E−01 | 2.32E+00 | 3.00E−02 |
| ENSG00000237172 | B3GNT9 | 3.73E+00 | −3.28E−01 | 2.29E+00 | 3.13E−02 |
| ENSG00000120594 | PLXDC2 | 3.73E+00 | 9.84E−01 | 2.62E+00 | 1.93E−02 |
| ENSG00000124343 | XG | 3.73E+00 | 1.66E−01 | 2.23E+00 | 3.43E−02 |
| ENSG00000205464 | ATP6AP1L | 3.73E+00 | 5.00E+00 | 6.67E+00 | 1.68E−04 |
| ENSG00000164251 | F2RL1 | 3.73E+00 | −1.72E+00 | 2.25E+00 | 3.33E−02 |
| ENSG00000228598 | MACC1-AS1 | 3.73E+00 | −1.72E+00 | 2.25E+00 | 3.33E−02 |
| ENSG00000231445 | <NA> | 3.73E+00 | −1.72E+00 | 2.25E+00 | 3.33E−02 |
| ENSG00000254046 | <NA> | 3.72E+00 | 2.79E−01 | 2.21E+00 | 3.52E−02 |
| ENSG00000114737 | CISH | 3.72E+00 | 2.76E+00 | 3.42E+00 | 6.13E−03 |
| ENSG00000137124 | ALDH1B1 | 3.72E+00 | −5.80E−01 | 2.09E+00 | 4.27E−02 |
| ENSG00000226306 | NPY6R | 3.72E+00 | −3.26E−01 | 2.29E+00 | 3.14E−02 |
| ENSG00000268658 | LINC00664 | 3.71E+00 | 1.94E−01 | 2.47E+00 | 2.37E−02 |
| ENSG00000138378 | STAT4 | 3.71E+00 | 3.24E+00 | 3.74E+00 | 4.02E−03 |
| ENSG00000281657 | <NA> | 3.71E+00 | 3.18E+00 | 3.46E+00 | 5.87E−03 |
| ENSG00000116649 | SRM | 3.71E+00 | 3.11E+00 | 3.23E+00 | 8.00E−03 |
| ENSG00000211973 | <NA> | 3.70E+00 | −5.55E−01 | 2.17E+00 | 3.75E−02 |
| ENSG00000110237 | ARHGEF17 | 3.69E+00 | 3.98E−01 | 2.43E+00 | 2.53E−02 |
| ENSG00000170153 | RNF150 | 3.68E+00 | 3.44E+00 | 4.84E+00 | 1.06E−03 |
| ENSG00000272986 | <NA> | 3.68E+00 | −5.97E−01 | 2.28E+00 | 3.15E−02 |
| ENSG00000235244 | DANT2 | 3.68E+00 | 8.78E−01 | 2.40E+00 | 2.64E−02 |
| ENSG00000169957 | ZNF768 | 3.67E+00 | −3.42E−01 | 1.99E+00 | 4.94E−02 |
| ENSG00000233306 | <NA> | 3.67E+00 | 4.50E−01 | 2.07E+00 | 4.36E−02 |
| ENSG00000225234 | <NA> | 3.67E+00 | −3.43E−01 | 2.21E+00 | 3.55E−02 |
| ENSG00000227268 | KLLN | 3.67E+00 | 1.23E+00 | 2.77E+00 | 1.52E−02 |
| ENSG00000116353 | MECR | 3.66E+00 | 1.43E+00 | 2.99E+00 | 1.12E−02 |
| ENSG00000239704 | CDRT4 | 3.66E+00 | −2.99E−02 | 2.07E+00 | 4.40E−02 |
| ENSG00000140932 | CMTM2 | 3.65E+00 | −2.82E−02 | 2.13E+00 | 3.99E−02 |
| ENSG00000146373 | RNF217 | 3.65E+00 | −9.46E−01 | 2.00E+00 | 4.86E−02 |
| ENSG00000146858 | ZC3HAV1L | 3.65E+00 | 2.85E+00 | 3.54E+00 | 5.21E−03 |
| ENSG00000188404 | SELL | 3.64E+00 | 6.25E+00 | 4.61E+00 | 1.38E−03 |
| ENSG00000251248 | <NA> | 3.63E+00 | −9.77E−01 | 2.27E+00 | 3.22E−02 |
| ENSG00000275805 | <NA> | 3.62E+00 | −6.40E−01 | 2.13E+00 | 3.95E−02 |
| ENSG00000021826 | CPS1 | 3.62E+00 | 6.25E−01 | 2.61E+00 | 1.93E−02 |
| ENSG00000167291 | TBC1D16 | 3.62E+00 | 1.41E+00 | 2.13E+00 | 3.94E−02 |
| ENSG00000110492 | MDK | 3.61E+00 | 1.31E+00 | 2.63E+00 | 1.88E−02 |
| ENSG00000164116 | GUCY1A1 | 3.61E+00 | 4.31E+00 | 5.73E+00 | 4.09E−04 |
| ENSG00000105472 | CLEC11A | 3.61E+00 | 6.83E−01 | 2.05E+00 | 4.49E−02 |
| ENSG00000276231 | PIK3R6 | 3.61E+00 | 2.67E+00 | 2.64E+00 | 1.84E−02 |
| ENSG00000175857 | GAPT | 3.59E+00 | 1.64E+00 | 2.06E+00 | 4.44E−02 |
| ENSG00000196976 | LAGE3 | 3.59E+00 | −3.59E−02 | 2.10E+00 | 4.15E−02 |
| ENSG00000177096 | PHETA2 | 3.59E+00 | −2.16E−01 | 2.13E+00 | 3.97E−02 |
| ENSG00000254602 | <NA> | 3.59E+00 | −1.02E+00 | 2.17E+00 | 3.73E−02 |
| ENSG00000274561 | <NA> | 3.58E+00 | 1.57E+00 | 3.16E+00 | 8.80E−03 |
| ENSG00000200114 | <NA> | 3.57E+00 | −1.00E+00 | 2.04E+00 | 4.56E−02 |
| ENSG00000144045 | DQX1 | 3.56E+00 | 9.13E−02 | 2.03E+00 | 4.58E−02 |
| ENSG00000152611 | CAPSL | 3.56E+00 | 6.90E−02 | 2.06E+00 | 4.41E−02 |
| ENSG00000255084 | LOC101928896 | 3.55E+00 | −6.71E−01 | 2.10E+00 | 4.13E−02 |
| ENSG00000162654 | GBP4 | 3.55E+00 | 2.98E+00 | 2.37E+00 | 2.75E−02 |
| ENSG00000272701 | <NA> | 3.54E+00 | −1.04E+00 | 2.14E+00 | 3.90E−02 |
| ENSG00000166821 | PEX11A | 3.53E+00 | −4.26E−01 | 2.12E+00 | 4.03E−02 |
| ENSG00000279415 | <NA> | 3.53E+00 | −2.34E−01 | 2.23E+00 | 3.37E−02 |
| ENSG00000155974 | GRIP1 | 3.52E+00 | 4.55E−01 | 2.27E+00 | 3.19E−02 |
| ENSG00000224429 | <NA> | 3.51E+00 | 1.57E+00 | 2.35E+00 | 2.83E−02 |
| ENSG00000204740 | MALRD1 | 3.51E+00 | 1.59E+00 | 3.10E+00 | 9.49E−03 |
| ENSG00000280079 | <NA> | 3.50E+00 | 1.54E+00 | 3.00E+00 | 1.10E−02 |
| ENSG00000173548 | SNX33 | 3.49E+00 | −4.50E−01 | 2.07E+00 | 4.30E−02 |
| ENSG00000211938 | <NA> | 3.48E+00 | −4.36E−01 | 2.05E+00 | 4.46E−02 |
| ENSG00000226686 | LINC01535 | 3.48E+00 | −1.04E+00 | 2.07E+00 | 4.33E−02 |
| ENSG00000230295 | <NA> | 3.46E+00 | −1.07E+00 | 2.10E+00 | 4.11E−02 |
| ENSG00000268119 | LOC105372321 | 3.45E+00 | 2.48E+00 | 3.21E+00 | 8.05E−03 |
| ENSG00000211950 | <NA> | 3.45E+00 | 4.54E+00 | 5.97E+00 | 3.20E−04 |
| ENSG00000099256 | PRTFDC1 | 3.44E+00 | 1.92E+00 | 3.15E+00 | 8.84E−03 |
| ENSG00000262222 | <NA> | 3.43E+00 | −1.07E+00 | 2.04E+00 | 4.52E−02 |
| ENSG00000225172 | <NA> | 3.43E+00 | 1.77E+00 | 2.56E+00 | 2.06E−02 |
| ENSG00000137752 | CASP1 | 3.43E+00 | 1.31E+00 | 2.70E+00 | 1.68E−02 |
| ENSG00000076258 | FMO4 | 3.42E+00 | 6.89E−02 | 2.28E+00 | 3.10E−02 |
| ENSG00000140479 | PCSK6 | 3.41E+00 | −1.11E+00 | 2.00E+00 | 4.77E−02 |
| ENSG00000169313 | P2RY12 | 3.41E+00 | −1.11E+00 | 2.00E+00 | 4.77E−02 |
| ENSG00000073536 | NLE1 | 3.41E+00 | 2.11E+00 | 2.91E+00 | 1.23E−02 |
| ENSG00000198947 | DMD | 3.40E+00 | −4.93E−01 | 1.99E+00 | 4.84E−02 |
| ENSG00000251002 | <NA> | 3.39E+00 | 3.73E+00 | 3.07E+00 | 9.83E−03 |
| ENSG00000251669 | FAM86EP | 3.38E+00 | −1.12E+00 | 2.01E+00 | 4.66E−02 |
| ENSG00000148175 | STOM | 3.38E+00 | 1.48E+00 | 2.84E+00 | 1.37E−02 |
| ENSG00000186314 | PRELID2 | 3.37E+00 | 1.17E+00 | 2.62E+00 | 1.86E−02 |
| ENSG00000117586 | TNFSF4 | 3.36E+00 | 4.21E+00 | 4.48E+00 | 1.58E−03 |
| ENSG00000280399 | <NA> | 3.34E+00 | 2.34E+00 | 2.63E+00 | 1.84E−02 |
| ENSG00000140682 | TGFB1I1 | 3.34E+00 | −3.51E−01 | 2.02E+00 | 4.64E−02 |
| ENSG00000126016 | AMOT | 3.33E+00 | 2.20E+00 | 3.02E+00 | 1.05E−02 |
| ENSG00000227006 | <NA> | 3.31E+00 | 2.66E+00 | 2.01E+00 | 4.68E−02 |
| ENSG00000128487 | SPECC1 | 3.30E+00 | 3.12E+00 | 4.15E+00 | 2.37E−03 |
| ENSG00000221926 | TRIM16 | 3.30E+00 | 1.86E+00 | 2.60E+00 | 1.91E−02 |
| ENSG00000173905 | GOLIM4 | 3.29E+00 | 3.31E+00 | 3.41E+00 | 6.17E−03 |
| ENSG00000189337 | KAZN | 3.28E+00 | 4.51E−01 | 2.23E+00 | 3.31E−02 |
| ENSG00000125388 | GRK4 | 3.27E+00 | 1.08E+00 | 2.57E+00 | 2.01E−02 |
| ENSG00000250280 | <NA> | 3.27E+00 | −3.76E−01 | 2.01E+00 | 4.64E−02 |
| ENSG00000160360 | GPSM1 | 3.25E+00 | 1.00E+00 | 2.53E+00 | 2.11E−02 |
| ENSG00000152642 | GPD1L | 3.25E+00 | 2.63E+00 | 3.04E+00 | 1.02E−02 |
| ENSG00000267023 | <NA> | 3.24E+00 | 2.09E+00 | 2.21E+00 | 3.39E−02 |
| ENSG00000178538 | CA8 | 3.24E+00 | 3.21E+00 | 3.94E+00 | 3.06E−03 |
| ENSG00000211698 | <NA> | 3.24E+00 | 5.86E−01 | 2.22E+00 | 3.35E−02 |
| ENSG00000170275 | CRTAP | 3.23E+00 | 2.99E+00 | 4.04E+00 | 2.69E−03 |
| ENSG00000244405 | ETV5 | 3.22E+00 | 1.93E+00 | 2.13E+00 | 3.87E−02 |
| ENSG00000276449 | <NA> | 3.22E+00 | 1.01E+00 | 2.27E+00 | 3.12E−02 |
| ENSG00000112290 | WASF1 | 3.21E+00 | 3.56E+00 | 3.81E+00 | 3.61E−03 |
| ENSG00000128039 | SRD5A3 | 3.20E+00 | 1.66E+00 | 2.04E+00 | 4.39E−02 |
| ENSG00000197134 | ZNF257 | 3.19E+00 | 2.33E+00 | 2.33E+00 | 2.86E−02 |
| ENSG00000280832 | GSEC | 3.17E+00 | 2.18E+00 | 3.06E+00 | 9.86E−03 |
| ENSG00000180592 | SKIDA1 | 3.16E+00 | −4.99E−02 | 2.01E+00 | 4.61E−02 |
| ENSG00000108861 | DUSP3 | 3.16E+00 | 6.88E−01 | 2.06E+00 | 4.30E−02 |
| ENSG00000188171 | ZNF626 | 3.14E+00 | 2.06E+00 | 2.43E+00 | 2.44E−02 |
| ENSG00000138650 | PCDH10 | 3.14E+00 | 5.03E+00 | 5.13E+00 | 7.57E−04 |
| ENSG00000159399 | HK2 | 3.14E+00 | 4.08E+00 | 3.06E+00 | 9.78E−03 |
| ENSG00000184014 | DENND5A | 3.13E+00 | 5.08E+00 | 3.90E+00 | 3.23E−03 |
| ENSG00000254535 | PABPC4L | 3.13E+00 | 2.76E+00 | 3.13E+00 | 8.91E−03 |
| ENSG00000237854 | <NA> | 3.13E+00 | 1.64E+00 | 2.77E+00 | 1.49E−02 |
| ENSG00000183735 | TBK1 | 3.11E+00 | 1.40E+00 | 2.31E+00 | 2.90E−02 |
| ENSG00000160062 | ZBTB8A | 3.10E+00 | 1.43E+00 | 2.61E+00 | 1.86E−02 |
| ENSG00000270550 | <NA> | 3.09E+00 | −9.06E−02 | 1.95E+00 | 4.99E−02 |
| ENSG00000106605 | BLVRA | 3.09E+00 | 7.64E−01 | 2.11E+00 | 3.93E−02 |
| ENSG00000034053 | APBA2 | 3.08E+00 | 4.11E+00 | 2.43E+00 | 2.44E−02 |
| ENSG00000213468 | FIRRE | 3.08E+00 | 4.11E+00 | 2.46E+00 | 2.34E−02 |
| ENSG00000153707 | PTPRD | 3.07E+00 | 5.36E+00 | 5.26E+00 | 6.60E−04 |
| ENSG00000133460 | SLC2A11 | 3.07E+00 | 1.61E+00 | 2.42E+00 | 2.46E−02 |
| ENSG00000126947 | ARMCX1 | 3.07E+00 | 1.91E+00 | 2.19E+00 | 3.46E−02 |
| ENSG00000213949 | ITGA1 | 3.07E+00 | 2.42E+00 | 3.06E+00 | 9.75E−03 |
| ENSG00000085871 | MGST2 | 3.05E+00 | 1.53E+00 | 2.26E+00 | 3.11E−02 |
| ENSG00000107815 | TWNK | 3.04E+00 | 2.06E+00 | 3.00E+00 | 1.07E−02 |
| ENSG00000211829 | <NA> | 3.03E+00 | 4.48E+00 | 3.90E+00 | 3.19E−03 |
| ENSG00000134940 | ACRV1 | 3.03E+00 | 1.26E+00 | 2.35E+00 | 2.72E−02 |
| ENSG00000272512 | <NA> | 3.00E+00 | 9.39E−01 | 1.98E+00 | 4.77E−02 |
| ENSG00000234912 | SNHG20 | 2.99E+00 | 1.14E+00 | 2.17E+00 | 3.58E−02 |
| ENSG00000198298 | ZNF485 | 2.99E+00 | 8.08E−01 | 2.18E+00 | 3.49E−02 |
| ENSG00000134504 | KCTD1 | 2.99E+00 | 1.61E+00 | 2.45E+00 | 2.34E−02 |
| ENSG00000177398 | UMODL1 | 2.98E+00 | 2.78E+00 | 2.85E+00 | 1.31E−02 |
| ENSG00000128394 | APOBEC3F | 2.97E+00 | 2.33E+00 | 2.91E+00 | 1.20E−02 |
| ENSG00000153404 | PLEKHG4B | 2.97E+00 | 2.71E+00 | 3.17E+00 | 8.40E−03 |
| ENSG00000139926 | FRMD6 | 2.96E+00 | 3.95E+00 | 3.95E+00 | 2.98E−03 |
| ENSG00000182557 | SPNS3 | 2.96E+00 | 2.19E+00 | 2.80E+00 | 1.41E−02 |
| ENSG00000185304 | RGPD2 | 2.96E+00 | 2.62E+00 | 3.22E+00 | 7.82E−03 |
| ENSG00000114757 | PEX5L | 2.94E+00 | 3.84E+00 | 3.95E+00 | 3.00E−03 |
| ENSG00000103550 | KNOP1 | 2.93E+00 | 1.45E+00 | 2.13E+00 | 3.79E−02 |
| ENSG00000234345 | <NA> | 2.91E+00 | 1.12E+00 | 2.18E+00 | 3.51E−02 |
| ENSG00000112218 | GPR63 | 2.91E+00 | 1.13E+00 | 2.21E+00 | 3.33E−02 |
| ENSG00000217527 | <NA> | 2.91E+00 | 1.21E+00 | 2.11E+00 | 3.89E−02 |
| ENSG00000152990 | ADGRA3 | 2.91E+00 | 4.10E+00 | 3.67E+00 | 4.26E−03 |
| ENSG00000204287 | HLA-DRA | 2.90E+00 | 3.68E+00 | 1.96E+00 | 4.87E−02 |
| ENSG00000196549 | MME | 2.90E+00 | 6.02E+00 | 3.29E+00 | 7.06E−03 |
| ENSG00000099194 | SCD | 2.89E+00 | 5.69E+00 | 2.67E+00 | 1.71E−02 |
| ENSG00000100350 | FOXRED2 | 2.88E+00 | 3.00E+00 | 3.31E+00 | 6.90E−03 |
| ENSG00000275209 | <NA> | 2.88E+00 | 4.79E+00 | 3.82E+00 | 3.53E−03 |
| ENSG00000235492 | LINC01221 | 2.88E+00 | 3.10E+00 | 2.72E+00 | 1.57E−02 |
| ENSG00000278090 | <NA> | 2.88E+00 | 3.04E+00 | 2.28E+00 | 3.01E−02 |
| ENSG00000106484 | MEST | 2.87E+00 | 3.00E+00 | 2.78E+00 | 1.44E−02 |
| ENSG00000152207 | CYSLTR2 | 2.87E+00 | 1.56E+00 | 2.39E+00 | 2.54E−02 |
| ENSG00000105737 | GRIK5 | 2.87E+00 | 1.56E+00 | 2.44E+00 | 2.37E−02 |
| ENSG00000206190 | ATP10A | 2.86E+00 | 2.81E+00 | 3.08E+00 | 9.46E−03 |
| ENSG00000171132 | PRKCE | 2.85E+00 | 3.72E+00 | 2.72E+00 | 1.57E−02 |
| ENSG00000060138 | YBX3 | 2.85E+00 | 5.94E+00 | 2.23E+00 | 3.23E−02 |
| ENSG00000174749 | FAM241A | 2.85E+00 | 3.45E+00 | 3.31E+00 | 6.90E−03 |
| ENSG00000144893 | MED12L | 2.84E+00 | 4.01E+00 | 4.18E+00 | 2.24E−03 |
| ENSG00000266074 | BAHCC1 | 2.84E+00 | 6.06E+00 | 4.48E+00 | 1.56E−03 |
| ENSG00000101082 | SLA2 | 2.83E+00 | 3.63E+00 | 2.87E+00 | 1.26E−02 |
| ENSG00000138722 | MMRN1 | 2.83E+00 | 1.10E+00 | 1.99E+00 | 4.65E−02 |
| ENSG00000164932 | CTHRC1 | 2.83E+00 | 2.24E+00 | 2.87E+00 | 1.27E−02 |
| ENSG00000181631 | P2RY13 | 2.82E+00 | 1.34E+00 | 2.03E+00 | 4.38E−02 |
| ENSG00000254087 | LYN | 2.82E+00 | 1.45E+00 | 2.25E+00 | 3.14E−02 |
| ENSG00000141447 | OSBPL1A | 2.82E+00 | 3.37E+00 | 3.20E+00 | 8.02E−03 |
| ENSG00000267886 | ZNF730 | 2.81E+00 | 2.43E+00 | 2.90E+00 | 1.22E−02 |
| ENSG00000227403 | LINC01806 | 2.80E+00 | 2.09E+00 | 2.63E+00 | 1.79E−02 |
| ENSG00000179583 | CIITA | 2.80E+00 | 4.18E+00 | 4.43E+00 | 1.67E−03 |
| ENSG00000143179 | UCK2 | 2.78E+00 | 4.78E+00 | 3.09E+00 | 9.37E−03 |
| ENSG00000181481 | RNF135 | 2.78E+00 | 7.66E−01 | 1.96E+00 | 4.84E−02 |
| ENSG00000145220 | LYAR | 2.78E+00 | 2.36E+00 | 2.73E+00 | 1.56E−02 |
| ENSG00000111696 | NT5DC3 | 2.77E+00 | 1.16E+00 | 2.13E+00 | 3.72E−02 |
| ENSG00000168899 | VAMP5 | 2.76E+00 | 1.60E+00 | 1.98E+00 | 4.68E−02 |
| ENSG00000226950 | DANCR | 2.76E+00 | 3.74E+00 | 3.52E+00 | 5.18E−03 |
| ENSG00000136161 | RCBTB2 | 2.76E+00 | 3.94E+00 | 3.28E+00 | 7.12E−03 |
| ENSG00000228223 | HCG11 | 2.76E+00 | 2.22E+00 | 2.32E+00 | 2.83E−02 |
| ENSG00000109576 | AADAT | 2.75E+00 | 2.87E+00 | 2.90E+00 | 1.22E−02 |
| ENSG00000037241 | RPL26L1 | 2.75E+00 | 1.76E+00 | 2.33E+00 | 2.77E−02 |
| ENSG00000112195 | TREML2 | 2.75E+00 | 3.27E+00 | 3.40E+00 | 6.06E−03 |
| ENSG00000128602 | SMO | 2.74E+00 | 1.65E+00 | 2.30E+00 | 2.89E−02 |
| ENSG00000279620 | <NA> | 2.73E+00 | 1.40E+00 | 2.07E+00 | 4.07E−02 |
| ENSG00000260855 | <NA> | 2.73E+00 | 2.41E+00 | 2.10E+00 | 3.92E−02 |
| ENSG00000163297 | ANTXR2 | 2.73E+00 | 3.93E+00 | 2.49E+00 | 2.17E−02 |
| ENSG00000247081 | BAALC-AS1 | 2.73E+00 | 1.48E+00 | 2.11E+00 | 3.84E−02 |
| ENSG00000253325 | <NA> | 2.73E+00 | 1.25E+00 | 1.94E+00 | 4.96E−02 |
| ENSG00000095932 | SMIM24 | 2.72E+00 | 3.77E+00 | 3.36E+00 | 6.42E−03 |
| ENSG00000117016 | RIMS3 | 2.71E+00 | 3.96E+00 | 3.74E+00 | 3.90E−03 |
| ENSG00000164292 | RHOBTB3 | 2.71E+00 | 3.91E+00 | 3.97E+00 | 2.92E−03 |
| ENSG00000073060 | SCARB1 | 2.70E+00 | 2.24E+00 | 2.44E+00 | 2.37E−02 |
| ENSG00000134897 | BIVM | 2.69E+00 | 4.11E+00 | 3.18E+00 | 8.24E−03 |
| ENSG00000007237 | GAS7 | 2.68E+00 | 5.25E+00 | 4.80E+00 | 1.08E−03 |
| ENSG00000146530 | VWDE | 2.66E+00 | 2.38E+00 | 1.94E+00 | 4.95E−02 |
| ENSG00000106560 | GIMAP2 | 2.66E+00 | 4.48E+00 | 3.45E+00 | 5.66E−03 |
| ENSG00000044459 | CNTLN | 2.63E+00 | 4.15E+00 | 3.59E+00 | 4.74E−03 |
| ENSG00000113658 | SMAD5 | 2.63E+00 | 3.74E+00 | 2.64E+00 | 1.75E−02 |
| ENSG00000151376 | ME3 | 2.62E+00 | 2.41E+00 | 2.36E+00 | 2.64E−02 |
| ENSG00000086730 | LAT2 | 2.60E+00 | 3.01E+00 | 2.56E+00 | 1.97E−02 |
| ENSG00000101911 | PRPS2 | 2.59E+00 | 5.71E+00 | 4.87E+00 | 9.90E−04 |
| ENSG00000130635 | COL5A1 | 2.58E+00 | 1.84E+00 | 2.02E+00 | 4.34E−02 |
| ENSG00000179958 | DCTPP1 | 2.58E+00 | 2.26E+00 | 2.24E+00 | 3.13E−02 |
| ENSG00000100522 | GNPNAT1 | 2.57E+00 | 1.53E+00 | 1.98E+00 | 4.64E−02 |
| ENSG00000166825 | ANPEP | 2.56E+00 | 2.07E+00 | 2.07E+00 | 4.06E−02 |
| ENSG00000121858 | TNFSF10 | 2.54E+00 | 1.94E+00 | 1.96E+00 | 4.79E−02 |
| ENSG00000169126 | ARMC4 | 2.51E+00 | 1.76E+00 | 2.03E+00 | 4.30E−02 |
| ENSG00000170017 | ALCAM | 2.51E+00 | 2.62E+00 | 1.99E+00 | 4.52E−02 |
| ENSG00000175711 | B3GNTL1 | 2.51E+00 | 2.80E+00 | 2.66E+00 | 1.68E−02 |
| ENSG00000080493 | SLC4A4 | 2.51E+00 | 3.65E+00 | 3.41E+00 | 6.00E−03 |
| ENSG00000232677 | LINC00665 | 2.51E+00 | 3.82E+00 | 3.48E+00 | 5.45E−03 |
| ENSG00000153165 | RGPD3 | 2.50E+00 | 1.85E+00 | 1.94E+00 | 4.91E−02 |
| ENSG00000060982 | BCAT1 | 2.49E+00 | 5.09E+00 | 3.70E+00 | 4.07E−03 |
| ENSG00000183077 | AFMID | 2.49E+00 | 2.07E+00 | 2.23E+00 | 3.19E−02 |
| ENSG00000138449 | SLC40A1 | 2.49E+00 | 2.92E+00 | 2.65E+00 | 1.73E−02 |
| ENSG00000279496 | <NA> | 2.47E+00 | 2.66E+00 | 2.22E+00 | 3.20E−02 |
| ENSG00000105270 | CLIP3 | 2.46E+00 | 4.39E+00 | 2.59E+00 | 1.87E−02 |
| ENSG00000275004 | ZNF280B | 2.44E+00 | 2.79E+00 | 2.53E+00 | 2.03E−02 |
| ENSG00000117322 | CR2 | 2.44E+00 | 5.29E+00 | 3.37E+00 | 6.28E−03 |
| ENSG00000105810 | CDK6 | 2.40E+00 | 9.77E+00 | 5.50E+00 | 5.04E−04 |
| ENSG00000061918 | GUCY1B1 | 2.39E+00 | 4.17E+00 | 3.33E+00 | 6.59E−03 |
| ENSG00000106772 | PRUNE2 | 2.38E+00 | 3.47E+00 | 2.16E+00 | 3.50E−02 |
| ENSG00000109458 | GAB1 | 2.38E+00 | 4.32E+00 | 3.52E+00 | 5.17E−03 |
| ENSG00000168904 | LRRC28 | 2.37E+00 | 5.98E+00 | 3.39E+00 | 6.09E−03 |
| ENSG00000180385 | <NA> | 2.37E+00 | 3.92E+00 | 2.85E+00 | 1.29E−02 |
| ENSG00000171867 | PRNP | 2.36E+00 | 2.69E+00 | 2.12E+00 | 3.71E−02 |
| ENSG00000136997 | MYC | 2.35E+00 | 5.63E+00 | 2.12E+00 | 3.73E−02 |
| ENSG00000124217 | MOCS3 | 2.35E+00 | 2.41E+00 | 2.23E+00 | 3.15E−02 |
| ENSG00000282851 | BISPR | 2.35E+00 | 2.24E+00 | 1.94E+00 | 4.87E−02 |
| ENSG00000076248 | UNG | 2.35E+00 | 3.75E+00 | 2.55E+00 | 1.97E−02 |
| ENSG00000260260 | SNHG19 | 2.32E+00 | 2.94E+00 | 2.08E+00 | 3.96E−02 |
| ENSG00000188227 | ZNF793 | 2.32E+00 | 2.78E+00 | 2.09E+00 | 3.87E−02 |
| ENSG00000021355 | SERPINB1 | 2.31E+00 | 4.53E+00 | 2.89E+00 | 1.21E−02 |
| ENSG00000205336 | ADGRG1 | 2.30E+00 | 5.94E+00 | 3.81E+00 | 3.53E−03 |
| ENSG00000189283 | FHIT | 2.30E+00 | 4.32E+00 | 2.06E+00 | 4.05E−02 |
| ENSG00000185252 | ZNF74 | 2.29E+00 | 1.95E+00 | 1.92E+00 | 4.97E−02 |
| ENSG00000158220 | ESYT3 | 2.28E+00 | 4.30E+00 | 2.52E+00 | 2.07E−02 |
| ENSG00000133142 | TCEAL4 | 2.27E+00 | 4.16E+00 | 3.18E+00 | 8.07E−03 |
| ENSG00000149485 | FADS1 | 2.26E+00 | 4.99E+00 | 2.59E+00 | 1.85E−02 |
| ENSG00000182095 | TNRC18 | 2.26E+00 | 4.82E+00 | 3.56E+00 | 4.90E−03 |
| ENSG00000172172 | MRPL13 | 2.25E+00 | 2.59E+00 | 1.95E+00 | 4.75E−02 |
| ENSG00000143479 | DYRK3 | 2.25E+00 | 3.23E+00 | 2.42E+00 | 2.38E−02 |
| ENSG00000143162 | CREG1 | 2.25E+00 | 2.28E+00 | 2.02E+00 | 4.27E−02 |
| ENSG00000165028 | NIPSNAP3B | 2.24E+00 | 2.13E+00 | 1.97E+00 | 4.63E−02 |
| ENSG00000109787 | KLF3 | 2.23E+00 | 4.00E+00 | 2.06E+00 | 4.05E−02 |
| ENSG00000278730 | <NA> | 2.23E+00 | 4.08E+00 | 2.09E+00 | 3.87E−02 |
| ENSG00000132382 | MYBBP1A | 2.23E+00 | 3.43E+00 | 1.98E+00 | 4.57E−02 |
| ENSG00000157557 | ETS2 | 2.22E+00 | 5.40E+00 | 3.15E+00 | 8.40E−03 |
| ENSG00000065923 | SLC9A7 | 2.22E+00 | 4.07E+00 | 2.80E+00 | 1.39E−02 |
| ENSG00000132849 | PATJ | 2.20E+00 | 3.33E+00 | 2.58E+00 | 1.89E−02 |
| ENSG00000154447 | SH3RF1 | 2.20E+00 | 3.00E+00 | 2.16E+00 | 3.46E−02 |
| ENSG00000164930 | FZD6 | 2.20E+00 | 4.20E+00 | 2.56E+00 | 1.94E−02 |
| ENSG00000177425 | PAWR | 2.20E+00 | 3.93E+00 | 2.54E+00 | 1.99E−02 |
| ENSG00000163006 | CCDC138 | 2.19E+00 | 4.82E+00 | 2.97E+00 | 1.08E−02 |
| ENSG00000197965 | MPZL1 | 2.19E+00 | 5.10E+00 | 3.20E+00 | 7.94E−03 |
| ENSG00000197128 | ZNF772 | 2.18E+00 | 4.04E+00 | 2.38E+00 | 2.51E−02 |
| ENSG00000118640 | VAMP8 | 2.17E+00 | 3.57E+00 | 2.08E+00 | 3.90E−02 |
| ENSG00000226806 | LOC100507600 | 2.17E+00 | 6.35E+00 | 3.01E+00 | 1.02E−02 |
| ENSG00000138326 | RPS24 | 2.17E+00 | 6.50E+00 | 3.33E+00 | 6.61E−03 |
| ENSG00000161551 | ZNF577 | 2.17E+00 | 3.74E+00 | 2.62E+00 | 1.77E−02 |
| ENSG00000111640 | GAPDH | 2.16E+00 | 9.25E+00 | 2.84E+00 | 1.30E−02 |
| ENSG00000083845 | RPS5 | 2.12E+00 | 7.39E+00 | 2.45E+00 | 2.26E−02 |
| ENSG00000213160 | KLHL23 | 2.12E+00 | 4.02E+00 | 2.64E+00 | 1.73E−02 |
| ENSG00000143494 | VASH2 | 2.11E+00 | 3.39E+00 | 1.97E+00 | 4.56E−02 |
| ENSG00000123600 | METTL8 | 2.09E+00 | 3.82E+00 | 2.11E+00 | 3.71E−02 |
| ENSG00000173141 | MRPL57 | 2.09E+00 | 4.21E+00 | 2.75E+00 | 1.48E−02 |
| ENSG00000103489 | XYLT1 | 2.09E+00 | 5.30E+00 | 2.56E+00 | 1.93E−02 |
| ENSG00000166086 | JAM3 | 2.08E+00 | 5.62E+00 | 3.47E+00 | 5.44E−03 |
| ENSG00000138758 | SEPT11 | 2.07E+00 | 4.97E+00 | 2.92E+00 | 1.16E−02 |
| ENSG00000233410 | <NA> | 2.06E+00 | 3.81E+00 | 2.17E+00 | 3.39E−02 |
| ENSG00000184117 | NIPSNAP1 | 2.06E+00 | 3.63E+00 | 2.33E+00 | 2.70E−02 |
| ENSG00000084207 | GSTP1 | 2.06E+00 | 4.29E+00 | 2.11E+00 | 3.74E−02 |
| ENSG00000205133 | TRIQK | 2.06E+00 | 3.16E+00 | 2.10E+00 | 3.77E−02 |
| ENSG00000148400 | NOTCH1 | 2.06E+00 | 6.89E+00 | 4.04E+00 | 2.62E−03 |
| ENSG00000111885 | MAN1A1 | 2.06E+00 | 5.23E+00 | 2.55E+00 | 1.97E−02 |
| ENSG00000093217 | XYLB | 2.05E+00 | 3.59E+00 | 2.24E+00 | 3.09E−02 |
| ENSG00000164253 | WDR41 | 2.05E+00 | 4.53E+00 | 2.79E+00 | 1.39E−02 |
| ENSG00000128791 | TWSG1 | 2.05E+00 | 3.60E+00 | 2.39E+00 | 2.47E−02 |
| ENSG00000059573 | ALDH18A1 | 2.04E+00 | 4.55E+00 | 2.67E+00 | 1.65E−02 |
| ENSG00000115268 | RPS15 | 2.04E+00 | 7.19E+00 | 2.23E+00 | 3.13E−02 |
| ENSG00000231500 | RPS18 | 2.03E+00 | 6.63E+00 | 2.33E+00 | 2.69E−02 |
| ENSG00000140563 | MCTP2 | 2.03E+00 | 4.35E+00 | 2.46E+00 | 2.24E−02 |
| ENSG00000168961 | LGALS9 | 2.03E+00 | 3.70E+00 | 2.35E+00 | 2.62E−02 |
| ENSG00000159199 | ATP5MC1 | 2.03E+00 | 2.88E+00 | 1.93E+00 | 4.89E−02 |
| ENSG00000186918 | ZNF395 | 2.03E+00 | 3.12E+00 | 1.97E+00 | 4.59E−02 |
| ENSG00000133935 | ERG28 | 2.02E+00 | 3.58E+00 | 2.05E+00 | 4.05E−02 |
| ENSG00000114942 | EEF1B2 | 2.02E+00 | 7.00E+00 | 2.80E+00 | 1.37E−02 |
| ENSG00000151914 | DST | 2.02E+00 | 5.62E+00 | 2.41E+00 | 2.39E−02 |
| ENSG00000183527 | PSMG1 | 2.01E+00 | 4.52E+00 | 1.97E+00 | 4.55E−02 |
| ENSG00000228474 | OST4 | 2.00E+00 | 4.80E+00 | 2.74E+00 | 1.50E−02 |
| ENSG00000188636 | RTL6 | 1.99E+00 | 5.23E+00 | 2.99E+00 | 1.05E−02 |
| ENSG00000119979 | FAM45A | 1.98E+00 | 4.55E+00 | 2.15E+00 | 3.48E−02 |
| ENSG00000071082 | RPL31 | 1.98E+00 | 8.20E+00 | 3.26E+00 | 7.22E−03 |
| ENSG00000281103 | TRG-AS1 | 1.98E+00 | 4.54E+00 | 2.63E+00 | 1.74E−02 |
| ENSG00000059588 | TARBP1 | 1.98E+00 | 6.37E+00 | 2.64E+00 | 1.72E−02 |
| ENSG00000100823 | APEX1 | 1.97E+00 | 7.13E+00 | 3.27E+00 | 7.11E−03 |
| ENSG00000100129 | EIF3L | 1.97E+00 | 6.23E+00 | 3.08E+00 | 9.31E−03 |
| ENSG00000172687 | ZNF738 | 1.95E+00 | 3.90E+00 | 2.32E+00 | 2.75E−02 |
| ENSG00000144711 | IQSEC1 | 1.95E+00 | 4.79E+00 | 2.64E+00 | 1.71E−02 |
| ENSG00000108298 | RPL19 | 1.92E+00 | 7.69E+00 | 3.05E+00 | 9.72E−03 |
| ENSG00000169855 | ROBO1 | 1.91E+00 | 4.39E+00 | 2.40E+00 | 2.43E−02 |
| ENSG00000144354 | CDCA7 | 1.90E+00 | 6.46E+00 | 2.89E+00 | 1.21E−02 |
| ENSG00000142657 | PGD | 1.90E+00 | 5.08E+00 | 2.59E+00 | 1.86E−02 |
| ENSG00000152556 | PFKM | 1.89E+00 | 3.46E+00 | 1.96E+00 | 4.66E−02 |
| ENSG00000157657 | ZNF618 | 1.89E+00 | 4.46E+00 | 2.45E+00 | 2.26E−02 |
| ENSG00000174989 | FBXW8 | 1.88E+00 | 3.76E+00 | 1.96E+00 | 4.62E−02 |
| ENSG00000135272 | MDFIC | 1.87E+00 | 5.31E+00 | 2.32E+00 | 2.72E−02 |
| ENSG00000105373 | NOP53 | 1.87E+00 | 5.42E+00 | 2.80E+00 | 1.37E−02 |
| ENSG00000149639 | SOGA1 | 1.87E+00 | 5.24E+00 | 2.75E+00 | 1.46E−02 |
| ENSG00000135446 | CDK4 | 1.85E+00 | 3.66E+00 | 1.94E+00 | 4.73E−02 |
| ENSG00000142937 | RPS8 | 1.84E+00 | 7.87E+00 | 1.99E+00 | 4.44E−02 |
| ENSG00000114738 | MAPKAPK3 | 1.83E+00 | 5.09E+00 | 2.55E+00 | 1.96E−02 |
| ENSG00000003400 | CASP10 | 1.83E+00 | 5.19E+00 | 2.34E+00 | 2.64E−02 |
| ENSG00000163597 | SNHG16 | 1.82E+00 | 3.88E+00 | 2.04E+00 | 4.12E−02 |
| ENSG00000145741 | BTF3 | 1.82E+00 | 5.95E+00 | 2.76E+00 | 1.46E−02 |
| ENSG00000184635 | ZNF93 | 1.82E+00 | 3.73E+00 | 1.96E+00 | 4.64E−02 |
| ENSG00000148840 | PPRC1 | 1.82E+00 | 5.44E+00 | 2.08E+00 | 3.87E−02 |
| ENSG00000115850 | LCT | 1.81E+00 | 4.20E+00 | 2.12E+00 | 3.65E−02 |
| ENSG00000117174 | ZNHIT6 | 1.81E+00 | 5.43E+00 | 2.66E+00 | 1.68E−02 |
| ENSG00000118276 | B4GALT6 | 1.80E+00 | 4.99E+00 | 2.27E+00 | 2.95E−02 |
| ENSG00000186468 | RPS23 | 1.80E+00 | 4.93E+00 | 1.93E+00 | 4.81E−02 |
| ENSG00000167085 | PHB | 1.79E+00 | 4.91E+00 | 2.01E+00 | 4.32E−02 |
| ENSG00000147403 | RPL10 | 1.78E+00 | 8.07E+00 | 2.39E+00 | 2.45E−02 |
| ENSG00000144736 | SHQ1 | 1.78E+00 | 5.46E+00 | 2.16E+00 | 3.45E−02 |
| ENSG00000198561 | CTNND1 | 1.78E+00 | 5.95E+00 | 2.04E+00 | 4.12E−02 |
| ENSG00000118495 | PLAGL1 | 1.77E+00 | 4.75E+00 | 2.20E+00 | 3.24E−02 |
| ENSG00000181163 | NPM1 | 1.77E+00 | 9.24E+00 | 2.06E+00 | 3.96E−02 |
| ENSG00000198755 | RPL10A | 1.75E+00 | 7.60E+00 | 2.03E+00 | 4.19E−02 |
| ENSG00000136636 | KCTD3 | 1.75E+00 | 5.00E+00 | 2.28E+00 | 2.90E−02 |
| ENSG00000149554 | CHEK1 | 1.74E+00 | 5.33E+00 | 1.95E+00 | 4.69E−02 |
| ENSG00000148303 | RPL7A | 1.74E+00 | 7.94E+00 | 1.91E+00 | 4.94E−02 |
| ENSG00000118181 | RPS25 | 1.73E+00 | 7.59E+00 | 2.57E+00 | 1.89E−02 |
| ENSG00000102221 | JADE3 | 1.73E+00 | 4.55E+00 | 1.95E+00 | 4.71E−02 |
| ENSG00000143641 | GALNT2 | 1.73E+00 | 6.43E+00 | 2.57E+00 | 1.91E−02 |
| ENSG00000141076 | UTP4 | 1.72E+00 | 4.90E+00 | 2.10E+00 | 3.78E−02 |
| ENSG00000104738 | MCM4 | 1.72E+00 | 6.16E+00 | 2.07E+00 | 3.91E−02 |
| ENSG00000174444 | RPL4 | 1.71E+00 | 9.07E+00 | 2.33E+00 | 2.68E−02 |
| ENSG00000172716 | SLFN11 | 1.69E+00 | 5.87E+00 | 2.39E+00 | 2.47E−02 |
| ENSG00000102898 | NUTF2 | 1.69E+00 | 4.97E+00 | 1.92E+00 | 4.88E−02 |
| ENSG00000134986 | NREP | 1.64E+00 | 8.64E+00 | 2.75E+00 | 1.47E−02 |
| ENSG00000121210 | TMEM131L | 1.63E+00 | 8.51E+00 | 2.54E+00 | 2.00E−02 |
| ENSG00000204472 | AIF1 | 1.63E+00 | 5.51E+00 | 1.95E+00 | 4.71E−02 |
| ENSG00000197961 | ZNF121 | 1.61E+00 | 5.53E+00 | 2.01E+00 | 4.28E−02 |
| ENSG00000115365 | LANCL1 | 1.60E+00 | 5.70E+00 | 2.11E+00 | 3.73E−02 |
| ENSG00000139083 | ETV6 | 1.60E+00 | 7.27E+00 | 2.54E+00 | 1.98E−02 |
| ENSG00000197635 | DPP4 | 1.57E+00 | 6.22E+00 | 2.05E+00 | 4.06E−02 |
| ENSG00000103222 | ABCC1 | 1.54E+00 | 7.15E+00 | 1.93E+00 | 4.84E−02 |
| ENSG00000153283 | CD96 | 1.51E+00 | 7.74E+00 | 1.94E+00 | 4.76E−02 |
| ENSG00000110700 | RPS13 | 1.50E+00 | 7.69E+00 | 2.06E+00 | 3.99E−02 |
| ENSG00000113648 | H2AFY | 1.48E+00 | 8.26E+00 | 2.18E+00 | 3.33E−02 |

### REFERENCES

Schatz, D. G., M. A. Oettinger, and D. Baltimore. 1989. The V(D)J recombination activating gene, RAG-1. Cell 59: 1035-1048.
Oettinger, M. A., D. G. Schatz, C. Gorka, and D. Baltimore. 1990. RAG-1 and RAG-2, adjacent genes that synergistically activate V(D)J recombination. Science 248: 1517-1523.
Jones, J. M., and M. Gellert. 2004. The taming of a transposon: V(D)J recombination and the immune system. Immunol Rev 200: 233-248.
Smith, A. L., J. N. F. Scott, and J. Boyes. 2019. The ESC: The Dangerous By-Product of V(D)J Recombination. Front Immunol 10: 1572.
Bories, J. C., J. M. Cayuela, P. Loiseau, and F. Sigaux. 1991. Expression of human recombination activating genes (RAG1 and RAG2) in neoplastic lymphoid cells: correlation with cell differentiation and antigen receptor expression. Blood 78: 2053-2061.
Li, Y., P. M. Brauer, J. Singh, S. Xhiku, K. Yoganathan, J. C. Zuniga-Pflucker, and M. K. Anderson. 2017. Targeted Disruption of TCF12 Reveals HEB as Essential in Human Mesodermal Specification and Hematopoiesis. Stem Cell Reports 9: 779-795.
Kennedy, M., G. Awong, C. M. Sturgeon, A. Ditadi, R. LaMotte-Mohs, J. C. Zuniga-Pflucker, and G. Keller. 2012. T lymphocyte potential marks the emergence of definitive hematopoietic progenitors in human pluripotent stem cell differentiation cultures. Cell Rep 2: 1722-1735.
Villey, I., P. Quartier, F. Selz, and J. P. de Villartay. 1997. Germ-line transcription and methylation status of the TCR-J alpha locus in its accessible configuration. Eur J Immunol 27: 1619-1625.
Casero, D., S. Sandoval, C. S. Seet, J. Scholes, Y. Zhu, V. L. Ha, A. Luong, C. Parekh, and G. M. Crooks. 2015. Long non-coding RNA profiling of human lymphoid progenitor cells reveals transcriptional divergence of B cell and T cell lineages. Nat Immunol 16: 1282-1291.
He, Y. W. 2000. The role of orphan nuclear receptor in thymocyte differentiation and lymphoid organ development. Immunol Res 22: 71-82.
Lefebvre, J. M., M. C. Haks, M. O. Carleton, M. Rhodes, G. Sinnathamby, M. C. Simon, L. C. Eisenlohr, L. A. Garrett-Sinha, and D. L. Wiest. 2005. Enforced expression of Spi-B reverses T lineage commitment and blocks beta-selection. J Immunol 174: 6184-6194.
Klein, F., M. Mitrovic, J. Roux, C. Engdahl, L. von Muenchow, L. Alberti-Servera, H. J. Fehling, P. Pelczar, A. Rolink, and P. Tsapogas. 2019. The transcription factor Duxbl mediates elimination of pre-T cells that fail beta-selection. J Exp Med 216: 638-655.
Sicinska, E., I. Aifantis, L. Le Cam, W. Swat, C. Borowski, Q. Yu, A. A. Ferrando, S. D. Levin, Y. Geng, H. von Boehmer, and P. Sicinski. 2003. Requirement for cyclin D3 in lymphocyte development and T cell leukemias. Cancer Cell 4: 451-461.
von Boehmer, H., I. Aifantis, J. Feinberg, O. Lechner, C. Saint-Ruf, U. Walter, J. Buer, and O. Azogui. 1999. Pleiotropic changes controlled by the pre-T-cell receptor. Curr Opin Immunol 11: 135-142.
Michie, A. M., and J. C. Zuniga-Pflucker. 2002. Regulation of thymocyte differentiation: pre-TCR signals and beta-selection. Semin Immunol 14: 311-323.
Rothenberg, E. V., and T. Taghon. 2005. Molecular genetics of T cell development. Annu Rev Immunol 23: 601-649.
Carrasco, Y. R., M. N. Navarro, V. G. de Yebenes, A. R. Ramiro, and M. L. Toribio. 2002. Regulation of surface expression of the human pre-T cell receptor complex. Semin Immunol 14: 325-334.
Blom, B., M. C. Verschuren, M. H. Heemskerk, A. Q. Bakker, E. J. van Gastel-Mol, I. L. Wolvers-Tettero, J. J. van Dongen, and H. Spits. 1999. TCR gene rearrangements and expression of the pre-T cell receptor complex during human T-cell differentiation. Blood 93: 3033-3043.
Carrasco, Y. R., C. Trigueros, A. R. Ramiro, V. G. de Yebenes, and M. L. Toribio. 1999. Beta-selection is associated with the onset of CD8beta chain expression on CD4(+)CD8alphaalpha(+) pre-T cells during human intrathymic development. Blood 94: 3491-3498.
Miyazaki, M., K. Miyazaki, M. Itoi, Y. Katoh, Y. Guo, R. Kanno, Y. Katoh-Fukui, H. Honda, T. Amagai, M. van Lohuizen, H. Kawamoto, and M. Kanno. 2008. Thymocyte proliferation induced by pre-T cell receptor signaling is maintained through polycomb gene product Bmi-1-mediated Cdkn2a repression. Immunity 28: 231-245.
Roszkowski, J. J., D. C. Yu, M. P. Rubinstein, M. D. McKee, D. J. Cole, and M. I. Nishimura. 2003. CD8-independent tumor cell recognition is a property of the T cell receptor and not the T cell. J Immunol 170: 2582-2589.
Benveniste, P. M., S. Roy, M. Nakatsugawa, E. L. Y. Chen, L. Nguyen, D. G. Millar, P. S. Ohashi, N. Hirano, E. J. Adams, and J. C. Zuniga-Pflucker. 2018. Generation and molecular recognition of melanoma-associated antigen-specific human gammadelta T cells. Sci Immunol 3.

1. A method of generating stem or progenitor cells unable to undergo T cell receptor (TCR) gene rearrangements (TCR), the method comprising:
(a) culturing a sample comprising stem cells or progenitor cells,
wherein expression of at least one gene or protein required for V(D)J recombination in the stem cells or progenitor cells is reduced or eliminated compared to wildtype stem cells or progenitor cells.

2. The method of claim 1, wherein the method further comprises (b) isolating cells of the T cell lineage.

3. The method of claim 1, wherein the at least one gene or protein required for V(D)J recombination is RAG1 and/or RAG2.

4. The method of claim 1, wherein the at least one gene or protein required for V(D)J recombination is selected from the group consisting of Artemis, DNA-dependent protein kinase (DNA-PK), X-ray repair cross-complementing protein 4 (XRCC4), DNA ligase IV, non-homologous end-joining factor 1 (NHEJ1), Paralog of XRCC4 and XLF (PAXX), DNA polymerase λ and DNA polymerase μ.

5. The method of claim 1, wherein the stem cells are pluripotent stem cells, optionally embryonic stem cells or induced pluripotent stem cells (iPSCs).

6. (canceled)

7. The method of claim 1, wherein the stem cells or progenitor cells are human cells.

8. The method of claim 1, wherein the cells of the T cell lineage are progenitor T (proT) cells, CD4+CD8+ double positive cells, CD4+CD8+CD3+ double positive cells, CD8+CD3+ single positive cells or CD4+CD3+ single positive cells.

9. (canceled)

10. (canceled)

11. The method of claim 1, further comprising engineering the stem cells or progenitor cells or the cells of the T cell lineage to comprise at least one of a nucleic acid encoding a T cell receptor (TCR), a nucleic acid encoding a TCRβ chain and a nucleic acid encoding a chimeric antigen receptor (CAR), optionally wherein the stem cells or progenitor cells or the cells of the T cell lineage express the at least one of the T cell receptor (TCR), the TCRβ chain and the chimeric antigen receptor (CAR).

12.-14. (canceled)

15. The method of claim 11, wherein the TCR or CAR confers specificity to an antigen, optionally a tumor-associated antigen, viral antigen or self antigen.

16. A cell of the T cell lineage, wherein the cell is generated by the method of claim 1.

17. (canceled)

18. (canceled)

19. A stem or progenitor cell, wherein expression of at least one gene or protein required for V(D)J recombination in the stem cell or progenitor cell is reduced or eliminated compared to a wild-type stem cell or progenitor cell.

20. The stem or progenitor cell of claim 19, wherein the at least one gene or protein required for V(D)J recombination is RAG1 and/or RAG2.

21. The stem or progenitor cell of claim 19, wherein the at least one gene or protein required for V(D)J recombination is selected from the group consisting of Artemis, DNA-dependent protein kinase (DNA-PK), X-ray repair cross-complementing protein 4 (XRCC4), DNA ligase IV, non-homologous end-joining factor 1 (NHEJ1), Paralog of XRCC4 and XLF (PAXX), DNA polymerase λ and DNA polymerase μ.

22. The stem or progenitor cell of claim 1, further comprising at least one of a nucleic acid encoding a T cell receptor (TCR), a nucleic acid encoding a TCRβ chain and a nucleic acid encoding a chimeric antigen receptor (CAR).

23. (canceled)

24. The stem or progenitor cell of claim 1, wherein the stem cell is a pluripotent stem cell, optionally an embryonic stem cell or induced pluripotent stem cell (iPSC).

25.-27. (canceled)

28. A kit comprising (i) a stem or progenitor cell of claim 19 and (ii) instructions for use of the stem or progenitor cell of claim 19 for generating cells of the T cell lineage.

29. A method of treating a disease or condition in a subject comprising:
(i) culturing a sample comprising stem cells or progenitor cells, wherein expression of at least one gene or protein required for V(D)J recombination in the stem cells or progenitor cells is reduced or eliminated compared to wildtype stem cells or progenitor cells, or
culturing a sample comprising stem cells or progenitor cells, and isolating cells of the T cell lineage wherein expression of at least one gene or protein required for V(D)J recombination in the stem cells or progenitor cells is reduced or eliminated compared to wildtype stem cells or progenitor cells, and
and
(ii) administering an effective amount of the cells or progenitor cells or the cells of the T cell lineage to a subject in need thereof,
wherein the stem cells or progenitor cells or the cells of the T cell lineage are engineered to comprise at least one of a nucleic acid encoding a T cell receptor (TCR) and a chimeric antigen receptor (CAR) that confers specificity to an antigen.

30. (canceled)

31. The method of claim 29, wherein the at least one gene or protein required for V(D)J recombination is RAG1 and/or RAG2.

32. The method of claim 29, wherein the at least one gene or protein required for V(D)J recombination is selected from the group consisting of Artemis, DNA-dependent protein kinase (DNA-PK), X-ray repair cross-complementing protein 4 (XRCC4), DNA ligase IV, non-homologous end-joining factor 1 (NHEJ1), Paralog of XRCC4 and XLF (PAXX), DNA polymerase λ and DNA polymerase p.

33. The method of claim 29, wherein the disease is cancer and the antigen is a tumor-associated antigen.

34.-55. (canceled)
